(19) United States
(12) Patent Application Publication
(10) Pub. No.: US 2023/0285287 A1
(43) Pub. Date: Sep. 14, 2023

(54) PHARMACEUTICAL COMPOSITIONS

(71) Applicant: DOUGLAS PHARMACEUTICALS LIMITED, Auckland (NZ)

(72) Inventors: Fergus BINNIE, Auckland (NZ); Peter SURMAN, Auckland (NZ)

(21) Appl. No.: 17/776,255

(22) PCT Filed: Nov. 26, 2020
(86) PCT No.: PCT/IB2020/061183

§ 371 Date: May 12, 2022

(30) Foreign Application Priority Data
Nov. 27, 2019 (GB) 1917252.7

(51) Int. Cl. A61K 9/107; A61K 31/513; A61K 47/14; A61K 31/426; A61K 47/10; A61K 9/48; A61K 47/26; A61K 47/44

(52) U.S. Cl. CPC A61K 9/107; A61K 31/513; A61K 47/14; A61K 31/426; A61K 47/10; A61K 9/4825; A61K 47/26; A61K 47/44

(57) ABSTRACT

The present invention relates to self-emulsifying pharmaceutical compositions comprising an unsaturated free fatty acid, at least two emulsifiers and at least one active pharmaceutical ingredient; to the use of said pharmaceutical compositions as a medicament; and to processes for the preparation of said compositions.

The present invention relates to self-emulsifying pharmaceutical compositions comprising an unsaturated free fatty acid, at least two emulsifiers and at least one active pharmaceutical ingredient (lopinavir); methods for their manufacture; and the use of said pharmaceutical compositions as a medicament. In particular, pharmaceutical compositions are provided comprising lopinavir and ritonavir for use in treating and/or inhibiting the progression of diseases and/or disorders such as HPV related dysplasia of the cervix.

### BACKGROUND OF THE INVENTION

Self-emulsifying drug delivery systems (SEDDS) form emulsions as a result of the chemical properties of the components of the delivery system, rather than as a result of mechanical mixing. Typically, a lipophilic drug is dissolved in an oil-based formulation also containing surfactants. The formulation may be filled into soft or hard capsules for oral administration. When the SEDDS formulation contacts aqueous environments, e.g. in the form of gastrointestinal fluids after oral administration, it spontaneously forms oil-in-water emulsions, which facilitate drug absorption.

Marketed SEDDS formulations include those comprising the drug cyclosporine formulated for oral administration and sold under the product names of Neoral® and Gengraf®. Norvir® and Fortovase® have been marketed as SEDDS formulations containing the HIV protease inhibitors ritonavir and saquinavir respectively [Gibaud & Attivi, Expert Opinion on Drug Delivery 2012, 937-951].

A self-emulsifying composition is an isotropic mixture typically containing an oil, a surfactant, and optionally a cosurfactant and/or a cosolvent. Examples of oils used in SEDDS include long chain triglycerides, such as sesame oil, soybean oil and castor oil, medium chain triglycerides, silicon oil, fatty acids and fatty alcohols. The oil is required to dissolve a lipophilic, or poorly water-soluble, drug. Surfactants or emulsifiers are added to ensure efficient self-dispersibility and stability of the formed oil-in-water emulsion. Non-ionic surfactants can be classified by their hydrophilic-lipophilic balance (HLB) on a scale of 1 to 20 with 1 being lipophilic and 20 being hydrophilic [Griffin, J. Soc. Cosmetic Chem. 1954, 249-256]. Examples of surfactants include ethers of polyols and vegetable oils (such as polyoxyl 35 castor oil (Cremophor® EL) and polyoxyl 40 hydrogenated castor oil (Cremophor® RH 40)), polyol glycerides (such as lauroyl macrogol-32 glycerides (Gelucire® 44/14) and polyol esters (such as polysorbates (e.g. Tween® 20 or Tween® 80) or polyethylene glycol stearates), as well as more lipophilic surfactants including propylene glycol monoesters (such as propylene glycol monolaurate or propylene glycol monocaprylate), glycol monoethers (such as Transcutol® or Carbitol®) and mono- and diglycerides. The surfactant content of SEDDS is generally in the range 30% to 60% by weight to provide stable emulsions [Kovvasu et al., Asian J. Pharm. 2019, 73-84]. Cosolvents which may be employed include water, ethanol, glycerine and polyethylene glycol [Gibaud & Attivi, Expert Opinion on Drug Delivery 2012, 937-951].

The present invention is based on work carried out by the inventors to formulate a self-emulsifying composition comprising at least one active pharmaceutical ingredient (lopinavir). They have unexpectedly found that when at least two emulsifiers with particular properties are combined with an unsaturated free fatty acid and at least one active pharmaceutical ingredient (lopinavir), then a SEDDS formulation is obtained having unexpectedly good properties (e.g. drug dissolution and therefore potentially superior bioavailability properties) even though the formulations contain relatively low total emulsifier contents.

The use of an unsaturated free fatty acid in the composition is advantageous as the quality of the free fatty acid in the composition can be controlled, such as identity, amount and purity of the free fatty acid. In contrast, other excipients, such as vegetable oils and polysorbates, may contain low and variable levels of free fatty acids. The free fatty acid composition of vegetable oil and polysorbate, such as the identity of the free fatty acid and the amount, can vary from batch to batch, and over time. Advantageously, in one embodiment, the active pharmaceutical ingredient is soluble in the unsaturated free fatty acid and no heat above room temperature is required to achieve solubilisation. This is particularly advantageous when using an active pharmaceutical ingredient (such as lopinavir) that is prone to degradation; especially when the extent and/or rate of degradation, such as degradation by oxidation and/or hydrolysis is increased when the active pharmaceutical ingredient is exposed to heat.

Conveniently, the compositions of the invention are useful in the treatment of cancer. Many different forms of cancer exist, and it is believed that there are many different causes of the disease. The incidence of cancer varies, but it represents the second highest cause of mortality, after heart disease, in most developed countries.

Human tumour viruses are recognised to be a major cause of human cancer, and there is a great deal of evidence which supports the contention that these viruses cause cancer by inducing genetic instability in infected cells. Indeed, both the human T-cell leukemia virus type 1 (HTLV1) Tax and the human papilloma virus type 16 (HPV16) E6 oncoproteins are known to induce genetic instability producing abnormal numbers of centrosomes, multinucleation and nuclear atypia.

Invasive cervical cancer (ICC) is an example of a cancer associated with viral infection which causes >270,000 deaths per annum with over 85% of these occurring in low resource countries. Infection with high-risk types of HPV has been established as the main aetiological agent for ICC. The development of ICC can take 10-20 years and is preceded by HPV related pre-invasive pathology which is characterised as either low-grade (CIN1) or high-grade cervical intraepithelial neoplasia (CIN2/3). Lesions can be screened for by cervical cytology testing where they are diagnosed (or graded) as either borderline atypical squamous cells of undetermined significance (ASCUS), low-grade squamous intraepithelial lesions (LSIL) or high-grade squamous intraepithelial lesions (HSIL).

The reduction in ICC related mortality in the developed world has been largely dependent on organised cytology screening and similar trends in cervical cancer mortality have been achieved by organised single screen and treatment in the rest of the world. However, in the poorer nations lack of resources and health education means that most pre-invasive cervical disease remains undiagnosed and untreated. Thus, where resources are limited, low-cost screening and treatment options are clearly a high priority.

Current treatment options in clinical practice are either by ablative (destructive) or excisional modalities. Systematic reviews have demonstrated that these treatment modalities have similar success rates but have different morbidities. In the developed world, Large Loop Excision of the Transformation Zone LLETZ (aka loop electrosurgical excision procedure—LEEP) is used in the majority of colposcopy clinics. Over 80% of these procedures are performed under local analgesia and the whole of the transformation zone is available for subsequent histological examination. The procedure is associated with a risk of primary/secondary haemorrhage, prolonged discharge, infection and a risk of preterm delivery in subsequent pregnancies. The former side effects can be problematic particularly in low resource countries. Ablative treatment in the form of cold coagulation and cryotherapy are often advocated for use in low resource countries since these are low cost, require minimal infrastructure and can be carried out by trained non-medical health professionals. However, some studies have suggested that cryotherapy has a higher failure rate compared to other treatment modalities.

There are a variety of locally-applied, non-surgical approaches which have been evaluated for the treatment of cervical dysplasia including; photodynamic therapy (PDT), off-licence use of the anti-cytomegalovirus (CMV) drug cidofovir; local application of the immune activator Imiquimod and direct application of the cytotoxic drug 5 flurouracil (5FU). Although some of these alternative treatment modalities show promise, their treatment outcomes are inferior to the reported 80-95% success rates obtained in quality assured colposcopy units.

An effective, inexpensive, non-surgical, self-administered treatment for HPV related cervical dysplasia would have great potential particularly in low resource settings. Furthermore, improved compliance with self-administered treatment would be enhanced, if the side effects are minimised.

A recent advance in the treatment of cancers caused by viruses is disclosed in WO2015/059485 which describes the protease inhibitors, lopinavir and ritonavir (which had previously been used as orally ingested medicaments for the clinical management of retroviral infections such as HIV) as being clinically useful for topical administration to tissues to prevent or treat malignancies caused by human papilloma virus. The authors reported that soft capsules of KALETRA® (which is marketed by Abbott/Abbvie for the treatment of HIV infections by oral administration) can be administered topically (e.g. inserted into the vagina for treatment of the cervix) for the prevention or treatment of cancerous conditions, for the prevention or treatment of oncogenic viral infections and for the prevention or treatment of benign proliferative orders. KALETRA® is available for oral consumption as a solution comprising 80 mg lopinavir and 20 mg ritonavir per millilitre or as a soft capsule for oral administration that comprises 133.3 mg lopinavir and 33.3 mg ritonavir (4:1 wt/wt ratio of lopinavir:ritonavir). The solution additionally contains alcohol (42% w/w), high fructose corn syrup, propylene glycol, purified water, glycerol, povidone, flavourings, polyoxyl 40 hydrogenated castor oil, acesulfame potassium, saccharin sodium, sodium chloride, peppermint oil, sodium citrate, citric acid, and menthol. The soft capsule contents contain, along with lopinavir and ritonavir, oleic acid, propylene glycol, polyoxyl 35 castor oil (Cremophor® EL), and purified water (KALETRA® Summary of Product Characteristics, EMA; WO2002/096395).

The compositions of the invention offer significant benefits when compared to previous formulations. Accordingly, in one particular embodiment, when the composition comprising lopinavir and ritonavir is administered topically to a mucosal surface (e.g. inserted into the vagina for treatment of the cervix) or orally, the composition can be used to treat and prevent cancerous conditions, for the prevention or treatment of oncogenic viral infections, and for the prevention or treatment of benign proliferative orders.

### SUMMARY OF INVENTION

Disclosed herein are self-emulsifying pharmaceutical compositions comprising an unsaturated free fatty acid, at least two emulsifiers and at least one active pharmaceutical ingredient (API) (lopinavir and ritonavir).

According to a first aspect of the invention, there is provided a self-emulsifying pharmaceutical composition comprising:
a. an unsaturated free fatty acid;
b. at least two emulsifiers; and
c. at least one active pharmaceutical ingredient (lopinavir);
   wherein the at least two emulsifiers comprise at least a first emulsifier which has a HLB value greater than about 14 and at least a second emulsifier which has a HLB value less than about 6; and wherein the total emulsifier content is less than 30% by weight of the total composition.

According to a second aspect, there is provided a process to manufacture a self-emulsifying pharmaceutical composition of the first aspect, the process comprising:
a) incorporating at least one active pharmaceutical ingredient (lopinavir) in an unsaturated free fatty acid; and
b) incorporating at least two emulsifiers into the mixture obtained from step a) to provide a self-emulsifying composition;
   wherein the at least two emulsifiers comprise at least a first emulsifier which has a HLB value greater than about 14 and at least a second emulsifier which has a HLB value less than about 6; and wherein the total emulsifier content is less than 30% by weight of the total composition.

According to a third aspect of the invention there is provided a pharmaceutical composition according to the first aspect of the invention for use as a medicament. In one embodiment, the pharmaceutical composition is used as a medicament for treating and/or inhibiting the development or progression of diseases and/or disorders. In one embodiment, the pharmaceutical composition is used as a medicament for treating and/or inhibiting the development or progression of cancers and/or benign proliferative disorders. In one embodiment, the pharmaceutical composition comprises an effective amount of the at least one active pharmaceutical ingredient (lopinavir). In one embodiment, the pharmaceutical composition comprises an effective amount of the at least one active pharmaceutical ingredient (lopinavir) for treating and/or inhibiting the development or progression of a disease or disorder. In one embodiment, the pharmaceutical composition comprises an effective amount of the at least one active pharmaceutical ingredient (lopinavir) for treating and/or inhibiting the development or progression of cancers and/or benign proliferative disorders. In a further embodiment, the pharmaceutical composition comprises an effective amount of the at least one active pharmaceutical ingredient (lopinavir and/or ritonavir) for treating a Human Papilloma Virus (HPV) infection with or without attendant abnormal pathology. In one embodiment, the pharmaceutical composition is used as a medicament for treating and/or inhibiting the development of early stage neoplasias. In one embodiment, the pharmaceutical composition is used as a medicament for treating or preventing the development of HPV related cervical, vulval, vaginal, penile, anal, oral or laryngeal neoplasias and/or warts. In one embodiment, the pharmaceutical composition is for use as a medicament for treating or preventing the development of cervical neoplasias.

According to a fourth aspect of the invention there is provided a method of treating and/or inhibiting the development or progression of diseases and/or disorders in a subject in need of such treatment or inhibition comprising administering a therapeutically effective amount of a composition according to the first aspect of the invention. In one embodiment, there is provided a method of treating and/or inhibiting the development or progression of cancers and/or benign proliferative disorders in a subject in need of such treatment or inhibition comprising administering a therapeutically effective amount of a composition according to the first aspect of the invention. In one embodiment, the cancer or disorder is caused or induced by a human papilloma virus (HPV). In a further embodiment, there is a provided a method of treating a Human Papilloma Virus (HPV) infection with or without attendant abnormal pathology in a subject in need of such treatment or inhibition comprising administering a therapeutically effective amount of the pharmaceutical composition according to the first aspect of the invention. In one embodiment, there is provided a method of treating and/or inhibiting the development of early stage neoplasias in a subject in need of such treatment or inhibition comprising administering a therapeutically effective amount of the pharmaceutical composition according to the first aspect of the invention. In one embodiment, there is provided a method of treating or preventing the development of HPV related cervical, vulval, vaginal, penile, anal, oral or laryngeal neoplasias and/or warts in a subject in need of such treatment or inhibition comprising administering a therapeutically effective amount of the pharmaceutical composition according to the first aspect of the invention. In one embodiment, there is provided a method of treating or preventing the development of cervical neoplasias in a subject in need of such treatment or inhibition comprising administering a therapeutically effective amount of the pharmaceutical composition according to the first aspect of the invention.

Conveniently, the cancer or benign proliferative disorder is caused by a viral infection, more preferably by an oncogenic virus and in particular human tumour viruses such as HPV.

Conveniently, the invention concerns treating a subject having an HPV related dysplasia of the cervix comprising administering to said subject a therapeutically effective dose of the disclosed pharmaceutical compositions.

### DETAILED DESCRIPTION

The disclosed compositions, processes of manufacture and methods may be understood more readily by reference to the following detailed description which form a part of this disclosure. It is to be understood that the disclosed compositions, processes of manufacture and methods are not limited to the specific compositions, processes of manufacture and methods described and/or shown herein, and that the terminology used herein is for the purpose of describing particular embodiments by way of example only and is not intended to be limiting of the claimed compositions, processes of manufacture and methods.

Reference to a particular numerical value includes at least that particular value, unless the context clearly dictates otherwise. When a range of values is expressed, another embodiment includes from the one particular value and/or to the other particular value. Further, reference to values stated in ranges include each and every value within that range. All ranges are inclusive and combinable.

It is to be appreciated that certain features of the disclosed compositions, processes of manufacture and methods which are, for clarity, described herein in the context of separate embodiments, may also be provided in combination in a single embodiment. Conversely, various features of the disclosed compositions, processes of manufacture and methods that are, for brevity, described in the context of a single embodiment, may also be provided separately or in any sub-combination.

As used herein, the singular forms “a,” “an,” and “the” include the plural.

The following abbreviations are used herein: human papilloma virus (HPV); Atypical squamous cells of undetermined significance (ASC-US); Low grade squamous intraepithelial lesion (LSIL); High grade squamous intraepithelial lesion (HSIL); Cervical intraepithelial neoplasia 1 (CIN1); Cervical Intraepithelial neoplasia 2 (CIN2); Cervical intraepithelial neoplasia 3 (CIN3); Carcinoma in situ (CIS); Invasive Cervical Carcinoma (ICC).

When values are expressed as approximations, by use of the antecedent “about,” it will be understood that the particular value forms another embodiment. The term “about” when used in reference to numerical ranges, cut-offs, or specific values is used to indicate that the recited values may vary by up to as much as 10% from the listed value. As many of the numerical values used herein are experimentally determined, it should be understood by those skilled in the art that such determinations can, and often times will, vary among different experiments. The values used herein should not be considered unduly limiting by virtue of this inherent variation. Thus, the term “about” is used to encompass variations of ±10% or less, variations of ±5% or less, variations of ±1% or less, variations of ±0.5% or less, or variations of ±0.1% or less from the specified value.

As used herein, “treating” and like terms refer to reducing the severity and/or frequency of symptoms, eliminating symptoms and/or the underlying cause of said symptoms, reducing the frequency or likelihood of symptoms and/or their underlying cause, delaying, preventing and/or slowing the progression of diseases and/or disorders, such as cancers or benign proliferative disorders, and improving or remediating damage caused, directly or indirectly, by the diseases and/or disorders such as cancers or benign proliferative disorders.

As used herein, the phrase “therapeutically effective dose” refers to an amount of a composition comprising at least one active pharmaceutical ingredient (lopinavir), as described herein, effective to achieve a particular biological or therapeutic result such as, but not limited to, biological or therapeutic results disclosed, described, or exemplified herein. The therapeutically effective dose may vary according to factors such as the disease state, age, sex, and weight of the individual, and the ability of the composition to cause a desired response in a subject. Such results include, but are not limited to, the reduction, remission, and/or regression of the benign or malignant disease or prevention of the development of the benign or malignant disease, as determined by any means suitable in the art.

As used herein, “subject” includes a vertebrate, mammal, domestic animal or preferably a human being.

### Pharmaceutical Compositions

According to a first aspect of the invention, there is provided a self-emulsifying pharmaceutical composition comprising:
a. an unsaturated free fatty acid;
b. at least two emulsifiers; and
c. at least one active pharmaceutical ingredient (lopinavir);
   wherein the at least two emulsifiers comprise at least a first emulsifier which has a HLB value greater than about 14 and at least a second emulsifier which has a HLB value less than about 6; and wherein the total emulsifier content is less than 30% by weight of the total composition.

A self-emulsifying composition, as used herein, refers to a fat- or oil-based composition which, when introduced into water or aqueous environments, emulsifies spontaneously to produce an oil-in-water or water-in-oil emulsion. A self-emulsifying pharmaceutical composition refers to a self-emulsifying composition containing pharmaceutically acceptable excipients.

A free fatty acid, as used herein, refers to fatty acids which are not attached to a glycerol backbone, i.e., the fatty acid is not part of a glyceride. One advantage of the pharmaceutical composition comprising a free fatty acid is that the identity, amount and purity of the free fatty acid used to manufacture the pharmaceutical composition can be controlled. An unsaturated free fatty acid is a free fatty acid wherein there is at least one double bond between carbon atoms in the fatty acid.

It is to be understood that free fatty acids products that are commercially available may contain small amounts of other fatty acids. For example, oleic acid typically contains 7-12% saturated free fatty acids, such as stearic and palmitic acid, together with other unsaturated free fatty acids, such as linoleic acid (Handbook of Pharmaceutical Excipients, 2^{nd} Edition, see entry for Oleic acid). The term unsaturated free fatty acid is to be understood as meaning the unsaturated free fatty acid is of Pharmacopeia grade, such as the US Pharmacopeia and/or the British Pharmacopeia, and that the unsaturated free fatty acid may contain small amounts of other free fatty acids.

In one embodiment, of the total unsaturated fatty acid (bound and free unsaturated fatty acid) present within the composition, at least 90% by weight, such as at least 95% by weight such as at least 98% by weight, such as at least 99% by weight, or such as at least 99.5% by weight, is in the free form, i.e., not esterified or bound to other components such as glycerol.

In one embodiment, the unsaturated free fatty acid is not in the form of a triglyceride or polysorbate.

In one embodiment, the unsaturated free fatty acid has a melting point below about 25° C. In one embodiment, the unsaturated free fatty acid is selected from oleic acid, linoleic acid, alpha-linoleic acid, palmitoleic acid, gondoic acid, and ricinoleic acid. In a preferred embodiment, the unsaturated free fatty acid is oleic acid.

In an embodiment, the unsaturated free fatty acid is present in the pharmaceutical composition at a level of at least 25% by weight of the total pharmaceutical composition, such as at least 35% by weight, at least 45% by weight, at least 50% by weight, at least 55% by weight, at least 60% by weight, at least 65% by weight, or at least 70% by weight of the total pharmaceutical composition.

In an embodiment, the unsaturated free fatty acid is present in the pharmaceutical composition at a level of about 25% to about 85% by weight of the total pharmaceutical composition, such as about 40% to about 85% by weight, about 50% to about 85% by weight, about 55% to 85% by weight, about 60% to about 80% by weight, about 65% to about 80% by weight, about 65% to about 75% by weight, or about 68% to about 72% by weight of the total composition. In a preferred embodiment, the unsaturated free fatty acid is present in the pharmaceutical composition at a level of about 65% to about 75% by weight of the total composition. In a most preferred embodiment, the unsaturated free fatty acid is present in the pharmaceutical composition at a level of about 68% to about 72% by weight of the total composition. In a most preferred embodiment, the unsaturated free fatty acid is present in the pharmaceutical composition at a level of 68% to 72% by weight of the total composition.

The compositions according to the present invention comprise at least two emulsifiers. In an embodiment, the compositions according to the present invention comprise two emulsifiers. The at least two emulsifiers comprise at least a first emulsifier which has a HLB value greater than about 14 and at least a second emulsifier which has a HLB value less than about 6.

In an embodiment, the at least two emulsifiers is three emulsifiers. In a further embodiment, the three emulsifiers comprise a first emulsifier which has a HLB value greater than about 14, a second emulsifier which has a HLB value less than about 6 and a third emulsifier which has a HLB value in the range of about 8 to about 15.

HLB values are commonly used to define emulsifiers and/or surfactants and refer to the hydrophilic-lipophilic balance of the given compound. HLB values can be calculated according to the methods of Griffin [Griffin, J. Soc. Cosmetic Chem. (1949), 311-326; Griffin, J. Soc. Cosmetic Chem. (1954), 249-256] as follows:

HLB=20×(MW-H/MW-T)

wherein MW-H is the molecular weight of the hydrophilic portion of the compound and MW-T is the molecular weight of the total compound. For example, for the emulsifier PEG100 stearate, MW-H is the molecular weight of the ethylene glycol portions of the molecule which is 100×44 (MW ethylene oxide monomer=44 g/mol)=4400. Stearic acid has a molecular weight of 284.5 g/mol, so MW-T=4684.5. Therefore, the HLB value for PEG100 stearate is calculated at 18.8. Glycerol monooleate has a HLB value of 3.5. HLB values for a selection of components are listed in the table below.

| Material | Trade name | HLB |
|---|---|---|
| Oleic acid | | 1 |
| Glyceryl mono-/di-/tribehenate | Compritol ® 888 ATO | 2 |
| Glycerol monooleate | Peceol ® | 3.5 |
| PEG-6 glyceryl oleate | Labrafil ® M1944CS | 4 |
| Glycerol monolinoleate | Maisine ® 35-1 | 4 |
| Diethylene glycol monoethyl ether | Transcutol ®, Carbitol ® | 4.2 |
| Sorbitan monooleate | Span ® 80 | 4.3 |
| Triglycerol diisostearate | Plurol ® diisostearique | 4-5 |
| Propylene glycol monocaprylate | Capmul ® PG8 | 5-6 |
| Medium chain monoglycerides | Akoline ® MCM | 5-6 |
| Glyceryl mono-/dicaprylate | Capmul ® MCM | 5.5-6 |
| Polyglyceryl-3 dioleate | Plurol ® oleique CC497 | 6 |
| Propylene glycol monocaprylate | Capryol ® 90 | 6 |
| Polyglyceryl-6 distearate | Plurol ® stearic WL1009 | 9-10 |
| PEG-35 castor oil | Cremophor ® EL, Kolliphor ® EL | 12-14 |
| D-alpha-tocopheryl PEG-1000 | Vitamin E TPGS | 13 |
| succinate | | |
| Caprylocaproy1 PEG-8 glycerides | Labrasol ® | 14 |
| PEG-32 glyceryl laureate | Gelucire ® 44/14 | 14 |
| PEG-40 hydrogenated castor oil | Cremophor ® RH40, Kolliphor ® | 14-16 |
| | RH40 | |
| PEG-60 sorbitan monostearate | Tween ® 60 | 14.9 |
| PEG-80 sorbitan monooleate | Tween ® 80 | 15 |
| PEG-20 sorbitan monolaurate | Tween ® 20 | 16.7 |
| PEG-100 stearate | Myrj ™ S100 | 18.8 |

In an embodiment, the first emulsifier has a HLB value greater than 14, greater than about 15, greater than about 16, greater than about 16.5, greater than about 17 or greater than about 18. In a preferred embodiment, the first emulsifier has a HLB value greater than about 17, such as greater than 17. In a most preferred embodiment, the first emulsifier has a HLB value greater than about 16.5, such as greater than 18.

In an embodiment, the first emulsifier is a polyol ester. A polyol ester emulsifier, as used herein, refers to a non-ionic emulsifier which comprises a polymeric backbone having multiple hydroxyl groups, wherein at least one of those hydroxyl groups has been converted to ester groups. In an embodiment, the polyol ester is an ester of polyethylene glycol (PEG). In an embodiment, the PEG has an average molecular weight in the range 1000-9000 g/mol, such as 3000-6000, or 4000-5000 g/mol. In an embodiment the PEG comprises 20-200 ethylene glycol monomer units, such 50-150, or 80-120 ethylene glycol monomer units. In an embodiment the PEG comprises about 100 ethylene glycol monomer units. In an embodiment the ester is an ester of a fatty acid, such as an ester of capric acid, undecylic acid, lauric acid, tridecylic acid, myristic acid, pentadecylic acid, palmitic acid, margaric acid, stearic acid, nonadecylic acid or arachidic acid. In an embodiment the ester is an ester of stearic acid. In an embodiment the first emulsifier is a polyol stearate. In an embodiment, the first emulsifier is a PEG stearate. In a preferred embodiment, the first emulsifier is PEG 100 stearate.

In an embodiment, the first emulsifier is a polyethoxylated sorbitan ester. In an embodiment the ester is a monoester of polyethoxylated sorbitan. In an embodiment, the sorbitan is ethoxylated with 10-100 ethylene glycol monomer units, such as 20-100, or 20-80 ethylene glycol monomer units. In an embodiment, the sorbitan is ethoxylated with a total of 20, 60 or 80 ethylene glycol monomer units. In an embodiment the ester is an ester of a fatty acid, such as an ester of capric acid, undecylic acid, lauric acid, tridecylic acid, myristic acid, pentadecylic acid, palmitic acid, margaric acid, stearic acid, nonadecylic acid, oleic acid or arachidic acid. In an embodiment the ester is an ester of lauric acid, stearic acid or oleic acid. In an embodiment the first emulsifier is PEG-20 sorbitan monolaurate (polysorbate 20), PEG-60 sorbitan monostearate (polysorbate 20) or PEG-80 sorbitan monooleate (polysorbate 80). In a preferred embodiment, the first emulsifier is PEG-20 sorbitan monolaurate.

In an embodiment, the second emulsifier has a HLB value less than about 5.5, such as less than about 5, less than about 4.5, or less than about 4. In a preferred embodiment, the second emulsifier has a HLB value less than about 4.5, such as less than 4.5. In a most preferred embodiment, the second emulsifier has a HLB value less than about 4, such as less than 4.

In an embodiment, the second emulsifier is a monoglyceride. A monoglyceride emulsifier, as used herein, refers to a non-ionic emulsifier wherein a molecule of glycerol is linked to a fatty acid via an ester bond. It comprises glycerides where the fatty acid is attached to either a primary alcohol or a secondary alcohol site on glycerol. The fatty acid portion of the monoglyceride may be either saturated or unsaturated. In an embodiment, the monoglyceride comprises an ester of glycerol and a fatty acid selected from capric acid, undecylic acid, lauric acid, tridecylic acid, myristic acid, pentadecylic acid, palmitic acid, margaric acid, stearic acid, nonadecylic acid, arachidic acid, behenic acid, myristoleic acid, palmitoleic acid, sapienic acid, oleic acid, elaidic acid, vaccenic acid, linoleic acid and erucic acid. In an embodiment, the second emulsifier is selected from glycerol monostearate, glycerol monolaurate, glycerol monooleate and glycerol monolinoleate, or a mixture thereof. In a preferred embodiment, the second emulsifier is glycerol monooleate.

In an embodiment, the first emulsifier has a HLB value greater than about 15 and the second emulsifier has a HLB value less than about 5.5. In an embodiment, the first emulsifier has a HLB value greater than about 16 and the second emulsifier has a HLB value less than about 5. In an embodiment, the first emulsifier has a HLB value greater than about 16.5 and the second emulsifier has a HLB value less than about 4.5. In an embodiment, the first emulsifier has a HLB value greater than about 18 and the second emulsifier has a HLB value less than about 4.5. In an embodiment, the first emulsifier has a HLB value greater than about 18 and the second emulsifier has a HLB value less than about 4. In an embodiment, the first emulsifier has a HLB value greater than about 16.5 and the second emulsifier has a HLB value less than about 4.

It has been surprisingly found that solid and/or semi-solid emulsifiers can be incorporated into self-emulsifying compositions and give beneficial API dissolution profiles. Therefore, in an embodiment, the first emulsifier is a solid at room temperature. In an embodiment, the second emulsifier is a semi-solid at room temperature. In an embodiment, the first emulsifier is a solid at room temperature and the second emulsifier is a semi-solid at room temperature. The meaning of the terms ‘solid’ and ‘semi-solid’ will be apparent to person of skill in the art of pharmaceutical formulations. Nevertheless, it will be understood that a liquid is a material that flows in response to an external force, whereas a solid is a material that does not flow in response to an external force. A semi-solid manifests a degree of both solid and liquid attributes.

It has been found that a synergistic interaction exists between the two emulsifiers, whereby the compositions containing at least two emulsifiers provide superior active pharmaceutical ingredient (API) release profiles, than comparative compositions containing only the single emulsifier. In other words, compositions according to the present invention comprising the at least two emulsifiers, may typically provide faster API release profiles than comparative compositions containing only one of the at least two emulsifiers, even if the total emulsifier content of the compositions expressed as % by weight is the same. In an embodiment, the wt/wt ratio of second emulsifier to first emulsifier present in the composition is between about 1:10 and about 10:1, such as between about 1:5 and about 5:1, between about 1:3 and about 3:1, between about 1:1 and about 5:1, between about 1:1 and about 3:1, between about 1:1 and about 2:1, between about 1:2 and about 2:1, between about 1:1.5 and about 2:1, between about 1:1.3 and about 1:1.1 (such as about 1:1.2), or between about 1.4:1 and 1.6:1 (such as about 1.5:1). In a preferred embodiment, the wt/wt ratio of second emulsifier to first emulsifier present in the composition is between about 1:1 and about 2:1, such as between about 1.2:1 and 1.8:1, between about 1.1:1 and 1.6:1 or between about 1.4:1 and 1.6:1. In a most preferred embodiment, the wt/wt ratio of second emulsifier to first emulsifier present in the composition is about 1.5:1. In a most preferred embodiment, the wt/wt ratio of second emulsifier to first emulsifier present in the composition is about 1.2:1.

In an alternative embodiment, the wt/wt ratio of second emulsifier to first emulsifier present in the composition is between about 1:2 and about 1:1, such as between about 1:1.5 and 1:1, or between about 1:1.3 and 1:1.1. In a further embodiment, the wt/wt ratio of second emulsifier to first emulsifier present in the composition is about 1:1.2. In a yet further embodiment, the wt/wt ratio of second emulsifier to first emulsifier present in the composition is 1:1.2.

In an embodiment, the first emulsifier is present in the pharmaceutical composition at about 1% to about 20% by weight of the total pharmaceutical composition, such as about 1% to about 10% by weight, about 1% to about 5% by weight, about 3% to about 7% by weight, about 3% to about 6% by weight, about 3% to about 5% by weight (such as about 4% by weight), about 4% to about 5% by weight (such as about 4.3% by weight), or about 5% to about 6% by weight (such as about 5.5% by weight) of the total composition. In a preferred embodiment, the first emulsifier is present in the pharmaceutical composition at a level of about 1% to about 5% by weight of the total composition. In a preferred embodiment, the first emulsifier is present in the pharmaceutical composition at a level of about 2% to about 4% by weight of the total composition. In a most preferred embodiment, the first emulsifier is present in the pharmaceutical composition at a level of about 3% to about 5% by weight of the total composition. In a most preferred embodiment, the first emulsifier is present in the pharmaceutical composition at a level of about 4% by weight of the total composition. In an embodiment, the first emulsifier is present in the pharmaceutical composition at a level of about 5% to about 15% by weight (such as about 7% to about 14% by weight, about 8% to about 13% by weight, or about 9% to about 13% by weight) of the total composition. In an embodiment, the first emulsifier is present in the pharmaceutical composition at a level of about 10% to about 12% by weight of the total composition.

In an alternative embodiment, the first emulsifier is present in the composition at a level of about 4% to about 7% by weight of the total composition. In a further embodiment, the first emulsifier is present in the pharmaceutical composition at a level of about 5% to about 6% by weight of the total composition. In a yet further embodiment, the first emulsifier is present in the pharmaceutical composition at a level of about 5.5% by weight of the total composition.

In an embodiment, the second emulsifier is present in the pharmaceutical composition at about 1% to about 20% by weight of the total pharmaceutical composition, such as about 1% to about 10/o by weight, about 2% to about 8% by weight, about 4% to about 7% by weight, about 4% to about 6% by weight (such as about 5% by weight), about 5% to about 7% by weight (such as about 6% by weight), or about 4% to about 5% by weight (such as about 4.5% by weight) of the total composition. In a preferred embodiment, the second emulsifier is present in the pharmaceutical composition at a level of about 1% to about 7% by weight of the total composition. In a preferred embodiment, the second emulsifier is present in the pharmaceutical composition at a level of about 3% to about 6% by weight of the total composition. In a most preferred embodiment, the second emulsifier is present in the pharmaceutical composition at a level of about 5% to about 7% by weight of the total composition. In a most preferred embodiment, the second emulsifier is present in the pharmaceutical composition at a level of about 6% by weight of the total composition. In a most preferred embodiment, the second emulsifier is present in the pharmaceutical composition at a level of about 5% by weight of the total composition.

In an embodiment, the second emulsifier is present in the pharmaceutical composition at a level of about 5% to about 20% by weight (such as about 7% to about 18% by weight, about 9% to about 16% by weight, or about 10% to about 15% by weight) of the total composition. In an embodiment, the second emulsifier is present in the pharmaceutical composition at a level of about 12% to about 15% by weight of the total composition. In an embodiment, the second emulsifier is present in the pharmaceutical composition at a level of about 13% to about 14% by weight of the total composition.

In an alternative embodiment, the second emulsifier is present in the composition at a level of about 3% to about 6% by weight of the total composition. In a further embodiment, the second emulsifier is present in the pharmaceutical composition at a level of about 4% to about 5% by weight of the total composition. In a yet further embodiment, the second emulsifier is present in the pharmaceutical composition at a level of about 4.5% by weight of the total composition.

In certain embodiments of the present invention it may be desirable or beneficial to have more than two emulsifiers. In an embodiment, the compositions according to the present invention comprise three emulsifiers. In a further embodiment, the three emulsifiers comprise a first emulsifier which has a HLB value greater than about 14, a second emulsifier which has a HLB value less than about 6 and a third emulsifier which has a HLB value in the range of about 8 to about 15.

In an embodiment, the third emulsifier has a HLB value in the range 10 to 15, such as 11 to 15, 12 to 15, 13 to 15, 11 to 14 or 12 to 14.

In an embodiment, the third emulsifier is polyoxyl castor oil derivative. In an embodiment the third emulsifier is PEG 30 castor oil, PEG 35 castor oil, PEG 40 castor oil or PEG 60 castor oil. In a preferred embodiment, third emulsifier is PEG 35 castor oil.

In an embodiment, the third emulsifier is present in the pharmaceutical composition at about 1% to about 10% by weight of the total pharmaceutical composition, such as about 2% to about 8% by weight, about 3% to about 7% by weight, about 4% to about 6% by weight, or about 5% by weight of the total composition.

While SEDDS formulations typically have high total emulsifier contents (approximately 30% to 60% w/w of the total composition), in an aspect of the present invention it has been surprisingly discovered that with the combination of unsaturated free fatty acid and emulsifiers disclosed herein, SEDDS can be obtained which have good API dissolution profiles, but with only relatively low total emulsifier contents. The self-emulsifying pharmaceutical compositions of the present invention have a total emulsifier content of less than 30% by weight of the total composition. In an embodiment, the total emulsifier content is less than 25% by weight, such as less than 20% by weight, less than 15% by weight, or less than 12% by weight of the total composition. In an embodiment, the total emulsifier content is 2% to 20% by weight of the total composition, 2.5% to 15% by weight, 5% to 15% by weight, 8% to 12%, 10% to 20%, 12% to 20%, 10% to 18%, 12% to 18%, 12% to 16%, 13% to 20%, 14% to 20%, 15% to 20%, 13% to 25%, 14% to 25%, 15% to 25%, 20% to 25%, 20% to 26%, 20% to 27%, 20% to 28% or 20% to 29% by weight of the total composition. In one embodiment, the total emulsifier content is 8% to 12% by weight of the total composition. In one embodiment, the total emulsifier content is about 10% by weight, such as 10% by weight, of the total composition. In one embodiment, the total emulsifier content is about 14 to 15% by weight, such as about 14.3% by weight, of the total composition. In one embodiment, the total emulsifier content is about 28 to 29.9% by weight, such as about 29 to 29.9% by weight, or about 29.5% by weight, of the total composition.

In an embodiment, the first emulsifier is present in the pharmaceutical composition at about 3% to about 7% by weight (such as about 4% to about 5% by weight) of the total pharmaceutical composition, the second emulsifier is present in the pharmaceutical composition at about 4% to about 6% by weight of the total pharmaceutical composition and the third emulsifier is present in the pharmaceutical composition at about 4% to about 6% by weight of the total pharmaceutical composition. In an alternative embodiment, the first emulsifier is present in the pharmaceutical composition at about 9% to about 13% by weight (such as about 10% to about 12% by weight) of the total pharmaceutical composition, the second emulsifier is present in the pharmaceutical composition at about 12% to about 15% by weight (such as about 13% to about 14% by weight) of the total pharmaceutical composition and the third emulsifier is present in the pharmaceutical composition at about 4% to about 6% by weight of the total pharmaceutical composition, provided that the total emulsifier content is less than 30% by weight of the total composition.

The compositions according to the present invention are particularly suited to solubilising poorly soluble APIs. They are particularly suited to solubilising lipophilic APIs which are poorly soluble in aqueous environments. For example, lopinavir and ritonavir are practically insoluble in water and have predicted aqueous solubilities of 1.9 μg/ml and 1.3 μg/ml respectively [www.drugbank.ca]. Therefore, in an embodiment, the at least one active pharmaceutical ingredient has an aqueous solubility (either measured or predicted) of less than 1 mg/ml, such as less than 0.1 mg/ml, or less than 0.01 mg/ml (less than 10 μg/ml).

The lipophilicity or hydrophobicity of an API can be determined by routine methods well-known to those of skill in the art. For example, log P can be determined from the partitioning of an API between octanol and water. Log P values may range from approximately −10 to +10, but most marketed APIs fall within the log P range of −2 to +6 and in particular 0 to +5, wherein the higher the log P value the more lipophilic the API. For example, the lipophilic APIs lopinavir and ritonavir have calculated log P values [ACD/Labs] of 6.26 and 5.28 respectively.

In an embodiment, the at least one active pharmaceutical ingredient has a log P value, or a calculated log P (clog P) value, greater than 4, such as greater than 4.5, greater than 5.0, or greater than 5.5.

In one embodiment, the at least one active pharmaceutical ingredient is a solid at room temperature. In one embodiment, the at least one active pharmaceutical ingredient is synthetically prepared. In one embodiment, the at least one active pharmaceutical ingredient is not a fatty acid (free or bound state).

In one embodiment, the at least one active pharmaceutical ingredient (lopinavir) is present in a dissolved state in the pharmaceutical composition. In another embodiment, the at least one active pharmaceutical ingredient (lopinavir) is present in a dispersed state in the pharmaceutical composition. In another embodiment, an amount of the least one active pharmaceutical ingredient (lopinavir) is present in a dispersed state and an amount is present in a dissolved state in the pharmaceutical composition. It will be apparent to the skilled person that the active pharmaceutical ingredient is dissolved or dispersed within the pharmaceutical composition by the use of techniques such as optical microscopy using polarised light filters, differential scanning calorimetry or micro FTIR. For example, a placebo pharmaceutical composition (i.e., a composition containing no active pharmaceutical ingredients) can be spiked with a crystalline active pharmaceutical ingredient. When viewed under an optical microscope using polarised light filters, the crystalline active pharmaceutical ingredient will exhibit birefringence. Thus, the spiked placebo composition can be used as a comparison standard in order to confirm there is no crystalline active pharmaceutical ingredient in the pharmaceutical composition, and thereby demonstrating the active pharmaceutical ingredient is dissolved within the pharmaceutical composition. Alternatively, and/or additionally, micro FTIR can be used to confirm the active pharmaceutical ingredient (lopinavir) is dissolved in the pharmaceutical composition. In this case, spectra obtained for a pharmaceutical composition spiked with the active pharmaceutical ingredient and for the pharmaceutical composition can be compared and used to demonstrate the active pharmaceutical ingredient (lopinavir) is dissolved in the pharmaceutical composition.

In one embodiment, the at least one active pharmaceutical ingredient (lopinavir and/or ritonavir) is stable within the pharmaceutical composition. The compositions of the invention are particularly suitable for active pharmaceutical ingredients used in the composition that are prone to chemical or physical degradation. In one embodiment, the active pharmaceutical ingredient (lopinavir and/or ritonavir) used in the composition is prone to degradation due to hydrolysis. In one embodiment, the active pharmaceutical ingredient (lopinavir and/or ritonavir) used in the composition is prone to degradation due to oxidation. In one embodiment, the active pharmaceutical ingredient (lopinavir) used in the composition is prone to degradation which is accelerated by heat. Due to the ambient temperature processing used to manufacture the compositions of the present invention, the compositions typically have a reduced API (lopinavir) impurity burden compared to compositions which require heat (e.g. >40° C.) during their preparation. In an embodiment, the pharmaceutical composition comprises lopinavir-derived impurities totalling no more than 0.5% by weight, such as less than 0.45%, less than 0.40%, less than 0.35%, less than 0.30%, or less than 0.25% by weight.

In an embodiment, the pharmaceutical composition comprises ritonavir-derived impurities totalling no more than 5% by weight, such as less than 4.5%, less than 4.0% or less than 3.5% by weight.

In an embodiment, the pharmaceutical composition comprises lopinavir-derived impurities totalling no more than 0.5% by weight (such as less than 0.45%, less than 0.40%, less than 0.35%, less than 0.30%, or less than 0.25% by weight) and ritonavir-derived impurities totalling no more than 5% by weight (such as less than 4.5%, less than 4.0% or less than 3.5% by weight). In an embodiment, the pharmaceutical composition comprises lopinavir-derived impurities totalling less than 0.25% by weight and ritonavir-derived impurities totalling less than 3.5% by weight.

In one embodiment, the active pharmaceutical ingredient (e.g. ritonavir) used in the composition is prone to physical form changes, e.g. solid-state polymorphic transitions.

In one embodiment, the at least one active pharmaceutical ingredient (lopinavir) is stable within the pharmaceutical composition for at least 3 months, such as at least 6 months, such as at least 9 months, such as at least 12 months, such as at least 18 months, such as at least 24 months, or such as at least 36 months, at a temperature of 5° C., 25° C., 30° C. or at 45° C., and/or at a relative humidity of 60%, 65%, or 75% RH. In an embodiment, the at least one active pharmaceutical ingredient (lopinavir) is stable within the pharmaceutical composition for at least 3 months, such as at least 6 months, such as at least 9 months, such as at least 12 months, such as at least 18 months, such as at least 24 months, or such as at least 36 months, at a temperature of 5° C.

In one embodiment, the at least one active pharmaceutical ingredient (lopinavir) is stable within the pharmaceutical composition during a process to manufacture the pharmaceutical composition. In one embodiment, the at least one active pharmaceutical ingredient (lopinavir) is stable within the pharmaceutical composition during a process to manufacture the pharmaceutical composition wherein the process is performed at room temperature.

In one embodiment, the at least one active pharmaceutical ingredient has a solubility measured at ambient temperature of at least 1% w/v in the unsaturated free fatty acid, such as at least 5% w/v in the unsaturated free fatty acid, such as at least 10% w/v in the unsaturated free fatty acid, such as at least 12% w/v in the unsaturated free fatty acid, such as at least 15% w/v in the unsaturated free fatty acid, or such as at least 18% w/v in the unsaturated free fatty acid.

In one embodiment, the at least one active pharmaceutical ingredient is present in the pharmaceutical composition at a level between about 0.001% and about 50% by weight of the total composition weight, such as between about 0.01% and about 50% by weight, between about 0.001% and about 5% by weight, between about 0.1% and about 25% by weight, between about 0.5% and about 15% by weight, between about 0.5% and about 10% by weight, between about 0.5% and about 5% by weight, between about 0.5% and about 2.5% by weight, between about 1.0% and about 2.5% by weight, between about 1.0% and about 2.0% by weight, between about 1.2% and about 1.8% by weight, or between about 1.3% and about 1.7% by weight of the total composition weight.

In an embodiment, the at least one active pharmaceutical ingredient is present in the pharmaceutical composition at a level of about 0.5% by weight, about 0.6% by weight, about 0.7% by weight, about 0.8% by weight, about 0.9% by weight, about 1.0% by weight, about 1.1% by weight, about 1.2% by weight, about 1.3% by weight, about 1.4% by weight, about 1.5% by weight, about 1.6% by weight, about 1.7% by weight, about 1.8% by weight, about 1.9% by weight, or about 2.0% by weight of the total composition weight.

In an embodiment, the at least one active pharmaceutical ingredient (lopinavir) is present in the pharmaceutical composition at a level between about 1% and about 50% by weight of the total composition weight, such as between about 5% and about 50% by weight, between about 5% and about 25% by weight, between about 5% and about 20% by weight, between about 10% and about 25% by weight, between about 10% and about 20% by weight, between about 11% and about 19% by weight, between about 12% and about 20% by weight, between about 14% and about 20% by weight, between about 15% and about 20% by weight, or between about 15% and about 18% by weight, or between about 16% and about 19% by weight of the total composition weight.

In an embodiment, the at least one active pharmaceutical ingredient (lopinavir) is present in the pharmaceutical composition at a level of about 10% by weight, about 10.5% by weight, about 11% by weight, about 11.5% by weight, about 11.5% by weight, about 12% by weight, about 12.5% by weight, about 13% by weight, about 13.5% by weight, about 14% by weight, about 14.5% by weight, about 15% by weight, about 15.5% by weight, about 16% by weight, about 16.5% by weight, about 17% by weight, about 17.5% by weight, about 18% by weight, about 18.5% by weight, about 19% by weight, about 19.5% by weight or about 20% by weight of the total composition weight.

In an embodiment, the pharmaceutical composition according to the present invention comprises two or more active pharmaceutical ingredients. In an embodiment, the pharmaceutical composition according to the present invention comprises two active pharmaceutical ingredients. In an embodiment, the two or more active pharmaceutical ingredients are present in the pharmaceutical composition at a combined level of about 5% to about 30% by weight, about 5% to about 25% by weight, about 10% to about 30% by weight, about 10% to about 25% by weight, about 15% to about 30% by weight, about 15% to about 25% by weight, about 16% to about 24% by weight, about 17% to about 23% by weight, about 17% to about 21% by weight, about 18% to about 22% by weight, or about 18% to about 20% by weight, or about 18% to about 21% by weight of the total composition weight.

In one embodiment, the at least one active pharmaceutical ingredient is classified as a Biopharmaceutics Classification System (BCS) Class II or a BCS Class IV active pharmaceutical ingredient. A BCS class II active pharmaceutical ingredient is classed as an active ingredient having a high permeability and a low solubility. A BCS class IV active pharmaceutical ingredient is classed as an active ingredient having a low permeability and a low solubility. According to ICH guidelines (ICH guideline M9 on biopharmaceutics classification system based biowaivers; 6^{th} August 2018) a drug substance is classified as highly soluble if the highest single therapeutic dose is completely soluble in 250 ml or less of aqueous media over the pH range of 1.2-6.8 at 37±1° C. The assessment of permeability should preferentially be based on the extent of absorption derived from human pharmacokinetic studies, e.g., absolute bioavailability or mass balance. High permeability can be concluded when the absolute bioavailability is ≥85%.

In one embodiment, the at least one active pharmaceutical ingredient is selected from a protease inhibitor, a retinoid, a vitamin D analog, an antileprosy active pharmaceutical ingredient, a calcineurin inhibitor, a cannabinoid, a 5 alpha-reductase inhibitor, an androgen receptor inhibitor, a peroxisome proliferator activated receptor activator, an antihistamine, a chloride channel activator, a tyrosine kinase inhibitor, a hormone, a protease inhibitor, and a mTOR kinase inhibitor.

In one embodiment, the at least one active pharmaceutical ingredient is selected from abacavir, acitretin, alitretinoin, efavirenz, enfuvirtide, estradiol, nevirapine, ritonavir, lopinavir, tenofovir, adefovir, entecavir, ribavirin, acyclovir, famciclovir, penciclovir, valacyclovir, cidofovir, ganciclovir, valganciclovir, oseltamivir, zanamivir, amprenavir, bexarotene, calcifediol, calcitriol, clofazimine, cyclosporin A, doxercalciferol, dronabinol, dutasteride, enzalutamide, fenofibrate, isotretinoin, loratadine, lubiprostone, nintedanib, paricalcitol, progesterone, saquinavir, sirolimus, tipranavir, tretinoin, atorvastatin, carvedilol, itraconazole, ketoprofen, and simvastatin.

In one embodiment, the at least one active pharmaceutical ingredient is selected from abacavir, efavirenz, enfuvirtide, estradiol, nevirapine, ritonavir, lopinavir, tenofovir, adefovir, entecavir, ribavirin, acyclovir, famciclovir, penciclovir, valacyclovir, cidofovir, ganciclovir, valganciclovir, oseltamivir and zanamivir.

In one embodiment, the pharmaceutical composition further comprises an active pharmaceutical ingredient synergist. In one embodiment, the active pharmaceutical ingredient synergist is a HIV protease enzyme inhibitor.

In one embodiment, the at least one active pharmaceutical ingredient is a HIV protease enzyme inhibitor. In one embodiment, the HIV protease enzyme inhibitor is selected from lopinavir and ritonavir.

Lopinavir (CAS #192725-17-0) is a protease inhibitor chemically designated as [1S-[1R*(R*),3R*,4R*]]-N-[4[[(2,6-dimethylphenoxy)acetyl]amino]-3-hydroxy-5-phenyl-1-(phenylmethyl)pentyl]-tetrahydro-α-(1-methylethyl)-2-oxo-1(2H)-pyrimidineacetamide (IUPAC name=(2S)-N-[(2S,4S,5S)-5-[[2-(2,6-dimethylphenoxy)acetyl]amino]-4-hydroxy-1,6-diphenyl-hexan-2-yl]-3-methyl-2-(2-oxo-1,3-diazinan-1-yl)butanamide). It has the molecular formula C₃₇H₄₈N₄O₅ and a molecular weight of 628.80.

Ritonavir (CAS #155213-67-5) is a protease inhibitor chemically designated as 2,4,7,12-Tetraazatridecan-13-oic acid, 10-hydroxy-2-methyl-5-(1-methylethyl)-1-[2-(1-methylethyl)-4-thiazolyl]-3,6-dioxo-8,11-bis(phenylmethyl)-5-thiazolylmethyl ester [5S-(5R*,8R*,10R*,11R*)](IUPAC name=1,3-thiazol-5-ylmethyl N-[(2S,3S,5S)-3-hydroxy-5-[[(2S)-3-methyl-2-[[methyl-[(2-propan-2-yl-1,3-thiazol-4-yl)methyl]carbamoyl]amino]butanoyl]amino]-1,6-diphenylhexan-2-yl]carbamate). It has the molecular formula C₃₇H₄₈N₆O₅S₂ and a molecular weight of 720.94.

In one embodiment, the pharmaceutical composition comprises lopinavir and ritonavir.

In one embodiment, the molar ratio of lopinavir to ritonavir present in the composition is between about 1:10 and about 18:1, such as between about 1:10 and about 15:1, such as between about 1:5 and about 15:1, such as between about 1:1 and about 15:1, such as between about 2:1 and about 15:1, such as between about 4:1 and about 15:1, such as between about 8:1 and about 14:1, such as between about 9:1 and about 14:1, such as between about 10:1 and about 14:1, such as between 10.5:1 and about 18:1, such as between 10.5:1 and 18:1, such as between about 10.5:1 and about 14:1, such as between about 11:1 to about 13:1, such as between about 11.5 and about 17:1, such as between about 11.5:1 and about 16.0:1, such as between about 11.5:1 and about 15:1, such as about 14.5:1, such as 14.5:1, such as about 14:1, such as 14:1, such as about 13.8:1, such as 13.8:1, such as about 13.75:1, such as 13.75:1, such as about 13.5:1, such as 13.5:1, such as about 13:1, such as 13:1, such as about 12.5:1, such as 12.5:1, such as about 12:1, such as 12:1, such as about 11.75:1, such as 11.75:1, such as about 9:1, such as 9:1, such as about 5:1, such as 5:1, such as about 4.6:1, or such as 4.6:1. In a preferred embodiment, the molar ratio of lopinavir to ritonavir present in the composition is about 13.8:1, such as 13.8:1.

In one embodiment, the wt/wt ratio of lopinavir to ritonavir present in the composition is between about 1:10 and about 18:1, such as between about 1:10 and about 15:1, such as between about 1:5 and about 15:1, such as between about 1:1 and about 15:1, such as between about 2:1 and about 15:1, such as between about 4:1 and about 15:1, such as between about 8:1 and about 14:1, such as between about 9:1 and about 14:1, such as between about 10:1 and about 14:1, such as between 10.5:1 and about 18:1, such as between 10.5:1 and 18:1, such as between about 10.5:1 and about 14:1, such as between about 11:1 to about 13:1, such as between about 11.5 and about 17:1, such as between about 11.5:1 and about 16.0:1, such as between about 11.5:1 and about 15:1, such as about 14.5:1, such as 14.5:1, such as about 14:1, such as 14:1, such as about 13.8:1, such as 13.8:1, such as about 13.75:1, such as 13.75:1, such as about 13.5:1, such as 13.5:1, such as about 13:1, such as 13:1, such as about 12.5:1, such as 12.5:1, such as about 12:1, such as 12:1, such as about 11.75:1, such as 11.75:1, such as about 11.5:1, such as 11.5:1, such as about 11.25:1, such as 11.25:1, or such as about 11:1, such as 11:1. In a preferred embodiment, the wt/wt ratio of lopinavir to ritonavir present in the composition is about 12:1, such as 12:1.

In an embodiment, lopinavir is present in the pharmaceutical composition at a level between about 1% and about 50% by weight of the total composition weight, such as between about 5% and about 50% by weight, between about 5% and about 25% by weight, between about 5% and about 20% by weight, between about 10% and about 25% by weight, between about 10% and about 20% by weight, between about 11% and about 19% by weight, between about 12% and about 20% by weight, between about 14% and about 20% by weight, between about 15% and about 20% by weight, or between about 16% and about 19% by weight of the total composition weight.

In an embodiment, lopinavir is present in the pharmaceutical composition at a level of about 10% by weight, about 10.5% by weight, about 11% by weight, about 11.5% by weight, about 11.5% by weight, about 12% by weight, about 12.5% by weight, about 13% by weight, about 13.5% by weight, about 14% by weight, about 14.5% by weight, about 15% by weight, about 15.5% by weight, about 16% by weight, about 16.5% by weight, about 17% by weight, about 17.5% by weight, about 18% by weight, about 18.5% by weight, about 19% by weight, about 19.5% by weight, or about 20% by weight of the total composition weight.

In one embodiment, ritonavir is present in the pharmaceutical composition at a level between about 0.001% and about 50% by weight of the total composition weight, such as between about 0.01% and about 50% by weight, between about 0.001% and about 5% by weight, between about 0.1% and about 25% by weight, between about 0.5% and about 15% by weight, between about 0.5% and about 10% by weight, between about 0.5% and about 5% by weight, between about 0.5% and about 2.5% by weight, between about 1.0% and about 2.5% by weight, between about 1.0% and about 2.0% by weight, between about 1.2% and about 1.8% by weight, or between about 1.3% and about 1.7% by weight of the total composition weight.

In an embodiment, ritonavir is present in the pharmaceutical composition at a level of about 0.5% by weight, about 0.6% by weight, about 0.7% by weight, about 0.8% by weight, about 0.9% by weight, about 1.0% by weight, about 1.1% by weight, about 1.2% by weight, about 1.3% by weight, about 1.4% by weight, about 1.5% by weight, about 1.6% by weight, about 1.7% by weight, about 1.8% by weight, about 1.9% by weight, or about 2.0% by weight of the total composition weight.

Optionally, additional excipients may be included in the compositions according to the present invention, providing that inclusion of such excipients does not unacceptably impact the ability of the composition to be self-emulsifying.

In one embodiment, the pharmaceutical composition further comprises an antioxidant. In one embodiment, the antioxidant is butylated hydroxyanisole (BHA), tert-butylhydroquinone (TBHQ) or butylated hydroxytoluene (BHT). In a preferred embodiment, the antioxidant is butylated hydroxytoluene. In one embodiment, the antioxidant is present in the pharmaceutical composition at about 0.05 to about 0.5% by weight of the total pharmaceutical composition by weight, such as about 0.05 to about 0.15% by weight, such as about 0.1 to about 0.3% by weight, such as about 0.2% by weight, such as 0.2% by weight, such as about 0.1% by weight, or such as 0.1% by weight.

In one embodiment, the pharmaceutical composition is an anhydrous pharmaceutical composition. In one embodiment, the pharmaceutical composition comprises less than 5% by weight of water of the total pharmaceutical composition weight, such as less than 1% by weight, less than 0.5% by weight, less than 0.1% by weight, or less than 0.05% by weight of water of the total pharmaceutical composition weight. In one embodiment, the pharmaceutical composition is substantially free of water. In one embodiment, the pharmaceutical composition is entirely free of water.

In one embodiment, the pharmaceutical composition according to the present invention further comprises a thickener. In an alternative embodiment, the pharmaceutical composition according to the present invention does not comprise a thickener. A thickener is an excipient which, when added to a mixture, increases the viscosity of the mixture. In one embodiment, the thickener is selected from mono di glyceride, ceresin wax, and hydrogenated vegetable oil or a combination thereof. In one embodiment, pharmaceutical composition according to the present invention does not comprise mono di glyceride, ceresin wax, or hydrogenated vegetable oil.

In one embodiment, the pharmaceutical composition according to the present invention further comprises a stiffening agent. In an alternative embodiment, the pharmaceutical composition according to the present invention does not comprise a stiffening agent. A stiffening agent is an excipient used to stiffen a composition so that the anhydrous composition is a semi-solid at room temperature. Typically, the stiffening agent may be a saturated free fatty acid, such as a C₁₀-C₃₈ saturated free fatty acid, such as a C₁₆-C₂₂ saturated free fatty acid. A saturated free fatty acid, is a free fatty acid (i.e., the fatty acid is not bound to another molecule, such as glycerol) wherein there are no double bonds between the carbon atoms in the fatty acid. In one embodiment, the stiffening agent is selected from capric acid, undecylic acid, lauric acid, tridecylic acid, myristic acid, pentadecylic acid, palmitic acid, margaric acid, stearic acid, nonadecylic acid, arachidic acid, heneicosylic acid, behenic acid, tricosylic acid, lignoceric acid, pentacosylic acid, cerotic acid, heptacosylic acid, montanic acid, nonacosylic acid, melissic acid, henatriacontylic acid, lacceroic acid, psyllic acid, geddic acid, ceroplastic acid, hexatriacontylic acid, heptatriacontanoic acid and octatriacontanoic acid. In an embodiment, the pharmaceutical composition according to the present invention does not comprise stearic acid.

In an embodiment, the pharmaceutical composition according to the present invention is liquid or semi-solid at room temperature. Preferably, the pharmaceutical composition according to the present invention is liquid at room temperature. Viscosity of the composition can be determined by measuring the dynamic viscosity for predominantly liquid compositions, or the complex viscosity for compositions having more of a semi-solid character.

Dynamic viscosity may be determined according to the method described in Example 3. In an embodiment, the pharmaceutical composition according to the present invention has a dynamic viscosity at 25° C. of less than 500 cP.s., such as less than 400 cP.s., less than 300 cP.s., or less than 200 cP.s. In an embodiment, the pharmaceutical composition according to the present invention has a dynamic viscosity at 25° C. of 10 to 500 cP.s., such as 25 to 400 cP.s., 50 to 300 cP.s., or 100 to 200 cP.s.

Complex viscosity is defined as the frequency-dependent viscosity function determined for a viscoelastic fluid by subjecting it to oscillatory shear stress. It may be determined according to the method described in Example 4. In an embodiment, the pharmaceutical composition according to the present invention has a complex viscosity of less than 1000 cP.s., such as less than 800 cP.s., less than 600 cP.s., less than 400 cP.s., or less than 200 cP.s., when the complex viscosity is measured at an angular frequency of 0.1 rad/s.

In one embodiment, the pharmaceutical composition according to the present invention comprises a muco-adhesive agent. In an alternative embodiment, the pharmaceutical composition according to the present invention does not comprise a muco-adhesive agent. A muco-adhesive agent attracts water/moisture at a site of application of the composition and alters the physical properties of the composition. A resulting muco-adhesive composition exhibits greater adhesiveness and/or tackiness. Typically, a muco-adhesive agent is a cellulose ether, such as methylcellulose, ethylcellulose or hydroxypropylmethylcellulose (also known as hypromellose). In an embodiment, the pharmaceutical composition according to the present invention does not comprise hydroxypropylmethylcellulose.

In an embodiment, the pharmaceutical composition according to the present invention does not comprise one or more of a thickener, a stiffening agent or a muco-adhesive agent.

In an embodiment, the pharmaceutical composition according to the present invention does not comprise an alcohol solvent, such as ethanol or glycol (e.g. propylene glycol). In an embodiment, the pharmaceutical composition according to the present invention does not comprise propylene glycol.

In one embodiment, there is provided a self-emulsifying pharmaceutical composition comprising:
a. an unsaturated free fatty acid;
b. a first emulsifier having a HLB value greater than about 14;
c. a second emulsifier having a HLB value less than about 6; and
d. lopinavir;
   wherein the total emulsifier content is less than 30% by weight of the total composition.

In one embodiment, there is provided a self-emulsifying pharmaceutical composition comprising:
a. an unsaturated free fatty acid;
b. a first emulsifier having a HLB value greater than about 14;
c. a second emulsifier having a HLB value less than about 6; and
d. lopinavir;
   wherein the total emulsifier content is less than 30% by weight of the total composition and the composition is liquid at room temperature.

In one embodiment, there is provided a self-emulsifying pharmaceutical composition comprising:
a. an unsaturated free fatty acid;
b. a first emulsifier having a HLB value greater than about 14;
c. a second emulsifier having a HLB value less than about 6; and
d. lopinavir;
   wherein the total emulsifier content is less than 30% by weight of the total composition and the composition does not comprise a thickener (such as mono/di glycerides, white ceresin wax or hydrogenated vegetable oil) or a stiffening agent (such as stearic acid).

In one embodiment, there is provided a self-emulsifying pharmaceutical composition comprising:
a. an unsaturated free fatty acid;
b. a first emulsifier having a HLB value greater than about 14;
c. a second emulsifier having a HLB value less than about 6; and
d. lopinavir;
   wherein the total emulsifier content is less than 30% by weight of the total composition and the composition does not comprise a muco-adhesive agent (such as hypromellose).

In one embodiment, there is provided a self-emulsifying pharmaceutical composition comprising:
a. oleic acid;
b. a first emulsifier having a HLB value greater than about 14;
c. a second emulsifier having a HLB value less than about 6; and
d. lopinavir;
   wherein the total emulsifier content is less than 30% by weight of the total composition.

In one embodiment, there is provided a self-emulsifying pharmaceutical composition comprising:
a. oleic acid;
b. a first emulsifier having a HLB value greater than about 16;
c. a second emulsifier having a HLB value less than about 5; and
d. lopinavir;
   wherein the total emulsifier content is less than 30% by weight of the total composition.

In one embodiment, there is provided a self-emulsifying pharmaceutical composition comprising:
a. oleic acid;
b. a first emulsifier having a HLB value greater than about 17;
c. a second emulsifier having a HLB value less than about 4; and
d. lopinavir;
   wherein the total emulsifier content is less than 30% by weight of the total composition.

In one embodiment, there is provided a self-emulsifying pharmaceutical composition comprising:
a. oleic acid;
b. a first emulsifier having a HLB value greater than about 16;
c. a second emulsifier having a HLB value less than about 5; and
d. lopinavir and ritonavir;
   wherein the total emulsifier content is less than 30% by weight of the total composition.

In one embodiment, there is provided a self-emulsifying pharmaceutical composition comprising:
a. oleic acid;
b. a first emulsifier having a HLB value greater than about 17;
c. a second emulsifier having a HLB value less than about 4; and
d. lopinavir and ritonavir;
   wherein the total emulsifier content is less than 20% by weight of the total composition.

In one embodiment, there is provided a self-emulsifying pharmaceutical composition comprising:
a. oleic acid;
b. a first emulsifier having a HLB value greater than about 17;
c. a second emulsifier having a HLB value less than about 4; and
d. lopinavir and ritonavir;
   wherein the total emulsifier content is less than 15% by weight of the total composition.

In one embodiment, there is provided a self-emulsifying pharmaceutical composition comprising:
a. an unsaturated free fatty acid;
b. a polyol ester (such as a polyol stearate, for example PEG100 stearate) emulsifier having a HLB value greater than about 15;
c. a second emulsifier having a HLB value less than about 5; and
d. lopinavir;
   wherein the total emulsifier content is less than 30% by weight of the total composition.

In one embodiment, there is provided a self-emulsifying pharmaceutical composition comprising:
a. an unsaturated free fatty acid;
b. a polyol ester (such as a polyol stearate, for example PEG100 stearate) emulsifier having a HLB value greater than about 15;
c. a second emulsifier having a HLB value less than about 5; and
d. lopinavir;
   wherein the total emulsifier content is less than 20% by weight of the total composition.

In one embodiment, there is provided a self-emulsifying pharmaceutical composition comprising:
a. an unsaturated free fatty acid;
b. a polysorbate emulsifier having a HLB value greater than about 15 (such as polysorbate 20);
c.
d. a second emulsifier having a HLB value less than about 5; and
e. lopinavir and ritonavir;
   wherein the total emulsifier content is less than 30% by weight of the total composition.

In one embodiment, there is provided a self-emulsifying pharmaceutical composition comprising:
a. an unsaturated free fatty acid;
b. a first emulsifier having a HLB value greater than about 15;
c. a monoglyceride (such as glycerol monooleate) emulsifier having a HLB value less than about 5; and
d. lopinavir;
   wherein the total emulsifier content is less than 30% by weight of the total composition.

In one embodiment, there is provided a self-emulsifying pharmaceutical composition comprising:
a. an unsaturated free fatty acid;
b. a first emulsifier having a HLB value greater than about 15;
c. a monoglyceride (such as glycerol monooleate) emulsifier having a HLB value less than about 5; and
d. lopinavir;
   wherein the total emulsifier content is less than 20% by weight of the total composition.

In one embodiment, there is provided a self-emulsifying pharmaceutical composition comprising:
a. an unsaturated free fatty acid;
b. a first emulsifier having a HLB value greater than about 15;
c. a monoglyceride (such as glycerol monooleate) emulsifier having a HLB value less than about 5;
d. a third emulsifier; and
e. lopinavir;
   wherein the total emulsifier content is less than 30% by weight of the total composition.

In one embodiment, there is provided a self-emulsifying pharmaceutical composition comprising:
a. an unsaturated free fatty acid;
b. a first emulsifier having a HLB value greater than about 15;
c. a monoglyceride (such as glycerol monooleate) emulsifier having a HLB value less than about 5;
d. a third emulsifier having a HLB value between about 8 and about 15 (such as between about 10 and about 15, or between about 12 and about 14); and
e. lopinavir;
   wherein the total emulsifier content is less than 30% by weight of the total composition.

In one embodiment, there is provided a self-emulsifying pharmaceutical composition comprising:
a. oleic acid;
b. a polyol ester (such as a polyol stearate, for example PEG100 stearate) emulsifier having a HLB value greater than about 15;
c. a second emulsifier having a HLB value less than about 5; and
d. lopinavir;
   wherein the total emulsifier content is less than 30% by weight of the total composition.

In one embodiment, there is provided a self-emulsifying pharmaceutical composition comprising:
a. oleic acid;
b. a polyol ester (such as a polyol stearate, for example PEG100 stearate) emulsifier having a HLB value greater than about 15;
c. a second emulsifier having a HLB value less than about 5; and
d. lopinavir;
   wherein the total emulsifier content is less than 20% by weight of the total composition.

In one embodiment, there is provided a self-emulsifying pharmaceutical composition comprising:
a. oleic acid;
b. a polyol ester (such as a polyol stearate, for example PEG100 stearate) emulsifier having a HLB value greater than about 15;
c. a second emulsifier having a HLB value less than about 5; and
d. lopinavir and ritonavir;
   wherein the total emulsifier content is less than 30% by weight of the total composition.

In one embodiment, there is provided a self-emulsifying pharmaceutical composition comprising:
a. oleic acid;
b. a first emulsifier having a HLB value greater than about 15;
c. a monoglyceride (such as glycerol monooleate) emulsifier having a HLB value less than about 5; and
d. lopinavir;
   wherein the total emulsifier content is less than 30% by weight of the total composition.

In one embodiment, there is provided a self-emulsifying pharmaceutical composition comprising:
a. oleic acid;
b. a first emulsifier having a HLB value greater than about 15;
c. a monoglyceride (such as glycerol monooleate) emulsifier having a HLB value less than about 5; and
d. lopinavir;
   wherein the total emulsifier content is less than 20% by weight of the total composition.

In one embodiment, there is provided a self-emulsifying pharmaceutical composition comprising:
a. oleic acid;
b. a first emulsifier having a HLB value greater than about 15;
c. a monoglyceride (such as glycerol monooleate) emulsifier having a HLB value less than about 5; and
d. lopinavir and ritonavir;
   wherein the total emulsifier content is less than 30% by weight of the total composition.

In one embodiment, there is provided a self-emulsifying pharmaceutical composition comprising:
a. oleic acid;
b. a polyol ester (such as a polyol stearate, for example PEG100 stearate) emulsifier having a HLB value greater than about 15;
c. a monoglyceride (such as glycerol monooleate) emulsifier having a HLB value less than about 5; and
d. lopinavir;
   wherein the total emulsifier content is less than 30% by weight of the total composition.

In one embodiment, there is provided a self-emulsifying pharmaceutical composition comprising:
a. oleic acid;
b. a polyol ester (such as a polyol stearate, for example PEG100 stearate) emulsifier having a HLB value greater than about 15;
c. a monoglyceride (such as glycerol monooleate) emulsifier having a HLB value less than about 5; and
d. lopinavir;
   wherein the total emulsifier content is less than 20% by weight of the total composition.

In one embodiment, there is provided a self-emulsifying pharmaceutical composition comprising:
a. oleic acid;
b. a polyol ester (such as a polyol stearate, for example PEG100 stearate) emulsifier having a HLB value greater than about 15;
c. a monoglyceride (such as glycerol monooleate) emulsifier having a HLB value less than about 5; and
d. lopinavir and ritonavir;
   wherein the total emulsifier content is less than 30% by weight of the total composition.

In one embodiment, there is provided a self-emulsifying pharmaceutical composition comprising:
a. oleic acid;
b. PEG100 stearate;
c. glycerol monooleate; and
d. lopinavir;
   wherein the total emulsifier content is less than 30% (such as less than 15%) by weight of the total composition.

In one embodiment, there is provided a self-emulsifying pharmaceutical composition comprising:
a. oleic acid;
b. a first emulsifier having a HLB value greater than about 15;
c. a monoglyceride (such as glycerol monooleate) emulsifier having a HLB value less than about 5;
d. a third emulsifier having a HLB value between about 8 and about 15 (such as between about 10 and about 15, or between about 12 and about 14); and
e. lopinavir and ritonavir;
   wherein the total emulsifier content is less than 30% by weight of the total composition.

In one embodiment, there is provided a self-emulsifying pharmaceutical composition comprising:
a. oleic acid;
b. a polysorbate emulsifier having a HLB value greater than about 15 (such as polysorbate 20);
c. a monoglyceride (such as glycerol monooleate) emulsifier having a HLB value less than about 5;
d. a third emulsifier having a HLB value between about 8 and about 15 (such as between about 10 and about 15, or between about 12 and about 14); and
e. lopinavir and ritonavir;
   wherein the total emulsifier content is less than 30% by weight of the total composition.

In one embodiment, there is provided a self-emulsifying pharmaceutical composition comprising:
a. oleic acid;
b. a polysorbate emulsifier having a HLB value greater than about 15 (such as polysorbate 20);
c. a monoglyceride (such as glycerol monooleate) emulsifier having a HLB value less than about 5;
d. a polyoxyl castor oil derivative (such as PEG 35 castor oil) emulsifier having a HLB value between about 10 and about 15; and
e. lopinavir and ritonavir;
   wherein the total emulsifier content is less than 30% by weight of the total composition

In one embodiment, there is provided a self-emulsifying pharmaceutical composition comprising:
a. oleic acid;
b. PEG100 stearate;
c. glycerol monooleate; and
d. lopinavir;
   wherein the total emulsifier content is less than 30% (such as less than 15%) by weight of the total composition.

In one embodiment, there is provided a self-emulsifying pharmaceutical composition comprising:
a. oleic acid;
b. PEG100 stearate;
c. glycerol monooleate; and
d. lopinavir and ritonavir;
   wherein the total emulsifier content is less than 30% (such as less than 15%) by weight of the total composition.

In one embodiment, there is provided a self-emulsifying pharmaceutical composition comprising:
a. oleic acid;
b. polysorbate 20;
c. glycerol monooleate; and
d. lopinavir;
   wherein the total emulsifier content is less than 30% (such as less than 15%) by weight of the total composition.

In one embodiment, there is provided a self-emulsifying pharmaceutical composition comprising:
a. oleic acid;
b. polysorbate 20;
c. glycerol monooleate; and
d. lopinavir and ritonavir;
   wherein the total emulsifier content is less than 30% (such as less than 15%) by weight of the total composition.

In one embodiment, there is provided a self-emulsifying pharmaceutical composition comprising:
a. oleic acid;
b. polysorbate 20;
c. glycerol monooleate;
d. PEG 35 castor oil; and
e. lopinavir;
   wherein the total emulsifier content is less than 30% (such as less than 15%) by weight of the total composition.

In one embodiment, there is provided a self-emulsifying pharmaceutical composition comprising:
a. oleic acid;
b. polysorbate 20;
c. glycerol monooleate;
d. PEG 35 castor oil; and
e. lopinavir and ritonavir;
   wherein the total emulsifier content is less than 30% (such as less than 15%) by weight of the total composition.

In one embodiment, there is provided a self-emulsifying pharmaceutical composition comprising:
a. about 50 to about 80% by weight (such as about 65 to about 75% by weight) of an unsaturated free fatty acid;
b. about 3% to about 6% by weight of a first emulsifier having a HLB value greater than about 14;
c. about 4% to about 7% by weight of a second emulsifier having a HLB value less than about 6; and
d. about 10% to about 20% by weight of lopinavir;
   wherein the total emulsifier content is less than 30% by weight of the total composition.

In one embodiment, there is provided a self-emulsifying pharmaceutical composition comprising:
a. about 65 to about 75% by weight of an unsaturated free fatty acid;
b. about 3% to about 5% by weight of a first emulsifier having a HLB value greater than about 14;
c. about 5% to about 7% by weight of a second emulsifier having a HLB value less than about 6; and
d. about 15% to about 20% by weight of lopinavir;
   wherein the total emulsifier content is less than 30% (such as less than 15%) by weight of the total composition.

In one embodiment, there is provided a self-emulsifying pharmaceutical composition comprising:
a. about 65 to about 75% by weight of an unsaturated free fatty acid;
b. about 3% to about 6% by weight of a first emulsifier having a HLB value greater than about 14;
c. about 4% to about 7% by weight of a second emulsifier having a HLB value less than about 6; and
d. lopinavir and ritonavir;
   wherein the total emulsifier content is less than 30% (such as less than 15%) by weight of the total composition.

In one embodiment, there is provided a self-emulsifying pharmaceutical composition comprising:
a. about 65 to about 75% by weight of oleic acid;
b. about 3% to about 6% by weight of a first emulsifier having a HLB value greater than about 14;
c. about 4% to about 7% by weight glycerol monooleate; and
d. lopinavir;
   wherein the total emulsifier content is less than 15% by weight of the total composition.

In one embodiment, there is provided a self-emulsifying pharmaceutical composition comprising:
a. about 65 to about 75% by weight of oleic acid;
b. about 3% to about 6% by weight of PEG100 stearate or polysorbate 20;
c. about 4% to about 7% by weight glycerol monooleate; and
d. lopinavir and optionally ritonavir;
   wherein the total emulsifier content is less than 15% by weight of the total composition.

In one embodiment, there is provided a self-emulsifying pharmaceutical composition comprising:
a. about 65 to about 75% by weight of oleic acid;
b. about 3% to about 6% by weight of PEG100 stearate or polysorbate 20;
c. about 4% to about 7% by weight glycerol monooleate; and
d. lopinavir and ritonavir;
   wherein the total emulsifier content is less than 15% by weight of the total composition and wherein the wt/wt ratio of lopinavir to ritonavir present in the composition is between about 11:1 to about 13:1 (such as about 12:1).

In one embodiment, there is provided a self-emulsifying pharmaceutical composition comprising:
a. about 68 to about 72% by weight of oleic acid;
b. about 3% to about 6% by weight of PEG100 stearate;
c. about 4% to about 7% by weight glycerol monooleate;
d. about 15% to about 20% by weight lopinavir; and
e. about 1.3% to about 1.7% by weight ritonavir;
   wherein the total emulsifier content is about 10% by weight of the total composition and wherein the wt/wt ratio of lopinavir to ritonavir present in the composition is about 12:1.

In one embodiment, there is provided a self-emulsifying pharmaceutical composition comprising:
a. about 60% to about 75% by weight of oleic acid;
b. about 3% to about 6% by weight of a first emulsifier having a HLB value greater than about 14;
c. about 4% to about 7% by weight glycerol monooleate;
d. about 4% to about 6% by weight of a third emulsifier having a HLB value between about 8 and about 15; and
e. lopinavir;
   wherein the total emulsifier content is less than 30% by weight of the total composition.

In one embodiment, there is provided a self-emulsifying pharmaceutical composition comprising:
a. about 60% to about 75% by weight of oleic acid;
b. about 3% to about 6% by weight of PEG100 stearate or polysorbate 20;
c. about 4% to about 7% by weight glycerol monooleate;
d. about 4% to about 6% by weight of a third emulsifier having a HLB value between about 8 and about 15; and
e. lopinavir and optionally ritonavir;
   wherein the total emulsifier content is less than 30% by weight of the total composition.

In one embodiment, there is provided a self-emulsifying pharmaceutical composition comprising:
a. about 60 to about 75% by weight of oleic acid;
b. about 3% to about 6% by weight of PEG100 stearate or polysorbate 20;
c. about 4% to about 7% by weight glycerol monooleate;
d. about 4% to about 6% by weight PEG 35 castor oil; and
e. lopinavir and ritonavir;
   wherein the total emulsifier content is less than 30% by weight of the total composition and wherein the wt/wt ratio of lopinavir to ritonavir present in the composition is between about 11:1 to about 13:1 (such as about 12:1).

In one embodiment, there is provided a self-emulsifying pharmaceutical composition comprising:
a. about 60 to about 72% by weight of oleic acid;
b. about 3% to about 6% by weight of PEG100 stearate or polysorbate 20;
c. about 4% to about 7% by weight glycerol monooleate;
d. about 4% to about 6% by weight PEG 35 castor oil;
e. about 15% to about 20% by weight lopinavir; and
f. about 1.3% to about 1.7% by weight ritonavir;
   wherein the total emulsifier content is less than 30% by weight of the total composition and wherein the wt/wt ratio of lopinavir to ritonavir present in the composition is about 12:1.

In one embodiment, there is provided a self-emulsifying pharmaceutical composition comprising:
a. about 60 to about 65% by weight of oleic acid;
b. about 3% to about 5% by weight of polysorbate 20;
c. about 4% to about 6% by weight glycerol monooleate;
d. about 4% to about 6% by weight PEG 35 castor oil;
e. about 17% to about 23% by weight lopinavir; and
f. about 1.5% to about 1.9% by weight ritonavir;
   wherein the total emulsifier content is less than 30% by weight of the total composition and wherein the wt/wt ratio of lopinavir to ritonavir present in the composition is about 12:1.

In one embodiment, there is provided a self-emulsifying pharmaceutical composition comprising:
a. about 50 to about 55% by weight of oleic acid;
b. about 15% to about 20% by weight of polysorbate 20;
c. about 4% to about 7% by weight glycerol monooleate;
d. about 4% to about 6% by weight PEG 35 castor oil;
e. about 15% to about 19% by weight lopinavir; and
f. about 1.2% to about 1.5% by weight ritonavir;
   wherein the total emulsifier content is less than 30% by weight of the total composition and wherein the wt/wt ratio of lopinavir to ritonavir present in the composition is about 12:1.

In one embodiment, there is provided a self-emulsifying pharmaceutical composition comprising:
a. about 45 to about 55% by weight of oleic acid;
b. about 10% to about 14% by weight of polysorbate 20;
c. about 12% to about 16% by weight glycerol monooleate;
d. about 3% to about 6% by weight PEG 35 castor oil;
e. about 15% to about 23% by weight lopinavir; and
f. about 1.2% to about 1.9% by weight ritonavir;
   wherein the total emulsifier content is less than 30% by weight of the total composition and wherein the wt/wt ratio of lopinavir to ritonavir present in the composition is about 12:1.

In an embodiment, there is provided a self-emulsifying pharmaceutical composition consisting of:
a. an unsaturated free fatty acid;
b. a first emulsifier having a HLB value greater than about 14;
c. a second emulsifier having a HLB value less than about 6;
d. an antioxidant;
e. lopinavir; and
f. ritonavir;
   wherein the total emulsifier content is less than 30% by weight of the total composition.

In an embodiment, there is provided a self-emulsifying pharmaceutical composition consisting of:
a. oleic acid;
b. a first emulsifier having a HLB value greater than about 14;
c. a second emulsifier having a HLB value less than about 6;
d. an antioxidant;
e. lopinavir; and
f. ritonavir;
   wherein the total emulsifier content is less than 30% by weight of the total composition.

In an embodiment, there is provided a self-emulsifying pharmaceutical composition consisting of:
a. an unsaturated free fatty acid;
b. a polyol ester emulsifier having a HLB value greater than about 14 (such as PEG100 stearate);
c. a second emulsifier having a HLB value less than about 6;
d. an antioxidant;
e. lopinavir; and
f. ritonavir;
   wherein the total emulsifier content is less than 30% by weight of the total composition.

In an embodiment, there is provided a self-emulsifying pharmaceutical composition consisting of:
a. an unsaturated free fatty acid;
b. a first emulsifier having a HLB value greater than about 14;
c. a monoglyceride emulsifier having a HLB value less than about 6 (such as glycerol monooleate);
d. an antioxidant;
e. lopinavir; and
f. ritonavir;
   wherein the total emulsifier content is less than 30% by weight of the total composition.

In an embodiment, there is provided a self-emulsifying pharmaceutical composition consisting of:
a. an unsaturated free fatty acid;
b. a polysorbate emulsifier having a HLB value greater than about 14 (such as polysorbate 20);
c. a monoglyceride emulsifier having a HLB value less than about 6 (such as glycerol monooleate);
d. an antioxidant;
e. lopinavir; and
f. ritonavir;
   wherein the total emulsifier content is less than 30% by weight of the total composition.

In an embodiment, there is provided a self-emulsifying pharmaceutical composition consisting of:
a. oleic acid;
b. a first emulsifier having a HLB value greater than 14;
c. a monoglyceride emulsifier having a HLB value less than about 6 (such as glycerol monooleate);
d. an antioxidant;
e. lopinavir; and
f. ritonavir;
   wherein the total emulsifier content is less than 20% by weight of the total composition.

In an embodiment, there is provided a self-emulsifying pharmaceutical composition consisting of:
a. oleic acid;
b. a first emulsifier having a HLB value greater than 14;
c. a monoglyceride emulsifier having a HLB value less than about 6 (such as glycerol monooleate);
d. an antioxidant;
e. lopinavir; and
f. ritonavir;
   wherein the total emulsifier content is about 8% to about 12% by weight of the total composition and the wt/wt ratio of monoglyceride emulsifier to first emulsifier present in the composition is between about 1.1:1 and 1.6:1 (such as between about 1.4:1 and 1.6:1).

In an embodiment, there is provided a self-emulsifying pharmaceutical composition consisting of:
a. oleic acid;
b. a polyol ester emulsifier having a HLB value greater than about 14 (such as PEG100 stearate or polysorbate 20);
c. a monoglyceride emulsifier having a HLB value less than about 6 (such as glycerol monooleate);
d. an antioxidant;
e. lopinavir; and
f. ritonavir;
   wherein the total emulsifier content is about 8% to about 12% by weight of the total composition and wherein the wt/wt ratio of lopinavir to ritonavir present in the composition is about 12:1.

In an embodiment, there is provided a self-emulsifying pharmaceutical composition consisting of:
a. an unsaturated free fatty acid;
b. a first emulsifier having a HLB value greater than about 14;
c. a second emulsifier having a HLB value less than about 6;
d. a third emulsifier;
e. an antioxidant;
f. lopinavir; and
g. ritonavir;
   wherein the total emulsifier content is less than 30% by weight of the total composition.

In an embodiment, there is provided a self-emulsifying pharmaceutical composition consisting of:
a. oleic acid;
b. a first emulsifier having a HLB value greater than about 14;
c. a second emulsifier having a HLB value less than about 6;
d. a third emulsifier;
e. an antioxidant;
f. lopinavir; and
g. ritonavir;
   wherein the total emulsifier content is less than 30% by weight of the total composition.

In an embodiment, there is provided a self-emulsifying pharmaceutical composition consisting of:
a. an unsaturated free fatty acid (such as oleic acid);
b. a first emulsifier having a HLB value greater than about 14;
c. a second emulsifier having a HLB value less than about 6;
d. a third emulsifier having a HLB value between about 8 and about 15;
e. an antioxidant;
f. lopinavir; and
g. ritonavir;
   wherein the total emulsifier content is less than 30% by weight of the total composition.

In an embodiment, there is provided a self-emulsifying pharmaceutical composition consisting of:
a. an unsaturated free fatty acid (such as oleic acid);
b. a first emulsifier having a HLB value greater than about 14;
c. a second emulsifier having a HLB value less than about 6;
d. a third emulsifier having a HLB value between about 10 and about 15 (such as between about 12 and about 14);
e. an antioxidant;
f. lopinavir; and
g. ritonavir;
   wherein the total emulsifier content is less than 30% by weight of the total composition.

In an embodiment, there is provided a self-emulsifying pharmaceutical composition consisting of:
a. an unsaturated free fatty acid;
b. a first emulsifier having a HLB value greater than about 14;
c. a monoglyceride emulsifier having a HLB value less than about 6 (such as glycerol monooleate);
d. a third emulsifier having a HLB value between about 10 and about 15 (such as between about 12 and about 14);
e. an antioxidant;
f. lopinavir; and
g. ritonavir;
   wherein the total emulsifier content is less than 30% by weight of the total composition.

In an embodiment, there is provided a self-emulsifying pharmaceutical composition consisting of:
a. an unsaturated free fatty acid;
b. a polysorbate emulsifier having a HLB value greater than about 14 (such as polysorbate 20);
c. a monoglyceride emulsifier having a HLB value less than about 6 (such as glycerol monooleate);
d. a third emulsifier having a HLB value between about 10 and about 15 (such as between about 12 and about 14);
e. an antioxidant;
f. lopinavir; and
g. ritonavir;
   wherein the total emulsifier content is less than 30% by weight of the total composition.

In an embodiment, there is provided a self-emulsifying pharmaceutical composition consisting of:
a. an unsaturated free fatty acid;
b. a polysorbate emulsifier having a HLB value greater than about 14 (such as polysorbate 20);
c. a monoglyceride emulsifier having a HLB value less than about 6 (such as glycerol monooleate);
d. a polyoxyl castor oil derivative emulsifier (such as PEG 35 castor oil);
e. an antioxidant;
f. lopinavir; and
g. ritonavir;
   wherein the total emulsifier content is less than 30% by weight of the total composition.

In an embodiment, there is provided a self-emulsifying pharmaceutical composition consisting of:
a. oleic acid;
b. a first emulsifier having a HLB value greater than 14;
c. a monoglyceride emulsifier having a HLB value less than about 6 (such as glycerol monooleate);
d. a polyoxyl castor oil derivative emulsifier (such as PEG 35 castor oil);
e. an antioxidant;
f. lopinavir; and
g. ritonavir;
   wherein the total emulsifier content is less than 20% by weight of the total composition.

In an embodiment, there is provided a self-emulsifying pharmaceutical composition consisting of:
a. an unsaturated free fatty acid (such as oleic acid);
b. a first emulsifier having a HLB value greater than about 14;
c. a second emulsifier having a HLB value less than about 6;
d. a third emulsifier having a HLB value between about 8 and about 15;
e. an antioxidant;
f. lopinavir; and
g. ritonavir;
   wherein the total emulsifier content is less than 30% by weight of the total composition and the wt/wt ratio of lopinavir to ritonavir present in the composition is about 12:1.

In an embodiment, there is provided a self-emulsifying pharmaceutical composition consisting of:
a. an unsaturated free fatty acid (such as oleic acid);
b. a first emulsifier having a HLB value greater than about 14;
c. a second emulsifier having a HLB value less than about 6;
d. a third emulsifier having a HLB value between about 8 and about 15;
e. an antioxidant;
f. lopinavir; and
g. ritonavir;
   wherein the total emulsifier content is less than 30% by weight of the total composition, the wt/wt ratio of lopinavir to ritonavir present in the composition is about 12:1 and the wt/wt ratio of second emulsifier to first emulsifier present in the composition is between about 1.1:1 and 1.6:1 (such as between about 1.4:1 and 1.6:1).

In an embodiment, there is provided a self-emulsifying pharmaceutical composition consisting of:
a. oleic acid;
b. polysorbate 20;
c. glycerol monooleate;
d. PEG 35 castor oil;
e. an antioxidant (such as butylated hydroxytoluene);
f. lopinavir; and
g. ritonavir;
   wherein the total emulsifier content is less than 30% by weight of the total composition and the wt/wt ratio of lopinavir to ritonavir present in the composition is about 12:1.

In an embodiment, there is provided a self-emulsifying pharmaceutical composition consisting of:
a. oleic acid;
b. polysorbate 20;
c. glycerol monooleate;
d. PEG 35 castor oil;
e. an antioxidant (such as butylated hydroxytoluene);
f. lopinavir; and
g. ritonavir;
   wherein the total emulsifier content is less than 30% by weight of the total composition, the wt/wt ratio of lopinavir to ritonavir present in the composition is about 12:1 and the wt/wt ratio of glycerol monooleate to polysorbate 20 present in the composition is between about 1.1:1 and 1.6:1 (such as between about 1.4:1 and 1.6:1).

### Administration

The pharmaceutical compositions according to the present invention may be administered by any suitable means, as may be determined by a skilled person on the basis of the active pharmaceutical ingredient(s) and the disease or disorder being treated with the composition. Preferably, the pharmaceutical composition is for oral or topical administration.

Typically the pharmaceutical compositions according to the present invention are liquid compositions. Therefore, for ease of dosing and administration, the compositions may be filled into a capsule.

In one aspect, there is provided a capsule comprising a pharmaceutical composition of the first aspect of the invention. In one embodiment, the capsule is for intra-vaginal administration of the pharmaceutical composition. In another embodiment, the capsule is for oral administration of the pharmaceutical composition.

In an embodiment, the capsule is a gelatin capsule. In one embodiment, the capsule is a hard capsule. In another embodiment, the capsule is a soft capsule. In one embodiment, the capsule is a hard gelatin capsule. In another preferred embodiment, the capsule is a soft gelatin capsule.

### Self-Emulsifying Drug Delivery Performance

The pharmaceutical compositions of the present invention comprise at least one active pharmaceutical ingredient (lopinavir) dissolved or suspended in an unsaturated fatty acid. When introduced into aqueous-based environments, the compositions rapidly emulsify into stable emulsions as a result of the at least two emulsifiers also present in the compositions. The droplet size of the resultant emulsion can be important for stabilising the emulsion and it can also influence the rate of API dissolution.

Emulsion droplet size may be determined by dispersion of a sample of the composition in water and analysing the resulting emulsion by dynamic light scattering using a Zetasizer instrument. Details of a suitable method for determining the average emulsion droplet size via dynamic light scattering are given in Example 8 below.

In an embodiment, a pharmaceutical composition as defined herein has an average emulsion droplet size of less than 1500 nm, such as less than 1200 nm, less than 1000 nm, less than 750 nm, less than 600 nm, less than 500 nm, less than 400 nm, or less than 300 nm. In a preferred embodiment, a pharmaceutical composition as defined herein has an average emulsion droplet size of less than 750 nm. In an embodiment, a pharmaceutical composition as defined herein has an average emulsion droplet size of 100 to 1500 nm, such as 100 to 1000 nm, 150 to 750 nm, 200 to 750 nm, or 200 to 500 nm.

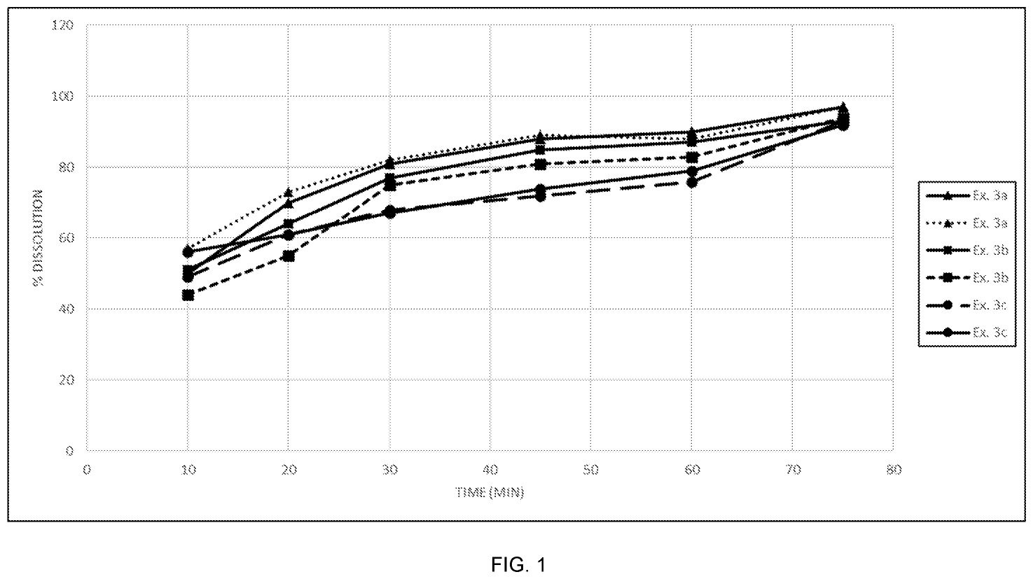

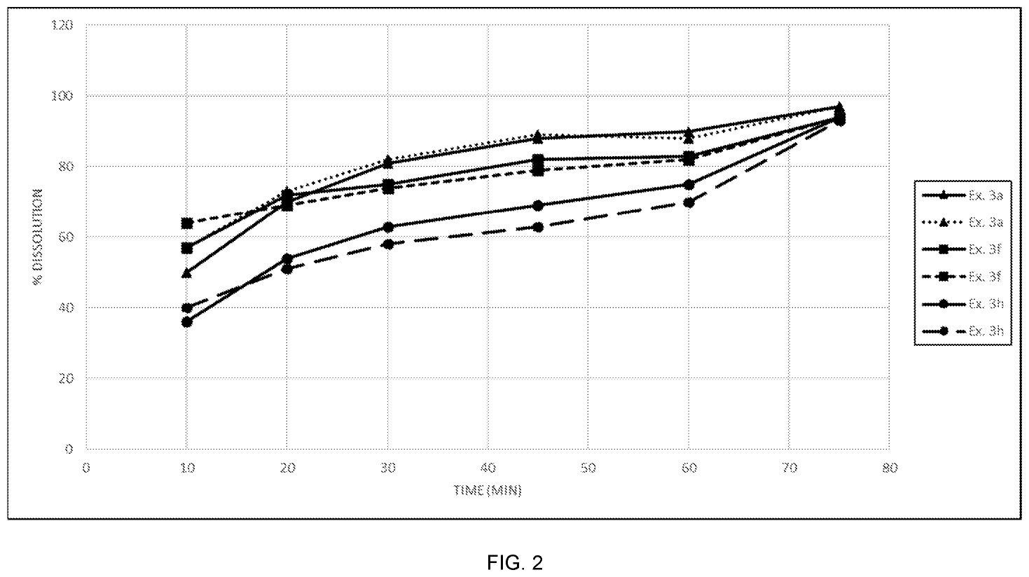

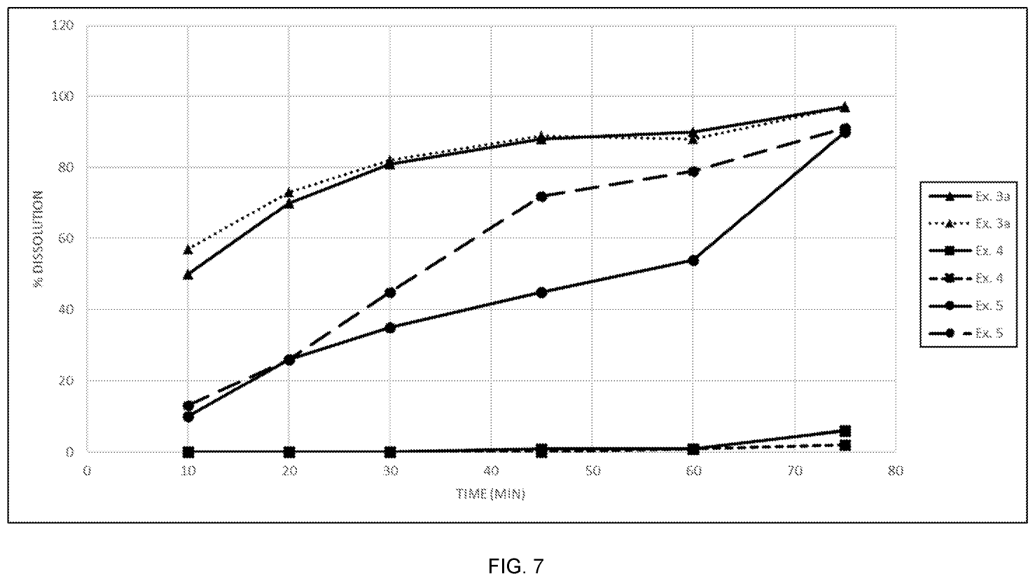

Pharmaceutical compositions according to the present invention have been found to surprisingly demonstrate superior in vitro API dissolution, despite having low total emulsifier contents (see FIG. 1). It has been found also that when the in vitro API dissolution was compared to equivalent formulations containing only a single emulsifier (either PEG100 stearate only or glycerol monooleate only), then the single emulsifier formulations exhibited slower API dissolution, even when the total emulsifier content was the same as the two emulsifier formulations (see FIGS. 2-6). Alternative lopinavir and ritonavir formulations with low total emulsifier contents (comparative formulations 1 & 2—Examples 4 & 5) were found to have significantly slower in vitro API dissolution profiles than pharmaceutical compositions according to the present invention containing lopinavir and ritonavir (see FIG. 7). In vitro dissolution behaviour provides a good indication of how rapidly the active pharmaceutical ingredient(s) becomes available for systemic absorption and therefore the pharmaceutical compositions of the present invention are predicted to provide good API bioavailability.

In vitro dissolution testing may be carried out by various dissolution testing models which will be apparent to one of skill in the art. In the following embodiments the dissolution profiles are those obtained by filling the composition in hard gel capsules with sinkers and measuring the API dissolution in 0.7% w/v SLS media at about 37° C. using USP apparatus II at 25 rpm for 0-60 minutes and 200 rpm for 60-75 minutes. At each time point, the media is sampled and analysed for API content by HPLC and the % dissolution is determined.

In an embodiment, the pharmaceutical composition has a dissolution profile which comprises greater than 20% lopinavir dissolution after 10 minutes, such as greater than 30% lopinavir dissolution, greater than 40% lopinavir dissolution, or greater than 50% lopinavir dissolution after 10 minutes, when measuring the dissolution in 0.7% w/v SLS media at about 37° C. using USP apparatus 11 at 25 rpm for 0-60 minutes and 200 rpm for 60-75 minutes.

In a preferred embodiment, the pharmaceutical composition has a dissolution profile which comprises greater than 60% lopinavir dissolution after 45 minutes, such as greater than 65% lopinavir dissolution, greater than 70% lopinavir dissolution, greater than 75% lopinavir dissolution, or greater than 80% lopinavir dissolution after 45 minutes, when measuring the dissolution in 0.7% w/v SLS media at about 37° C. using USP apparatus II at 25 rpm for 0-60 minutes and 200 rpm for 60-75 minutes.

In a preferred embodiment, the pharmaceutical composition has a dissolution profile which comprises greater than 90% lopinavir dissolution after 75 minutes, such as greater than 92% lopinavir dissolution, greater than 95% lopinavir dissolution, or greater than 97% lopinavir dissolution after 75 minutes, when measuring the dissolution in 0.7% w/v SLS media at about 37° C. using USP apparatus II at 25 rpm for 0-60 minutes and 200 rpm for 60-75 minutes.

In an embodiment, the pharmaceutical composition has a dissolution profile which comprises greater than 20% lopinavir dissolution after 10 minutes, and greater than 60% lopinavir dissolution after 45 minutes, when measuring the dissolution in 0.7% w/v SLS media at about 37° C. using USP apparatus II at 25 rpm for 0-60 minutes and 200 rpm for 60-75 minutes. In an embodiment, the pharmaceutical composition has a dissolution profile which comprises greater than 40% lopinavir dissolution after 10 minutes, and greater than 70% lopinavir dissolution after 45 minutes, when measuring the dissolution in 0.7% w/v SLS media at about 37° C. using USP apparatus II at 25 rpm for 0-60 minutes and 200 rpm for 60-75 minutes.

In an embodiment, the pharmaceutical composition has a dissolution profile which comprises greater than 20% lopinavir dissolution after 10 minutes, greater than 60% lopinavir dissolution after 45 minutes and greater than 90% lopinavir dissolution after 75 minutes, when measuring the dissolution in 0.7% w/v SLS media at about 37° C. using USP apparatus II at 25 rpm for 0-60 minutes and 200 rpm for 60-75 minutes. In an embodiment, the pharmaceutical composition has a dissolution profile which comprises greater than 40% lopinavir dissolution after 10 minutes, greater than 70% lopinavir dissolution after 45 minutes and greater than 90% lopinavir dissolution after 75 minutes, when measuring the dissolution in 0.7% w/v SLS media at about 37° C. using USP apparatus II at 25 rpm for 0-60 minutes and 200 rpm for 60-75 minutes.

In the following embodiments the dissolution profiles are those obtained by filling the composition in hard gel capsules with sinkers and measuring the API dissolution in 0.7% cetyltrimethylammonium bromide (CTAB) media at 37±0.5° C., carried out in USP II apparatus at 50 rpm for 0 to 60 min. At each time point, the media is sampled and analysed for API content by HPLC and the % dissolution is determined.

In an embodiment, the pharmaceutical composition has a dissolution profile which comprises greater than 30% lopinavir dissolution after 10 minutes, greater than 60% lopinavir dissolution after 30 minutes and greater than 70% lopinavir dissolution after 60 minutes, when measuring the dissolution in 0.7% w/v cetyltrimethylammonium bromide (CTAB) media at 37 f 0.5° C., carded out in USP II apparatus at 50 rpm for 0 to 60 min. In an embodiment, the pharmaceutical composition has a dissolution profile which comprises greater than 50% lopinavir dissolution after 10 minutes, greater than 70% lopinavir dissolution after 30 minutes and greater than 80% lopinavir dissolution after 60 minutes, when measuring the dissolution in 0.7% w/v cetyltrimethylammonium bromide (CTAB) media at 37±0.5° C., carried out in USP II apparatus at 50 rpm for 0 to 60 min.

In the following embodiments the dissolution profiles are those obtained by filling the composition in hard gel capsules with sinkers and measuring the API dissolution in 0.05 M PEG oleyl ether (Brij® 10) with 10 mM sodium monobasic phosphate pH 6.8 media at 37±0.5° C., carried out in USP II apparatus at 50 rpm for 0 to 60 min. At each time point, the media is sampled and analysed for API content by HPLC and the % dissolution is determined.

In an embodiment, the pharmaceutical composition has a dissolution profile which comprises greater than 30% lopinavir dissolution after 10 minutes, greater than 60% lopinavir dissolution after 30 minutes and greater than 80% lopinavir dissolution after 60 minutes, when measuring the dissolution in 0.05 M PEG 10 oleyl ether (Brij® 10) with 10 mM sodium monobasic phosphate pH 6.8 media at 37±0.5° C., carried out in USP II apparatus at 50 rpm for 0 to 60 min. In an embodiment, the pharmaceutical composition has a dissolution profile which comprises greater than 60% lopinavir dissolution after 10 minutes, greater than 80% lopinavir dissolution after 30 minutes and greater than 90% lopinavir dissolution after 60 minutes, when measuring the dissolution in 0.05 M PEG 10 oleyl ether (Brij® 10) with 10 mM sodium monobasic phosphate pH 6.8 media at 37±0.5° C., carried out in USP II apparatus at 50 rpm for 0 to 60 min.

Advantageously, the pharmaceutical compositions of the present invention may typically have good lopinavir dissolution across a range of dissolution media indicative of their potential for providing good lopinavir bioavailability. In an embodiment, the pharmaceutical composition has a dissolution profile which comprises:
(A) greater than 20% lopinavir dissolution after 10 minutes, greater than 60% lopinavir dissolution after 45 minutes and greater than 90% lopinavir dissolution after 75 minutes, when measuring the dissolution in 0.7% w/v SLS media at about 37° C. using USP apparatus II at 25 rpm for 0-60 minutes and 200 rpm for 60-75 minutes;
(B) greater than 30% lopinavir dissolution after 10 minutes, greater than 60% lopinavir dissolution after 30 minutes and greater than 70% lopinavir dissolution after 60 minutes, when measuring the dissolution in 0.7% w/v cetyltrimethylammonium bromide (CTAB) media at 37±0.5° C., carried out in USP II apparatus at 50 rpm for 0 to 60 min; and
(C) greater than 30% lopinavir dissolution after 10 minutes, greater than 60% lopinavir dissolution after 30 minutes and greater than 80% lopinavir dissolution after 60 minutes, when measuring the dissolution in 0.05 M PEG 10 oleyl ether (Brij® 10) with 10 mM sodium monobasic phosphate pH 6.8 media at 37±0.5° C., carried out in USP II apparatus at 50 rpm for 0 to 60 min.

In an embodiment, the pharmaceutical composition has a dissolution profile which comprises:
(A) greater than 40% lopinavir dissolution after 10 minutes, greater than 70% lopinavir dissolution after 45 minutes and greater than 90% lopinavir dissolution after 75 minutes, when measuring the dissolution in 0.7% w/v SLS media at about 37° C. using USP apparatus II at 25 rpm for 0-60 minutes and 200 rpm for 60-75 minutes;
(B) greater than 50% lopinavir dissolution after 10 minutes, greater than 70% lopinavir dissolution after 30 minutes and greater than 80% lopinavir dissolution after 60 minutes, when measuring the dissolution in 0.7% w/v cetyltrimethylammonium bromide (CTAB) media at 37±0.5° C., carried out in USP II apparatus at 50 rpm for 0 to 60 min; and
(C) greater than 60% lopinavir dissolution after 10 minutes, greater than 80% lopinavir dissolution after 30 minutes and greater than 90% lopinavir dissolution after 60 minutes, when measuring the dissolution in 0.05 M PEG 10 oleyl ether (Brij® 10) with 10 mM sodium monobasic phosphate pH 6.8 media at 37±0.5° C., carried out in USP II apparatus at 50 rpm for 0 to 60 min.

Cmax is the maximum serum concentration that a drug achieves in a specified compartment or test area of the body after the drug has been administrated. A higher Cmax value corresponds to higher systemic drug levels and therefore greater bioavailability of the drug. A PK study protocol to determine Cmax for a composition according to the present invention is described in Example 9. In an embodiment, a pharmaceutical composition according to the present invention, when dosed daily as an 800 mg or 900 mg capsule containing 150 mg of lopinavir, has a mean Cmax of greater than 500 μg/ml, such as greater than 1 ng/ml, greater than 500 ng/ml, or greater than 1 μg/ml.

### Medical Uses

In one embodiment, there is provided a self-emulsifying pharmaceutical composition as defined herein for use as a medicament.

In one embodiment, the pharmaceutical composition is for use as a medicament wherein the pharmaceutical composition is applied topically (e.g. to a mucosal surface) or administered orally.

In a preferred embodiment, the self-emulsifying pharmaceutical compositions of the present invention are encapsulated in a capsule. Such capsules provide a convenient delivery form for both oral and topical (such as intra-vaginal) administration of the pharmaceutical composition. In one embodiment, there is provided a self-emulsifying pharmaceutical composition as defined herein for use as a medicament, wherein the pharmaceutical composition is administered orally as a capsule. In an embodiment, there is provided a self-emulsifying pharmaceutical composition as defined herein for use as a medicament, wherein the pharmaceutical composition is administered topically (e.g. intra-vaginally) as a capsule.

In one embodiment, the pharmaceutical compositions are useful in the treatment and/or prevention of diseases and/or disorders. In one embodiment, the pharmaceutical compositions are useful in the treatment of benign proliferative disorders.

In one embodiment, the pharmaceutical compositions are useful in the treatment of cancer and particularly useful for preventing the development of cancers. Accordingly, normal subjects (i.e. subjects with no detectable cancer), subjects with pre-malignant cells or particularly cancer prone subjects may be treated by topical administration of compositions according to the invention with a view to preventing the development of cancer.

Disclosed herein are pharmaceutical compositions comprising lopinavir and ritonavir for use as a medicament in the treatment of cancer or benign proliferative disorders (e.g. warts) or in the prevention of the development of cancer. In an embodiment, there is provided a method of treating and/or inhibiting the development or progression of cancers and benign proliferative disorders in a subject in need of such treatment or inhibition comprising administering a therapeutically effective amount of a pharmaceutical composition as disclosed herein.

The invention, to the extent that it is applicable to the prevention and treatment of cancer, may be applied to a wide range of cancers such as ovarian carcinoma, breast carcinoma, lung carcinoma, uterine carcinoma, cervical carcinoma and thyroid carcinoma. The invention is applicable particularly, but by no means exclusively, to pre-cancerous conditions and cancers caused by oncogenic viruses, e.g. high-risk or even low-risk forms of human papilloma viruses (HPVs).

Conveniently, the compositions may be administered to treat, and particularly prevent, the development of cervical cancer. Conveniently, the inhibitors are used to treat, or prevent the development of cervical cancers caused by HPV (particularly high-risk types of HPV such as HPV16 or HPV 18). In an embodiment, there is provided a method of treating and/or inhibiting the development or progression of cancers and benign proliferative disorders in a subject in need of such treatment or inhibition comprising administering a therapeutically effective amount of a pharmaceutical composition as disclosed herein, wherein the cancer or disorder is caused or induced by a human papilloma virus (HPV).

The compositions may be used to prevent or treat cancer as a monotherapy (i.e. including the use of the pharmaceutical composition comprising two or more active pharmaceutical ingredients) or in combination with other compounds or treatments used in cancer therapy (e.g. chemotherapeutic agents, radiotherapy).

Disclosed herein are pharmaceutical compositions comprising lopinavir and ritonavir for use as a medicament in the treatment of cancer or benign proliferative disorders (e.g. warts) or in the prevention of the development of cancer.

Conveniently, the compositions are used to treat humans. However, it will be appreciated that the compositions may also have some veterinary use.

### Dosing

It will be appreciated that the amount of at least one active pharmaceutical ingredient (lopinavir) required is determined by biological activity and bioavailability, which in turn depends, in part, on the precise mode of administration, the physicochemical properties of the pharmaceutical composition employed, and whether the pharmaceutical compositions are being used as a monotherapy or in a combined therapy with other medicines. Indeed, it is also possible that the at least one active pharmaceutical ingredient (lopinavir) could be topically applied in addition to oral dosing of the same at least one active pharmaceutical ingredient or other active pharmaceutical ingredient(s). The frequency of administration will also be influenced by the abovementioned factors and particularly the half-life of the active pharmaceutical ingredients within the subject being treated.

Daily doses may be given as a single administration (e.g. as a soft gel capsule, a hard gel capsule, a pessary or a suppository). Alternatively, administration may be twice or more times during a day. As an example, the pharmaceutical compositions (e.g. as a soft gel capsule or a hard gel capsule) may be topically administered at least once a day, such as once a day, or such as twice a day.

Optimal dosages to be administered may be determined by those skilled in the art, and will vary with the strength of the preparation, the mode of administration, and the advancement of the disease condition. Additional factors depending on the particular subject being treated will result in a need to adjust dosages, including, for example, subject age, weight, gender, diet, and time of administration.

Suitable amounts of the at least one active pharmaceutical ingredient to be given as a daily dose are of about 0.01 mg to about 10 g, such as about 0.1 mg to about 10 g, such as about 1 mg to about 5 g, such as about 1 mg to about 1 g, such as about 5 mg to about 2 g, such as about 10 mg to about 1 g, such as about 5 mg to about 500 mg, such as about 10 mg to about 500 mg, such as about 10 mg to about 400 mg, such as about 5 mg to about 200 mg, such as about 5 mg to about 50 mg, such as about 10 mg to about 40 mg, such as about 20 mg to about 40 mg, such as about 25 mg to about 35 mg, such as about 27 mg to about 32 mg, such as about 29 mg, such as 29 mg, such as about 28.7 mg, such as 28.7 mg, such as about 15 mg to about 35 mg, such as about 20 mg to about 30 mg, such as about 23 mg to about 27 mg, such as about 25 mg, such as 25 mg, such as about 5 mg to about 25 mg, such as about 10 mg to about 20 mg, such as about 12 mg to about 16 mg, such as about 14 mg, such as 14 mg, such as about 14.3 mg, such as about 14.3 mg, such as about 11 mg to about 15 mg, such as about 13 mg, such as 13 mg, such as about 12.5 mg, such as 12.5 mg, such as about 100 mg to about 400 mg, such as about 200 mg to about 400 mg, such as about 250 mg to about 350 mg, such as about 280 mg to about 320 mg, such as about 290 mg to about 310 mg, such as about 300 mg, such as 300 mg, such as about 25 mg to about 325 mg, such as about 50 mg to about 250 mg, such as about 125 mg to about 175 mg, such as about 140 mg to about 160 mg, such as about 150 mg, or such as 150 mg.

Suitable amounts of lopinavir to be given as a daily dose of about 0.01 mg to about 10 g, such as about 0.1 mg to about 10 g, such as about 1 mg to about 5 g, such as about 5 mg to about 2 g, such as about 10 mg to about 1 g, such as about 10 mg to about 500 mg, such as about 10 mg to about 400 mg, such as about 100 mg to about 400 mg, such as about 200 mg to about 400 mg, such as about 250 mg to about 350 mg, such as about 280 mg to about 320 mg, such as about 290 mg to about 310 mg, such as about 300 mg, such as 300 mg, such as about 25 mg to about 325 mg, such as about 50 mg to about 250 mg, such as about 125 mg to about 175 mg, such as about 140 mg to about 160 mg, such as about 150 mg, or such as 150 mg. In a convenient embodiment, the daily dose of lopinavir is about 150 mg or about 300 mg. Most conveniently, the daily dose of lopinavir is 300 mg.

Suitable amounts of ritonavir to be given as a daily dose of about 0.01 mg to about 10 g, such as about 0.1 mg to about 10 g, such as about 1 mg to about 5 g, such as about 1 mg to about 1 g, such as about 5 mg to about 500 mg, such as about 5 mg to about 200 mg, such as about 5 mg to about 50 mg, such as about 10 mg to about 40 mg, such as about 20 mg to about 40 mg, such as about 25 mg to about 35 mg, such as about 27 mg to about 32 mg, such as about 29 mg, such as 29 mg, such as about 28.7 mg, such as 28.7 mg, such as about 15 mg to about 35 mg, such as about 20 mg to about 30 mg, such as about 23 mg to about 27 mg, such as about 25 mg, such as 25 mg, such as about 5 mg to about 25 mg, such as about 10 mg to about 20 mg, such as about 12 mg to about 16 mg, such as about 14 mg, such as 14 mg, such as about 14.3 mg, such as 14.3 mg, such as about 11 mg to about 15 mg, such as about 13 mg, such as 13 mg, such as about 12.5 mg, or such as 12.5 mg. In a convenient embodiment, the daily dose of ritonavir is about 12.5 mg or about 25 mg. Most conveniently, the daily dose of ritonavir is 25 mg.

In one embodiment, about 150 mg of lopinavir and about 12.5 mg ritonavir per day may be administered to a patient in need thereof.

In one embodiment, about 300 mg of lopinavir and about 25 mg ritonavir per day may be administered to a patient in need thereof.

### Treatment Regimens

The pharmaceutical composition may be administered to a subject for as long as treatment is required. The length of time for which treatment will be required will depend upon the exact condition being treated or prevented and its severity. A skilled person will appreciate that treatment should be maintained in view of a number of factors which will include any requirement to eradicate the disease and/or disorder.

In one embodiment, a course of treatment may be for 2-4 weeks, 7-21 days or for about 14 days. After this time a clinician may assess whether the course of treatment has been successful. A decision may then be made whether or not to continue treatment.

It will be appreciated that a clinician may wish to take into account menstruation when deciding on a treatment regimen for women with conditions relating to the cervix. Conveniently, a treatment regimen may be for about 14-21 days and can be administered between menses. A clinician may elect to stop topical treatment of the cervix during menses and recommence a new course of treatment in the next menstrual cycle. By way of example, a treatment regimen can be: (1) 14-21 days of administration; (2) followed by 1-14 days without treatment (during which menses may occur if treating the cervix); and (3) a further cycle of 14 −21 days of treatment if this is considered medically necessary.

### Treatment of HPV Related Dysplasia

Conveniently, the pharmaceutical compositions may be used to treat female subjects having an HPV related dysplasia of the cervix.

As used herein, “dysplasia” encompasses pre-invasive lesions and cancer. HPV related pre-invasive lesions include high grade squamous intraepithelial lesion (HSIL), atypical squamous cells of undetermined significance (ASCUS), and low grade squamous intraepithelial lesion (LSIL). HPV related cancers include, for example, cervical intraepithelial neoplasia (CIN) and invasive cervical cancer (ICC).

The disclosed methods and treatment regimens can be used to treat HPV related dysplasia. In some aspects, for example, the disclosed methods and treatment regimens can be used to treat HSIL. In some aspects, the disclosed methods and treatment regimens can be used to treat ASCUS. In other aspects, the disclosed methods and treatment regimens can be used to treat LSIL. In other aspects, the disclosed methods and treatment regimens can be used to treat CIN. In yet other embodiments, the disclosed methods and treatment regimens can be used to treat ICC. Additionally, the disclosed methods and treatment regimens can be used to inhibit the progression of HPV related dysplasia. In some aspects, for example, the disclosed methods and treatment regimens can be used to inhibit the progression of HSIL. In some aspects, the disclosed methods and treatment regimens can be used to inhibit the progression of ASCUS. In other aspects, the disclosed methods and treatment regimens can be used to inhibit the progression of LSIL. In other aspects, the disclosed methods and treatment regimens can be used to inhibit the progression of CIN. In yet other embodiments, the disclosed methods and treatment regimens can be used to inhibit the progression of ICC.

In one embodiment, there is provided a method of treating a patient having an HPV related dysplasia of the cervix comprising administering intra-vaginally to said patient a therapeutically effective dose of a pharmaceutical composition according to the first aspect. In an alternative embodiment, there is provided a method of treating a patient having an HPV related dysplasia of the cervix comprising administering orally to said patient a therapeutically effective dose of a pharmaceutical composition according to the first aspect.

In one embodiment, the pharmaceutical composition reduces the severity of the HPV related dysplasia. In one embodiment, the severity of the HPV is reduced from CIN3 to CIN2, from CIN3 to CIN1, from CIN3 to HPV negative, from CIN2 to CIN1, from CIN2 to HPV negative, or from CIN1 to HPV negative.

In one embodiment, the patient or subject has a cervical cytology of high grade squamous intraepithelial lesion (HSIL), atypical squamous cells of undetermined significance (ASCUS), or low grade squamous intraepithelial lesion (LSIL).

In one embodiment, the pharmaceutical composition reduces the cervical cytology from HSIL to a normal cytology, from HSIL to ACSUS, from HSIL to LSIL, from ACSUS to a normal cytology, or from LSIL to a normal cytology.

In some embodiments, the composition induces apoptosis of HPV infected cells.

### Process of Manufacture

In a second aspect, there is provided a process to manufacture a self-emulsifying pharmaceutical composition of the first aspect, the process comprising the step of incorporating (e.g. by mixing) at least one active pharmaceutical ingredient (lopinavir), an unsaturated free fatty acid and at least two emulsifiers together to provide the self-emulsifying pharmaceutical composition, wherein the at least two emulsifiers comprise a first emulsifier having a HLB value greater than about 14 and a second emulsifier having a HLB value less than about 6; and wherein the total emulsifier content is less than 30% by weight of the total composition.

In one embodiment, the process comprises a first step comprising incorporating the at least one active pharmaceutical ingredient (lopinavir) into the unsaturated free fatty acid to provide a mixture, followed by a second step comprising incorporating the at least two emulsifiers into the mixture from the first step to provide the self-emulsifying pharmaceutical composition. Therefore, in an embodiment, the process comprises the steps of:
a. incorporating (e.g. by mixing) at least one active pharmaceutical ingredient (lopinavir) in an unsaturated free fatty acid;
b. incorporating (e.g. by mixing) at least two emulsifiers to the mixture from step a) to provide a self-emulsifying composition;
   wherein the at least two emulsifiers comprise a first emulsifier having a HLB value greater than about 14 and a second emulsifier having a HLB value less than about 6; and wherein the total emulsifier content is less than 30% by weight of the total composition.

In an embodiment, the process comprises the steps of:
a. incorporating (e.g. by mixing) lopinavir and a second active pharmaceutical ingredient (such as ritonavir) in an unsaturated free fatty acid;
b. incorporating (e.g. by mixing) at least two emulsifiers to the mixture from step a) to provide a self-emulsifying composition;
   wherein the at least two emulsifiers comprise a first emulsifier having a HLB value greater than about 14 and a second emulsifier having a HLB value less than about 6; and wherein the total emulsifier content is less than 30% by weight of the total composition.

In an embodiment, the process comprises the steps of:
a. incorporating (e.g. by mixing) lopinavir and a second active pharmaceutical ingredient (such as ritonavir) in an unsaturated free fatty acid;
b. incorporating (e.g. by mixing) three emulsifiers to the mixture from step a) to provide a self-emulsifying composition;
   wherein the three emulsifiers comprise a first emulsifier having a HLB value greater than about 14, a second emulsifier having a HLB value less than about 6 and a third emulsifier having a HLB value in the range of about 8 to about 15; and wherein the total emulsifier content is less than 30% by weight of the total composition.

In one embodiment, the first step and/or second step comprises stirring. In one embodiment, the stirring is performed at a rate of at least 10 rpm, such as at least 30 rpm, at least 50 rpm, at least 100 rpm, at least 150 rpm, at least 200 rpm, at least 250 rpm, at least 300 rpm, at least 400 rpm, at least 500 rpm, or such as about 600 rpm. In one embodiment, the stirring in the first step results in at least one active pharmaceutical ingredient dissolving into the unsaturated free fatty acid. In one embodiment, the stirring is performed for 5 minutes after incorporating at least one active pharmaceutical ingredient with the unsaturated free fatty acid together. This advantageously aids with the initial de-clumping of the components of the pharmaceutical composition during this part of the process. In one embodiment, the stirring is repeated every 30 minutes.

In one embodiment, the process is performed at room temperature. In one embodiment, the process is performed at room temperature, and the wt/wt ratio of second emulsifier to first emulsifier present in the composition is between about 1:10 and about 10:1, such as between about 1:5 and about 5:1, between about 1:3 and about 3:1, between about 1:1 and about 5:1, between about 1:1 and about 3:1, between about 1:1 and about 2:1, between about 1:2 and about 2:1, between about 1:1.5 and about 2:1, between about 1:1.3 and about 1:1.1 (such as about 1:1.2), between about 1.1:1 and 1.6:1, between about 1.1:1 and 1.2:1, or between about 1.4:1 and 1.6:1 (such as about 1.5:1). Advantageously, the process is particularly well suited to active pharmaceutical ingredients (such as lopinavir) which are prone to degradation, and wherein the rate and/or extent of degradation is increased when exposed to temperatures above room temperature, for example >30° C., >40° C., >50° C., and >60° C.

In an embodiment, the process comprises the steps of:
a. incorporating (e.g. by mixing) lopinavir and a second active pharmaceutical ingredient (such as ritonavir) in an unsaturated free fatty acid;
b. incorporating (e.g. by mixing) at least two emulsifiers to the mixture from step a) to provide a self-emulsifying composition;
   wherein the at least two emulsifiers comprise a first emulsifier having a HLB value greater than about 14 and a second emulsifier having a HLB value less than about 6; the total emulsifier content is less than 30% by weight of the total composition; and steps a and b are carried out at less than 40° C. (such as less than 30° C.).

In an embodiment, the process comprises the steps of:
a. incorporating (e.g. by mixing) lopinavir and a second active pharmaceutical ingredient (such as ritonavir) in an unsaturated free fatty acid;
b. incorporating (e.g. by mixing) three emulsifiers to the mixture from step a) to provide a self-emulsifying composition;
   wherein the three emulsifiers comprise a first emulsifier having a HLB value greater than about 14, a second emulsifier having a HLB value less than about 6 and a third emulsifier having a HLB value in the range of about 8 to about 15; the total emulsifier content is less than 30% by weight of the total composition; and steps a and b are carried out at less than 40° C. (such as less than 30° C.).

In one embodiment, the process comprises incorporating the at least two emulsifiers, the at least one active pharmaceutical ingredient (lopinavir and ritonavir), and the unsaturated free fatty acid by low shear mixing. In another embodiment, the process comprises incorporating the at least two emulsifiers, the at least one active pharmaceutical ingredient (lopinavir and ritonavir), and the unsaturated free fatty acid by high pressure homogenising.

In one embodiment, the process according to the second aspect is performed under an inert atmosphere. In one embodiment, the inert atmosphere is provided by a vacuum. In a further embodiment, the vacuum is about −0.5 bar. Performing the process under an inert atmosphere has the advantage that any components of the pharmaceutical composition, such as the at least one active pharmaceutical ingredient (lopinavir and/or ritonavir), that are prone to degradation (such as oxidative degradation), are protected from said degradation during the manufacturing process.

In one embodiment, the process according to the second aspect uses an unsaturated free fatty acid which has an assay of at least 95% by weight, such as at least 98% by weight, such as at least 99% by weight, or such as at least 99.5% by weight. Advantageously, using an unsaturated free fatty acid of a high assay level ensures that the pharmaceutical composition is controlled in respect to, for example, the identity, amount and purity of the free fatty acid within the composition.

In an embodiment, the process according to the second aspect further comprises a step of filtering the composition obtained from step b. The filtration may be carried out by any suitable conventional filtration means. In a preferred embodiment, the filtration is carried out by passing the composition through a GAF bag filter.

In an embodiment, the process according to the second aspect further comprises a step of filling the compositions into capsules.

In an embodiment, there is provided a self-emulsifying pharmaceutical composition obtained or obtainable by a process as described herein.

The following numbered statements 1-50 are not claims, but instead define various aspects and embodiments of the invention:
1. A self-emulsifying pharmaceutical composition comprising:
   a. an unsaturated free fatty acid;
   b. at least two emulsifiers; and
   c. at least one active pharmaceutical ingredient (lopinavir);
   wherein the at least two emulsifiers comprise at least a first emulsifier which has a HLB value greater than about 14 and at least a second emulsifier which has a HLB value less than about 6; and wherein the total emulsifier content is less than 30% by weight of the total composition.
2. The pharmaceutical composition according to statement 1, wherein the unsaturated free fatty acid is oleic acid.
3. The pharmaceutical composition according to statement 1 or 2, wherein the at least two emulsifiers is two emulsifiers.
4. The pharmaceutical composition according to any preceding statement, wherein the first emulsifier has a HLB value greater than 14 and the second emulsifier has a HLB value less than 6.
5. The pharmaceutical composition according to any preceding statement, wherein the first emulsifier has a HLB value greater than about 15, such as greater than about 16, greater than about 17, or greater than about 18.
6. The pharmaceutical composition according to any preceding statement, wherein the first emulsifier is a solid at room temperature.
7. The pharmaceutical composition according to any preceding statement, wherein the first emulsifier is a polyol ester, such as a polyol stearate (for example PEG100 stearate).
8. The pharmaceutical composition according statements 1-5, wherein the first emulsifier is a liquid at room temperature.
9. The pharmaceutical composition according to statement 8, wherein the first emulsifier is a polyethoxylated sorbitan ester, such as polysorbate 20, polysorbate 60 or polysorbate 80.
10. The pharmaceutical composition according to any preceding statement, wherein the second emulsifier has a HLB value less than about 5.5, such as less than about 5, less than about 4.5, or less than about 4.
11. The pharmaceutical composition according to any preceding statement, wherein the second emulsifier is a semi-solid at room temperature.
12. The pharmaceutical composition according to any preceding statement, wherein the second emulsifier is a monoglyceride, such as glycerol monooleate.
13. The pharmaceutical composition according to any preceding statement, wherein the wt/wt ratio of second emulsifier to first emulsifier present in the composition is between about 1:10 and about 10:1, such as between about 1:5 and about 5:1, between about 1:3 and about 3:1, between about 1:1 and about 5:1, between about 1:1 and about 3:1, between about 1:1 and about 2:1, between about 1:2 and about 2:1, between about 1:1.5 and about 2:1, between about 1:1.3 and about 1:1.1 (such as about 1:1.2), or between about 1.4:1 and 1.6:1 (such as about 1.5:1).
14. The pharmaceutical composition according to any preceding statement, wherein the at least two emulsifiers is three emulsifiers.
15. The pharmaceutical composition according to statement 14, wherein the first emulsifier has a HLB value greater than 14, the second emulsifier has a HLB value less than 6, the third emulsifier has a HLB value in the range 8 to 15; and the total emulsifier content is less than 30% by weight of the total composition.
16. The pharmaceutical composition according to statement 15, wherein the third emulsifier has a HLB value in the range 10 to 15, such as 11 to 15, 12 to 15, 13 to 15, 11 to 14 or 12 to 14.
17. The pharmaceutical composition according to statements 15 or 16, wherein the third emulsifier is a polyoxyl castor oil derivative (such as PEG 35 castor oil).
18. The pharmaceutical composition according to any preceding statement, wherein the total emulsifier content is less than 25% by weight, less than 20% by weight, less than 15% by weight, 2% to 20% by weight, 2.5% to 15% by weight, 5% to 15% by weight, 8% to 12%, 10% to 20%, 12% to 20%, 10% to 18%, 12% to 18%, 12% to 16%, 13% to 20%, 14% to 20%, 15% to 20%, 13% to 25%, 14% to 25%, 15% to 25%, 20% to 25%, 20% to 26%, 20% to 27%, 20% to 28% or 20% to 29% by weight of the total composition.
19. The pharmaceutical composition according to any preceding statement, wherein the unsaturated free fatty acid is present in the pharmaceutical composition at a level of at least 25% by weight of the total pharmaceutical composition, such as at least 35% by weight, at least 45% by weight, about 50 to about 85% by weight, about 50 to about 75% by weight, about 50 to about 70% by weight, about 60 to about 80% by weight, about 65 to about 80% by weight, about 65 to about 75% by weight, or about 68 to about 72% by weight of the total composition.
20. The pharmaceutical composition according to any preceding statement, wherein the first emulsifier is present in the pharmaceutical composition at about 1% to about 20% by weight of the total pharmaceutical composition, such as about 1% to about 10% by weight, about 10% to about 20% by weight, about 10% to about 15% by weight, about 1% to about 5% by weight, about 3% to about 7% by weight, about 3% to about 6% by weight, about 3% to about 5% by weight (such as about 4% by weight), about 4% to about 5% by weight (such as about 4.3% by weight), or about 5% to about 6% by weight (such as about 5.5% by weight) of the total composition.
21. The pharmaceutical composition according to any preceding statement, wherein the second emulsifier is present in the pharmaceutical composition at about 1% to about 20% by weight of the total pharmaceutical composition, such as about 1% to about 10% by weight, about 2% to about 8% by weight, about 4% to about 7% by weight, about 5% to about 7% by weight (such as about 6% by weight), about 4% to about 6% by weight (such as about 5% by weight),or about 4% to about 5% by weight (such as about 4.5% by weight) of the total composition.
22. The pharmaceutical composition according to any one of statements 14-21, wherein the third emulsifier is present in the pharmaceutical composition at about 1% to about 10% by weight of the total pharmaceutical composition, such as about 2% to about 8% by weight, about 3% to about 7% by weight, about 4% to about 6% by weight, or about 5% by weight of the total composition.
23. The pharmaceutical composition according to any preceding statement, wherein the at least one active pharmaceutical ingredient (lopinavir) is present in a dissolved state.
24. The pharmaceutical composition according to any preceding statement, wherein the at least one active pharmaceutical ingredient (lopinavir) is stable within the pharmaceutical composition.
25. The pharmaceutical composition according to any preceding statement, wherein the at least one active pharmaceutical ingredient has a solubility of at least 1% w/v in the unsaturated free fatty acid.
26. The pharmaceutical composition according to any preceding statement, wherein the at least one active pharmaceutical ingredient has an aqueous solubility of less than 0.1 mg/ml, such as less than 0.01 mg/ml.
27. The pharmaceutical composition according to any preceding statement, wherein the at least one active pharmaceutical ingredient has a log P value greater than 4, such as greater than 5.
28. The pharmaceutical composition according to any preceding statement, wherein the at least one active pharmaceutical ingredient is a HIV protease enzyme inhibitor.
29. The pharmaceutical composition according to statement 28, wherein the HIV protease enzyme inhibitor is selected from lopinavir and ritonavir.
30. The pharmaceutical composition according to any preceding statement, wherein the at least one active pharmaceutical ingredient is two active pharmaceutical ingredients.
31. The pharmaceutical composition according to any preceding statement, wherein the pharmaceutical composition comprises lopinavir and ritonavir.
32. The pharmaceutical composition according to statement 31, wherein the molar ratio of lopinavir to ritonavir present in the composition is between about 1:10 and about 18:1, such as between about 1:10 and about 15:1, such as between about 1:5 and about 15:1, such as between about 1:1 and about 15:1, such as between about 2:1 and about 15:1, such as between about 4:1 and about 15:1, such as between about 8:1 and about 14:1, such as between about 9:1 and about 14:1, such as between about 10:1 and about 14:1, such as between 10.5:1 and about 18:1, such as between 10.5:1 and 18:1, such as between about 10.5:1 and about 14:1, such as between about 11:1 to about 13:1, such as between about 11.5 and about 17:1, such as between about 11.5:1 and about 16.0:1, such as between about 11.5:1 and about 15:1, such as about 14.5:1, such as 14.5:1, such as about 14:1, such as 14:1, such as about 13.8:1, such as 13.8:1, such as about 13.75:1, such as 13.75:1, such as about 13.5:1, such as 13.5:1, such as about 13:1, such as 13:1, such as about 12.5:1, such as 12.5:1, such as about 12:1, such as 12:1, such as about 11.75:1, such as 11.75:1, such as about 9:1, such as 9:1, such as about 5:1, such as 5:1, such as about 4.6:1, or such as 4.6:1.
33. The pharmaceutical composition according to statement 31, wherein the wt/wt ratio of lopinavir to ritonavir present in the composition is between about 1:10 and about 18:1, such as between about 1:10 and about 15:1, such as between about 1:5 and about 15:1, such as between about 1:1 and about 15:1, such as between about 2:1 and about 15:1, such as between about 4:1 and about 15:1, such as between about 8:1 and about 14:1, such as between about 9:1 and about 14:1, such as between about 10:1 and about 14:1, such as between 10.5:1 and about 18:1, such as between 10.5:1 and 18:1, such as between about 10.5:1 and about 14:1, such as between about 11:1 to about 13:1, such as between about 11.5 and about 17:1, such as between about 11.5:1 and about 16.0:1, such as between about 11.5:1 and about 15:1, such as about 14.5:1, such as 14.5:1, such as about 14:1, such as 14:1, such as about 13.8:1, such as 13.8:1, such as about 13.75:1, such as 13.75:1, such as about 13.5:1, such as 13.5:1, such as about 13:1, such as 13:1, such as about 12.5:1, such as 12.5:1, such as about 12:1, such as 12:1, such as about 11.75:1, such as 11.75:1, such as about 11.5:1, such as 11.5:1, such as about 11.25:1, such as 11.25:1, or such as about 11:1, such as 11:1.
34. The pharmaceutical composition according to any preceding statement, further comprising an antioxidant, such as butylated hydroxytoluene.
35. A self-emulsifying pharmaceutical composition consisting of:
   a. an unsaturated free fatty acid;
   b. a first emulsifier having a HLB value greater than about 14;
   c. a second emulsifier having a HLB value less than about 6;
   d. an antioxidant; and
   e. at least one active pharmaceutical ingredient (lopinavir);
   wherein the total emulsifier content is less than 30% by weight of the total composition.
36. A self-emulsifying pharmaceutical composition consisting of:
   a. an unsaturated free fatty acid;
   b. a first emulsifier having a HLB value greater than about 14;
   c. a second emulsifier having a HLB value less than about 6;
   d. a third emulsifier;
   e. an antioxidant;
   f. lopinavir; and
   g. ritonavir;
   wherein the total emulsifier content is less than 30% by weight of the total composition.
37. The pharmaceutical composition according to any preceding statement wherein the pharmaceutical composition is encapsulated within a capsule.
38. The pharmaceutical composition according to any preceding statement having a dissolution profile, when the composition is filled in hard gel capsules with sinkers and measured in 0.7% w/v SLS media at about 37° C. using USP apparatus II at 25 rpm for 0-60 minutes and 200 rpm for 60-75 minutes, which comprises:
   greater than 20% API dissolution after 10 minutes, such as greater than 40% API dissolution after 10 minutes.
39. The pharmaceutical composition according to any preceding statement having a dissolution profile, when the composition is filled in hard gel capsules with sinkers and measured in 0.7% w/v SLS media at about 37° C. using USP apparatus II at 25 rpm for 0-60 minutes and 200 rpm for 60-75 minutes, which comprises:
   greater than 60% API dissolution after 45 minutes, such as greater than 70% API dissolution after 45 minutes.
40. The pharmaceutical composition according to any preceding statement having a dissolution profile, when the composition is filled in hard gel capsules with sinkers and measured in 0.7% w/v SLS media at about 37° C. using USP apparatus II at 25 rpm for 0-60 minutes and 200 rpm for 60-75 minutes, which comprises:
   greater than 90% API dissolution after 75 minutes, such as greater than 95% API dissolution after 75 minutes.
41. The pharmaceutical composition according to any preceding statement for use as a medicament.
42. A method of treating and/or inhibiting the development or progression of cancers and benign proliferative disorders in a subject in need of such treatment or inhibition comprising administering a therapeutically effective amount of a pharmaceutical composition according to any of statements 1-40 to said subject.
43. The method according to statement 42, wherein the cancer or disorder is caused or induced by a human papilloma virus (HPV).
44. A method of treating a patient having an HPV related dysplasia of the cervix comprising administering intra-vaginally or orally to said patient a therapeutically effective dose of a pharmaceutical composition according to any one of statements 1-40.
45. The method of statement 44, wherein the pharmaceutical composition reduces the severity of the HPV related dysplasia.
46. The method of statement 45, wherein the severity of the HPV is reduced from CIN3 to CIN2, from CIN3 to CIN1, from CIN3 to HPV negative, from CIN2 to CIN1, from CIN2 to HPV negative, or from CIN1 to HPV negative.
47. The method of any one of statements 43-46, wherein the composition induces apoptosis of HPV infected cells.
48. The method of any one of statements 43-47, wherein the patient has a cervical cytology of high grade squamous intraepithelial lesion (HSIL), atypical squamous cells of undetermined significance (ASCUS), or low grade squamous intraepithelial lesion (LSIL).
49. The method of statement 48, wherein the composition reduces the cervical cytology from HSIL to a normal cytology, from HSIL to ACSUS, from HSIL to LSIL, from ACSUS to a normal cytology, or from LSIL to a normal cytology.
50. A process to manufacture a self-emulsifying pharmaceutical composition of any one of statements 1 to 40, the process comprising the steps of:
   a. incorporating (e.g. by mixing) at least one active pharmaceutical ingredient (lopinavir) in an unsaturated free fatty acid;
   b. incorporating (e.g. by mixing) at least two emulsifiers to the mixture from step a) to provide a self-emulsifying composition;
wherein the at least two emulsifiers comprise a first emulsifier having a HLB value greater than about 14 and a second emulsifier having a HLB value less than about 6; and wherein the total emulsifier content is less than 30% by weight of the total composition.

### EXAMPLES

Particular embodiments of the invention are further described hereinafter, with reference to the accompanying drawings, in which:

FIG. 1 shows the lopinavir dissolution profiles of Formulations 3a, 3b and 3c in SLS media (25 rpm).

FIG. 2 shows the lopinavir dissolution profile of Formulation 3a compared with the lopinavir dissolution profiles of Comparative Formulation 3f and Comparative Formulation 3h in SLS media (25 rpm).

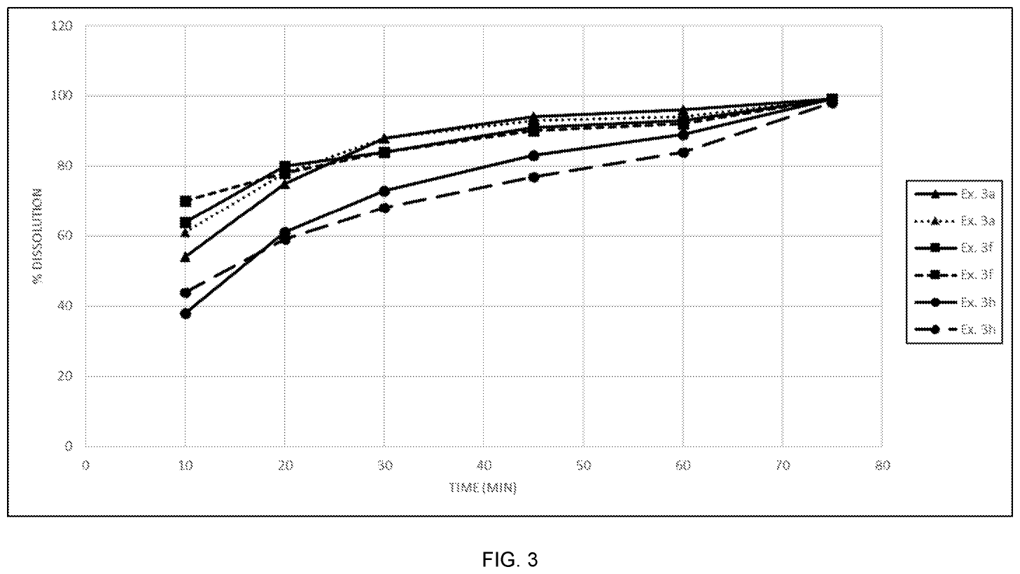

FIG. 3 shows the ritonavir dissolution profile of Formulation 3a compared with the ritonavir dissolution profiles of Comparative Formulation 3f and Comparative Formulation 3h in SLS media (25 rpm).

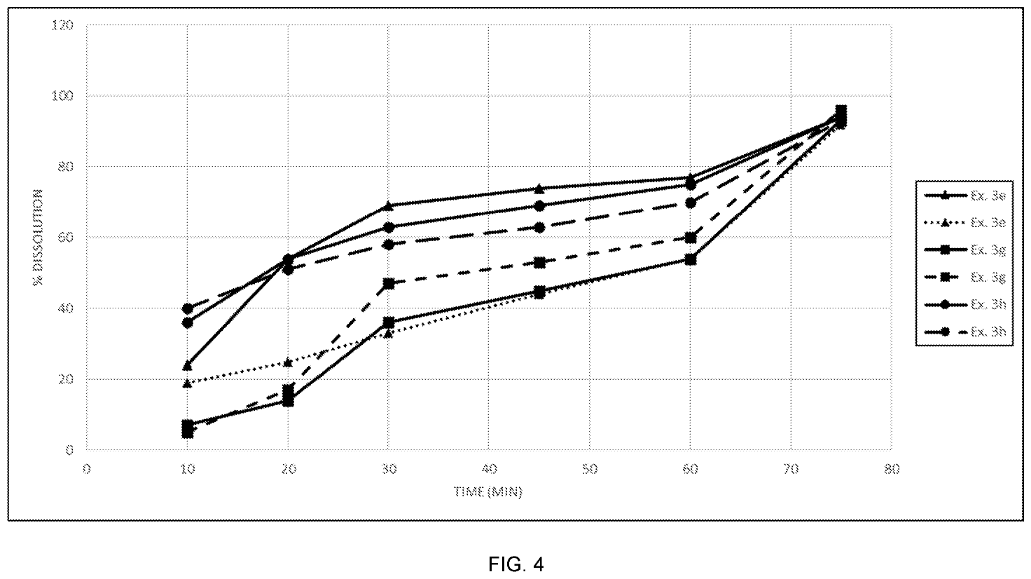

FIG. 4 shows the lopinavir dissolution profiles of Comparative Formulations 3e, 3 g and 3h in SLS media (25 rpm).

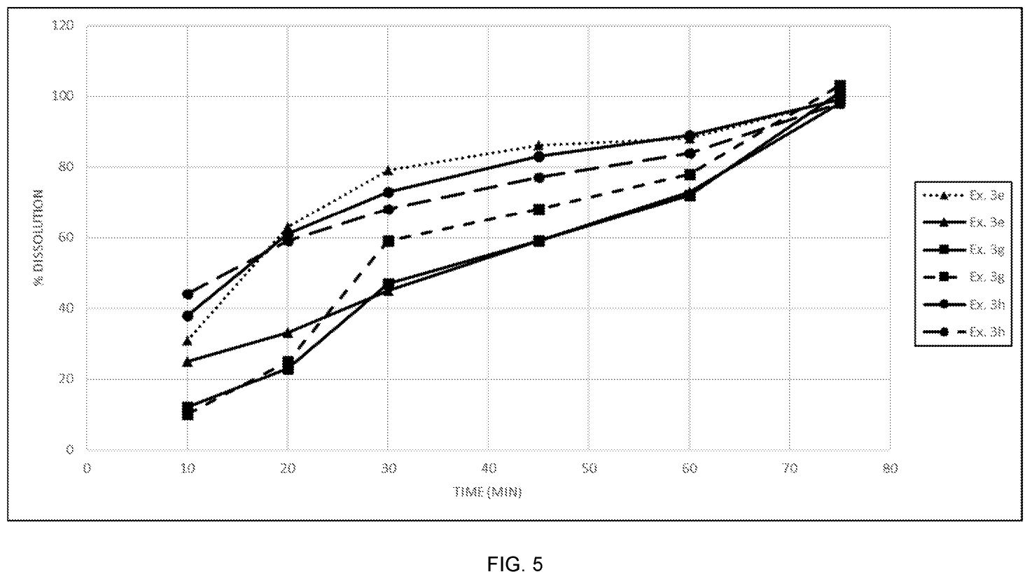

FIG. 5 shows the ritonavir dissolution profiles of Comparative Formulations 3e, 3 g and 3h in SLS media (25 rpm).

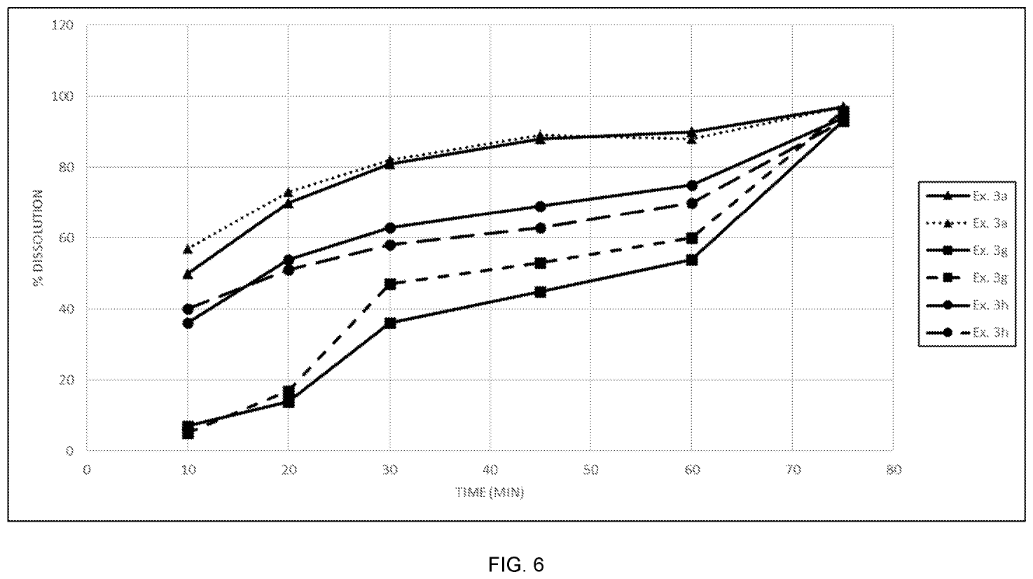

FIG. 6 shows the lopinavir dissolution profile of Formulation 3a compared with the lopinavir dissolution profiles of Comparative Formulation 3 g and Comparative Formulation 3h in SLS media (25 rpm).

FIG. 7 shows the lopinavir dissolution profile of Formulation 3a compared with the dissolution profiles of Comparative Formulation 1 (Example 4) and Comparative Formulation 2 (Example 5) in SLS media (25 rpm).

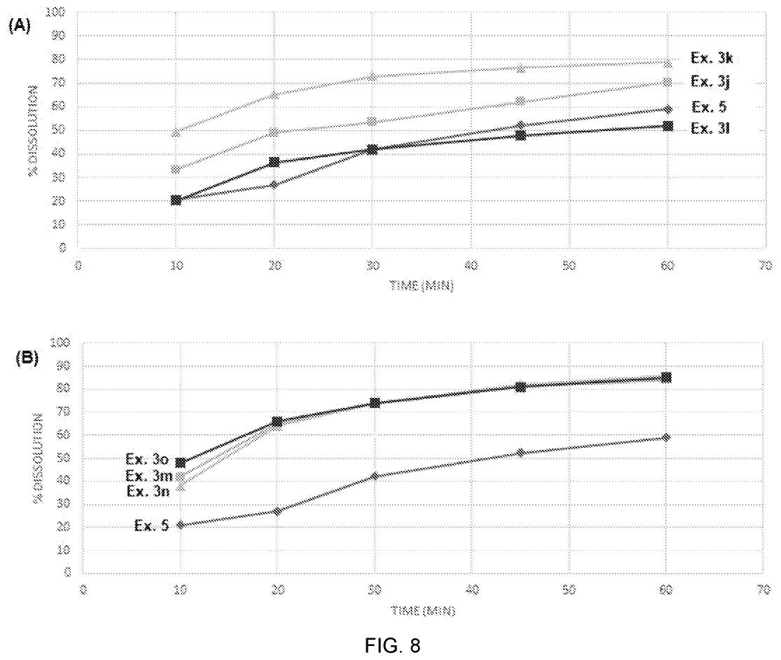

FIG. 8 shows the lopinavir dissolution profiles in SLS media (50 rpm) of (A) Formulation 3j compared with the dissolution profiles of Comparative Formulations 3k, 3l and Comparative Formulation 2 (Example 5); and (B) Formulations 3m, 3n and 3o compared with the dissolution profile of Comparative Formulation 2 (Example 5).

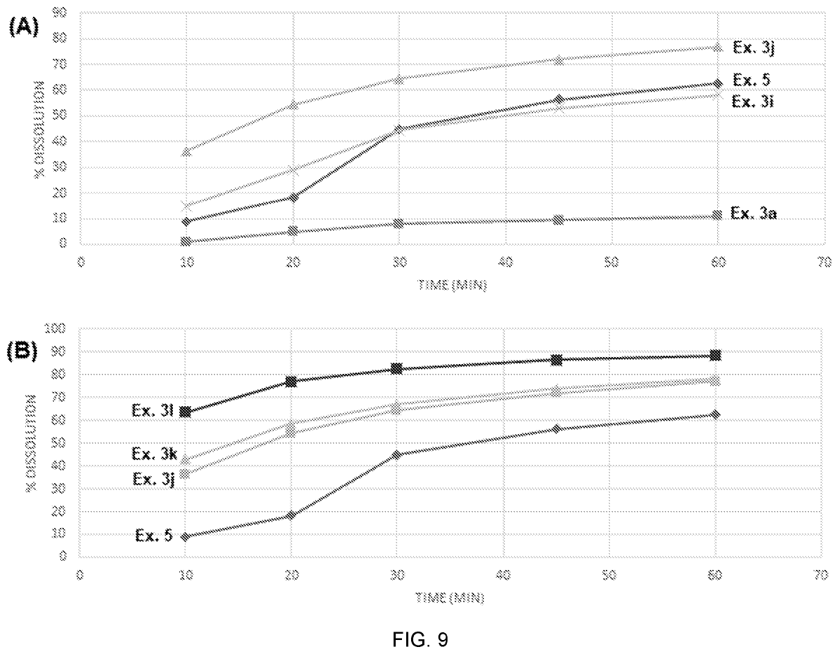

FIG. 9 shows the lopinavir dissolution profiles in CTAB media (50 rpm) of (A) Formulations 3a, 3i and 3j compared with the dissolution profile of Comparative Formulation 2 (Example 5); and (B) Formulation 3j compared with the dissolution profiles of Comparative Formulations 3k, 31 and Comparative Formulation 2 (Example 5).

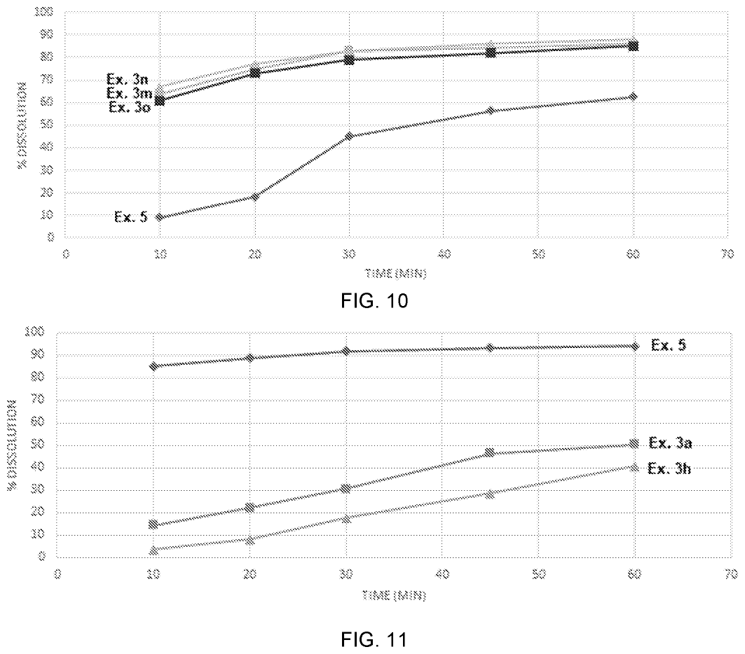

FIG. 10 shows the lopinavir dissolution profiles in CTAB media (50 rpm) of Formulations 3m, 3n and 3o compared with the dissolution profile of Comparative Formulation 2 (Example 5).

FIG. 11 shows the lopinavir dissolution profiles in Brij®10 media (50 rpm) of Formulation 3a compared with the dissolution profile of Comparative Formulation 3h and Comparative Formulation 2 (Example 5).

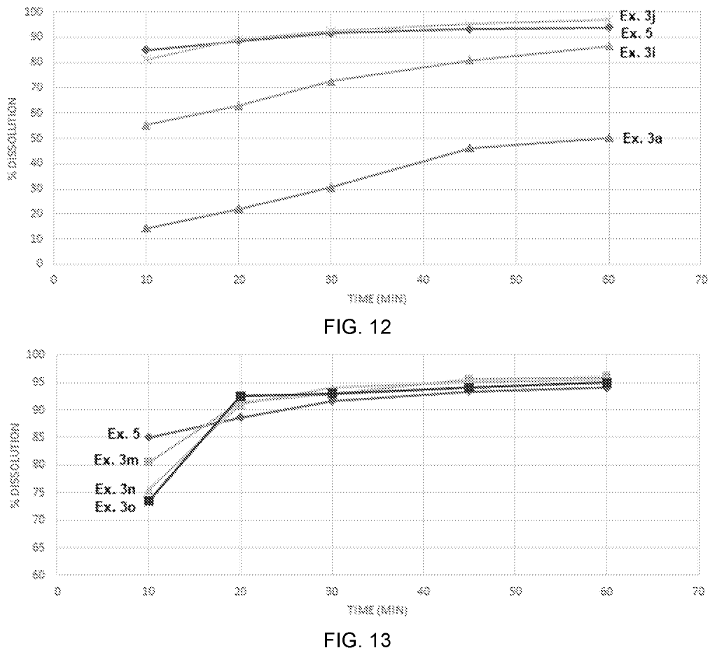

FIG. 12 shows the lopinavir dissolution profiles in Brij®10 media (50 rpm) of Formulations 3a, 3i and 3j compared with the dissolution profile of Comparative Formulation 2 (Example 5).

FIG. 13 shows the lopinavir dissolution profiles in Brij®10 media (50 rpm) of Formulations 3m, 3n and 3o compared with the dissolution profile of Comparative Formulation 2 (Example 5).

The following abbreviations have been used in the Examples:
API—active pharmaceutical ingredient
HPMC—hydroxypropylmethylcellulose
PEG—polyethylene glycol

### Example 1: Solubility of Ritonavir and/or Lopinavir

The solubility of Ritonavir and Lopinavir, and Ritonavir alone in a range of pharmaceutically acceptable solvents is presented below. As can be seen, Ritonavir and Lopinavir, and Ritonavir alone have high solubility in oleic acid. P22,TI

| Solvent/Solvent system | Solubility (% w/v) |
|---|---|
| Oleic acid | ~18^{1, 2} |
| Ethyl linoleate³ | 0.55 |
| Ethanol³ | >15 |
| PEG-35 castor oil³ | 9.73 |
| Oleic acid³ | >15 |
| Ethyl linoleate:oleic acid (3:1)³ | 14.2 |
| Ethyl linoleate:PEG-35 castor oil (1:1)³ | 7.93 |

¹Empirically determined, by mass weighing, at ambient temperature, using Lopinavir/Ritonavir API blend molar ratio of 9:1
²Based on oleic acid specific gravity of 0.9
³Solubility for Ritonavir only, measured at 37° C., Lei et al. “Development of a novel self-micro-emulsifying drug delivery system (SMEDDS) for reducing HIV protease inhibitor-induced intestinal epithelial barrier dysfunction”, Mol. Pharm. 2010 June 7:7(3) 844-853

### Example 2: Ritonavir and Lopinavir Chemical and Physical Stability

It is known from the literature that potency loss for Lopinavir and Ritonavir is thermolytically driven and Ritonavir is particularly prone to hydrolysis due to its carbamate functional group (Donato et al, “LC Method for Studies on the Stability of Lopinavir and Ritonavir in Soft Gelatin Capsules”, Chromatographia, April 2006, 63, 437-443). Ritonavir is also known to be oxygen labile. A preferable manufacturing route for compositions comprising Lopinavir/Ritonavir is thus one that excludes heat, water, and/or oxygen.

### Example 3: Preparation of Formulations

For all formulations present below, all materials used are pharmaceutical grade (either US Pharmacopeia or European Pharmacopeia) except for white ceresin wax, which is Japanese Pharmaceutical Excipient grade. The oleic acid used was super refined grade (Supplier: Croda).

### General Procedure

The manufacture of the formulations 3a-3h was carried according to the following general procedure, with the component amounts specified in Tables 1 to 8 below.

The oleic acid and lopinavir bulk supplies were purged with nitrogen after each use. Ritonavir was protected from UV light.
i. Into a main vessel was added butylated hydroxytoluene, lopinavir and ritonavir into oleic acid. The mixture was stirred to dissolve the APIs at 600 rpm.
ii. To the main vessel was added polyoxyl 100 stearate and glycerol monooleate. The mixture was further stirred to dissolve the emulsifiers at 600 rpm.
iii. The product was discharge from the vessel and passed through a GAF bag filter.
iv. The filtered product was packed into a glass jar, which was then stored in the dark at 0-5° C.
v. As required, the formulation (800 mg) was then filled into hard gelatin capsules and sealed with ethanol.

TABLE 1
Formulation 3a
Batch number: R131/F099
Batch size: 200 g
Active supplier: Cipla

| | | ID | MP | | | |
|---|---|---|---|---|---|---|
| # | Ingredient | number | ° C. | Function | % w/w | g/batch |
| 1 | Oleic Acid | 101977 | 13 | Solvent | 69.487 | 138.97 |
| 2 | Polyoxyl 100 | 102002 | 54-59 | Emulsifier | 4.00 | 8.00 |
| | stearate | | | | | |
| 3 | Glycerol | 101976 | 36-40 | Emulsifier | 6.00 | 12.00 |
| | monooleate | | | | | |
| 4 | Butylated | 100826 | 70 | Antioxidant | 0.20 | 0.40 |
| | hydroxytoluene | | | | | |
| 5 | Lopinavir | 102043 | 124 | API | 18.750 | 37.50 |
| 6 | Ritonavir | 102044 | 120 | API | 1.5625 | 3.13 |
| | | | | synergist | | |
| | | | | TOTAL | 100.00 | 200 |

The viscosity of the liquid formulation 3a was measured on a Brookfield DV-II+ Pro Viscometer, using a 10 g sample, SC4-34 spindle, 60 rpm at 25° C. The sample was found to have a dynamic viscosity of 130 cP.s and 13.0% torque.

TABLE 2
Formulation 3b
Batch number: R131/F097
Batch size: 200 g
Active supplier: Cipla

| | | ID | MP | | | |
|---|---|---|---|---|---|---|
| # | Ingredient | number | ° C. | Function | % w/w | g/batch |
| 1 | Oleic Acid | 101977 | 13 | Solvent | 74.487 | 148.97 |
| 2 | Polyoxyl 100 | 102002 | 54-59 | Emulsifier | 2.00 | 4.00 |
| | stearate | | | | | |
| 3 | Glycerol | 101976 | 36-40 | Emulsifier | 3.00 | 6.00 |
| | monooleate | | | | | |
| 4 | Butylated | 100826 | 70 | Antioxidant | 0.20 | 0.40 |
| | hydroxytoluene | | | | | |
| 5 | Lopinavir | 102043 | 124 | API | 18.750 | 37.50 |
| 6 | Ritonavir | 102044 | 120 | API | 1.5625 | 3.13 |
| | | | | synergist | | |
| | | | | TOTAL | 100.00 | 200 |

The viscosity of the liquid formulation 3b was measured on a Brookfield DV-II+ Pro Viscometer, using a 10 g sample, SC4-34 spindle, 60 rpm at 25° C. The sample was found to have a dynamic viscosity of 138 cP.s and 13.8% torque.

TABLE 3
Formulation 3c
Batch number: R131/F101
Batch size: 200 g
Active supplier: Cipla

| | | ID | MP | | | |
|---|---|---|---|---|---|---|
| # | Ingredient | number | ° C. | Function | % w/w | g/batch |
| 1 | Oleic Acid | 101977 | 13 | Solvent | 77.000 | 154.00 |
| 2 | Polyoxyl 100 | 102002 | 54-59 | Emulsifier | 1.00 | 2.00 |
| | stearate | | | | | |
| 3 | Glycerol | 101976 | 36-40 | Emulsifier | 1.50 | 3.00 |
| | monooleate | | | | | |
| 4 | Butylated | 100826 | 70 | Antioxidant | 0.20 | 0.40 |
| | hydroxytoluene | | | | | |
| 5 | Lopinavir | 102043 | 124 | API | 18.750 | 37.50 |
| 6 | Ritonavir | 102044 | 120 | API | 1.5625 | 3.13 |
| | | | | synergist | | |
| | | | | TOTAL | 100.01 | 200 |

The viscosity of the liquid formulation 3c was measured on a Brookfield DV-II+ Pro Viscometer, using a 10 g sample, SC4-34 spindle, 60 rpm at 25° C. The sample was found to have a dynamic viscosity of 125 cP.s and 12.5% torque.

TABLE 4
Formulation 3d
Batch number: R131/F102
Batch size: 40 kg
Active supplier: Cipla

| | | ID | MP | | | |
|---|---|---|---|---|---|---|
| # | Ingredient | number | ° C. | Function | % w/w | kg/batch |
| 1 | Oleic Acid | 101977 | 13 | Solvent | 71.74 | 28.696 |
| 2 | Polyoxyl 100 | 102002 | 54-59 | Emulsifier | 4.00 | 1.60 |
| | stearate | | | | | |
| 3 | Glycerol | 101976 | 36-40 | Emulsifier | 6.00 | 2.40 |
| | monooleate | | | | | |
| 4 | Butylated | 100826 | 70 | Antioxidant | 0.20 | 0.08 |
| | hydroxytoluene | | | | | |
| 5 | Lopinavir | 102043 | 124 | API | 16.67 | 6.668 |
| 6 | Ritonavir | 102044 | 120 | API | 1.39 | 0.556 |
| | | | | synergist | | |
| | | | | TOTAL | 100.00 | 40 |

TABLE 5
Comparative Formulation 3e
Batch number: R131/F107
Batch size: 200 g
Active supplier: Cipla

| | | ID | MP | | | |
|---|---|---|---|---|---|---|
| # | Ingredient | number | ° C. | Function | % w/w | g/batch |
| 1 | Oleic Acid | 101977 | 13 | Solvent | 77.49 | 154.97 |
| 2 | Polyoxyl 100 | 102002 | 54-59 | Emulsifier | 2.00 | 4.00 |
| | stearate | | | | | |
| 4 | Butylated | 100826 | 70 | Antioxidant | 0.20 | 0.40 |
| | hydroxytoluene | | | | | |
| 5 | Lopinavir | 102043 | 124 | API | 18.75 | 37.50 |
| 6 | Ritonavir | 102044 | 120 | API | 1.56 | 3.13 |
| | | | | synergist | | |
| | | | | TOTAL | 100.00 | 200.00 |

TABLE 6
Comparative Formulation 3f
Batch number: R131/F108
Batch size: 200 g
Active supplier: Cipla

| | | ID | MP | | | |
|---|---|---|---|---|---|---|
| # | Ingredient | number | ° C. | Function | % w/w | g/batch |
| 1 | Oleic Acid | 101977 | 13 | Solvent | 76.49 | 152.97 |
| 3 | Glycerol | 101976 | 36-40 | Emulsifier | 3.00 | 6.00 |
| | monooleate | | | | | |
| 4 | Butylated | 100826 | 70 | Antioxidant | 0.20 | 0.40 |
| | hydroxytoluene | | | | | |
| 5 | Lopinavir | 102043 | 124 | API | 18.75 | 37.50 |
| 6 | Ritonavir | 102044 | 120 | API | 1.56 | 3.13 |
| | | | | synergist | | |
| | | | | TOTAL | 100.00 | 200.00 |

TABLE 7
Comparative Formulation 3g
Batch number: R131/F109
Batch size: 200 g
Active supplier: Cipla

| | | ID | MP | | | |
|---|---|---|---|---|---|---|
| # | Ingredient | number | ° C. | Function | % w/w | g/batch |
| 1 | Oleic Acid | 101977 | 13 | Solvent | 69.49 | 138.97 |
| 2 | Polyoxyl 100 | 102002 | 54-59 | Emulsifier | 10.00 | 20.00 |
| | stearate | | | | | |
| 4 | Butylated | 100826 | 70 | Antioxidant | 0.20 | 0.40 |
| | hydroxytoluene | | | | | |
| 5 | Lopinavir | 102043 | 124 | API | 18.75 | 37.50 |
| 6 | Ritonavir | 102044 | 120 | API | 1.56 | 3.13 |
| | | | | synergist | | |
| | | | | TOTAL | 100.00 | 200.00 |

TABLE 8
Comparative Formulation 3h
Batch number: R131/F110
Batch size: 200 g
Active supplier: Cipla

| | | ID | MP | | | |
|---|---|---|---|---|---|---|
| # | Ingredient | number | ° C. | Function | % w/w | g/batch |
| 1 | Oleic Acid | 101977 | 13 | Solvent | 69.49 | 138.97 |
| 3 | Glycerol | 101976 | 36-40 | Emulsifier | 10.00 | 20.00 |
| | monooleate | | | | | |
| 4 | Butylated | 100826 | 70 | Antioxidant | 0.20 | 0.40 |
| | hydroxytoluene | | | | | |
| 5 | Lopinavir | 102043 | 124 | API | 18.75 | 37.50 |
| 6 | Ritonavir | 102044 | 120 | API | 1.56 | 3.13 |
| | | | | synergist | | |
| | | | | TOTAL | 100.00 | 200.00 |

The manufacture of the formulations 3i-3t was carried according to the following general procedure, with the component amounts specified in Tables 9 to 20 below.

The oleic acid and lopinavir bulk supplies were purged with nitrogen after each use. Ritonavir was protected from UV light.
i. Into a main vessel was added lopinavir and ritonavir to butylated hydroxytoluene dissolved in oleic acid. The mixture was stirred to dissolve the APIs at 600 rpm.
ii. To the main vessel was added PEG 35 castor oil, glycerol monooleate and polyoxyl 100 stearate/polysorbate 20/polysorbate 60/polysorbate 80 (as applicable). The mixture was further stirred to dissolve the emulsifiers at 600 rpm.
iii. The product was discharge from the vessel and passed through a GAF bag filter.
iv. The filtered product was packed into a glass jar, which was then stored in the dark at 0-5° C.
v. As required, the formulation (800 mg) was then filled into hard gelatin capsules and sealed with ethanol.

TABLE 9
Formulation 3i
Batch number: F120
Batch size: 200 g
Active supplier: Cipla

| | | ID | MP | | | |
|---|---|---|---|---|---|---|
| # | Ingredient | number | ° C. | Function | % w/w | g/batch |
| 1 | Oleic Acid | 101977 | 13 | Solvent | 66.987 | 133.97 |
| 2 | Polyoxyl 100 | 102002 | 54-59 | Emulsifier | 4.00 | 8.00 |
| | stearate | | | | | |
| 3 | Glycerol | 101976 | 36-40 | Emulsifier | 6.00 | 12.00 |
| | monooleate | | | | | |
| 4 | PEG-35 | 102491 | −4 | Surfactant | 2.50 | 5.00 |
| | castor oil | | | | | |
| 5 | Butylated | 100826 | 70 | Antioxidant | 0.20 | 0.40 |
| | hydroxytoluene | | | | | |
| 6 | Lopinavir | 102043 | 124 | API | 18.750 | 37.50 |
| 7 | Ritonavir | 102044 | 120 | API | 1.5625 | 3.13 |
| | | | | synergist | | |
| | | | | TOTAL | 100.00 | 200.0 |

TABLE 10
Formulation 3j
Batch number: F135
Batch size: 200 g
Active supplier: Cipla

| | | ID | MP | | | |
|---|---|---|---|---|---|---|
| # | Ingredient | number | ° C. | Function | % w/w | g/batch |
| 1 | Oleic Acid | 101977 | 13 | Solvent | 66.740 | 133.48 |
| 2 | Polyoxyl 100 | 102002 | 54-59 | Emulsifier | 4.00 | 8.00 |
| | stearate | | | | | |
| 3 | Glycerol | 101976 | 36-40 | Emulsifier | 6.00 | 12.00 |
| | monooleate | | | | | |
| 4 | PEG-35 castor oil | 102491 | −4 | Surfactant | 5.00 | 10.00 |
| 5 | Butylated | 100826 | 70 | Antioxidant | 0.20 | 0.40 |
| | hydroxytoluene | | | | | |
| 6 | Lopinavir | 102043 | 124 | API | 16.67 | 33.34 |
| 7 | Ritonavir | 102044 | 120 | API | 1.39 | 2.78 |
| | | | | synergist | | |
| | | | | TOTAL | 100.00 | 200.0 |

TABLE 11
Comparative Formulation 3k
Batch number: F136
Batch size: 200 g
Active supplier: Cipla

| | | ID | | | | |
|---|---|---|---|---|---|---|
| # | Ingredient | number | MP ° C. | Function | % w/w | g/batch |
| 1 | Oleic Acid | 101977 | 13 | Solvent | 70.740 | 141.48 |
| 2 | Glycerol | 101976 | 36-40 | Emulsifier | 6.00 | 12.00 |
| | monooleate | | | | | |
| 3 | PEG-35 | 102491 | −4 | Surfactant | 5.00 | 10.00 |
| | castor oil | | | | | |
| 4 | Butylated | 100826 | 70 | Antioxidant | 0.20 | 0.40 |
| | hydroxytoluene | | | | | |
| 5 | Lopinavir | 102043 | 124 | API | 16.67 | 33.34 |
| 6 | Ritonavir | 102044 | 120 | API | 1.39 | 2.78 |
| | | | | synergist | | |
| | | | | TOTAL | 100.00 | 200.0 |

TABLE 12
Comparative Formulation 3l
Batch number: F137
Batch size: 200 g
Active supplier: Cipla

| | | ID | | | | |
|---|---|---|---|---|---|---|
| # | Ingredient | number | MP ° C. | Function | % w/w | g/batch |
| 1 | Oleic Acid | 101977 | 13 | Solvent | 72.740 | 145.48 |
| 2 | Polyoxyl 100 | 102002 | 54-59 | Emulsifier | 4.00 | 8.00 |
| | stearate | | | | | |
| 3 | PEG-35 | 102491 | −4 | Surfactant | 5.00 | 10.00 |
| | castor oil | | | | | |
| 4 | Butylated | 100826 | 70 | Antioxidant | 0.20 | 0.40 |
| | hydroxytoluene | | | | | |
| 5 | Lopinavir | 102043 | 124 | API | 16.67 | 33.34 |
| 6 | Ritonavir | 102044 | 120 | API | 1.39 | 2.78 |
| | | | | synergist | | |
| | | | | TOTAL | 100.00 | 200.0 |

TABLE 13
Formulation 3m
Batch number: F138
Batch size: 200 g
Active supplier: Cipla

| | | ID | | | | |
|---|---|---|---|---|---|---|
| # | Ingredient | number | MP ° C. | Function | % w/w | g/batch |
| 1 | Oleic Acid | 101977 | 13 | Solvent | 67.74 | 135.48 |
| 2 | Polysorbate 20 | 101697 | — | Emulsifier | 3.00 | 6.00 |
| 3 | Glycerol | 101976 | 36-40 | Emulsifier | 6.00 | 12.00 |
| | monooleate | | | | | |
| 4 | PEG-35 | 102491 | −4 | Surfactant | 5.00 | 10.00 |
| | castor oil | | | | | |
| 5 | Butylated | 100826 | 70 | Antioxidant | 0.20 | 0.40 |
| | hydroxytoluene | | | | | |
| 6 | Lopinavir | 102043 | 124 | API | 16.67 | 33.34 |
| 7 | Ritonavir | 102044 | 120 | API | 1.39 | 2.78 |
| | | | | synergist | | |
| | | | | TOTAL | 100.00 | 200.0 |

TABLE 14
Formulation 3n
Batch number: F139
Batch size: 200 g
Active supplier: Cipla

| | | ID | | | | |
|---|---|---|---|---|---|---|
| # | Ingredient | number | MP ° C. | Function | % w/w | g/batch |
| 1 | Oleic Acid | 101977 | 13 | Solvent | 67.74 | 135.48 |
| 2 | Polysorbate 60 | 102065 | 45-50 | Emulsifier | 3.00 | 6.00 |
| 3 | Glycerol | 101976 | 36-40 | Emulsifier | 6.00 | 12.00 |
| | monooleate | | | | | |
| 4 | PEG-35 | 102491 | −4 | Surfactant | 5.00 | 10.00 |
| | castor oil | | | | | |
| 5 | Butylated | 100826 | 70 | Antioxidant | 0.20 | 0.40 |
| | hydroxytoluene | | | | | |
| 6 | Lopinavir | 102043 | 124 | API | 16.67 | 33.34 |
| 7 | Ritonavir | 102044 | 120 | API | 1.39 | 2.78 |
| | | | | synergist | | |
| | | | | TOTAL | 100.00 | 200.0 |

TABLE 15
Formulation 3o
Batch number: F140
Batch size: 200 g
Active supplier: Cipla

| | | ID | | | | |
|---|---|---|---|---|---|---|
| # | Ingredient | number | MP ° C. | Function | % w/w | g/batch |
| 1 | Oleic Acid | 101977 | 13 | Solvent | 67.74 | 135.48 |
| 2 | Polysorbate 80 | 807339 | — | Emulsifier | 3.00 | 6.00 |
| 3 | Glycerol | 101976 | 36-40 | Emulsifier | 6.00 | 12.00 |
| | monooleate | | | | | |
| 4 | PEG-35 | 102491 | −4 | Surfactant | 5.00 | 10.00 |
| | castor oil | | | | | |
| 5 | Butylated | 100826 | 70 | Antioxidant | 0.20 | 0.40 |
| | hydroxytoluene | | | | | |
| 6 | Lopinavir | 102043 | 124 | API | 16.67 | 33.34 |
| 7 | Ritonavir | 102044 | 120 | API | 1.39 | 2.78 |
| | | | | synergist | | |
| | | | | TOTAL | 100.00 | 200.0 |

TABLE 16
Formulation 3p
Batch number: F145
Batch size: 200 g
Active supplier: Cipla

| | | ID | | | | |
|---|---|---|---|---|---|---|
| # | Ingredient | number | MP ° C. | Function | % w/w | g/batch |
| 1 | Oleic Acid | 101977 | 13 | Solvent | 63.83 | 127.67 |
| 2 | Polysorbate 20 | 101697 | — | Emulsifier | 4.30 | 8.60 |
| 3 | Glycerol | 101976 | 36-40 | Emulsifier | 5.00 | 10.0 |
| | monooleate | | | | | |
| 4 | PEG-35 | 102491 | −4 | Surfactant | 5.00 | 10.0 |
| | castor oil | | | | | |
| 5 | Butylated | 100826 | 70 | Antioxidant | 0.20 | 0.40 |
| | hydroxytoluene | | | | | |
| 6 | Lopinavir | 102043 | 124 | API | 20.00 | 40.00 |
| 7 | Ritonavir | 102044 | 120 | API | 1.67 | 3.33 |
| | | | | synergist | | |
| | | | | TOTAL | 100.00 | 200.00 |

TABLE 16A
Formulation 3p (750 mg/1500 mg capsule fills)
Batch number: F145
Active supplier: Cipla

| | | | | | | mg/ | mg/ |
|---|---|---|---|---|---|---|---|
| | | ID | | | | 750 mg | 1500 mg |
| # | Ingredient | number | MP ° C. | Function | % w/w | capsule | capsule |
| 1 | Oleic Acid | 101977 | 13 | Solvent | 63.83 | 478.7 | 957.5 |
| 2 | Polysorbate 20 | 101697 | — | Emulsifier | 4.30 | 32.3 | 64.5 |
| 3 | Glycerol | 101976 | 36-40 | Emulsifier | 5.00 | 37.5 | 75.0 |
| | monooleate | | | | | | |
| 4 | PEG-35 | 102491 | −4 | Surfactant | 5.00 | 37.5 | 75.0 |
| | castor oil | | | | | | |
| 5 | Butylated | 100826 | 70 | Antioxidant | 0.20 | 1.5 | 3.0 |
| | hydroxytoluene | | | | | | |
| 6 | Lopinavir | 102043 | 124 | API | 20.00 | 150.0 | 300.0 |
| 7 | Ritonavir | 102044 | 120 | API | 1.67 | 12.5 | 25.0 |
| | | | | synergist | | | |
| | | | | TOTAL | 100.00 | 750.0 | 1500.0 |

TABLE 17
Comparative Formulation 3q
Batch number: F143
Batch size: 100 g
Active supplier: Cipla

| | | ID | | | | |
|---|---|---|---|---|---|---|
| # | Ingredient | number | MP ° C. | Function | % w/w | g/batch |
| 1 | Oleic Acid | 101977 | 13 | Solvent | 51.74 | 51.74 |
| 2 | Polysorbate 20 | 101697 | — | Emulsifier | 19.00 | 19.00 |
| 3 | Glycerol | 101976 | 36-40 | Emulsifier | 6.00 | 6.00 |
| | monooleate | | | | | |
| 4 | PEG-35 | 102491 | −4 | Surfactant | 5.00 | 5.00 |
| | castor oil | | | | | |
| 5 | Butylated | 100826 | 70 | Antioxidant | 0.20 | 0.20 |
| | hydroxytoluene | | | | | |
| 6 | Lopinavir | 102043 | 124 | API | 16.67 | 16.67 |
| 7 | Ritonavir | 102044 | 120 | API | 1.39 | 1.39 |
| | | | | synergist | | |
| | | | | TOTAL | 100.00 | 100.0 |

TABLE 18
Formulation 3r
Batch number: F146
Batch size: 200 g
Active supplier: Cipla

| | | ID | | | | |
|---|---|---|---|---|---|---|
| # | Ingredient | number | MP ° C. | Function | % w/w | g/batch |
| 1 | Oleic Acid | 101977 | 13 | Solvent | 68.83 | 137.67 |
| 2 | Polysorbate 20 | 101697 | — | Emulsifier | 4.30 | 8.60 |
| 3 | Glycerol | 101976 | 36-40 | Emulsifier | 5.00 | 10.00 |
| | monooleate | | | | | |
| 4 | Butylated | 100826 | 70 | Antioxidant | 0.20 | 0.40 |
| | hydroxytoluene | | | | | |
| 5 | Lopinavir | 102043 | 124 | API | 20.00 | 40.00 |
| 6 | Ritonavir | 102044 | 120 | API | 1.67 | 3.33 |
| | | | | synergist | | |
| | | | | TOTAL | 100.00 | 200.0 |

TABLE 19
Formulation 3s
Batch number: F147
Batch size: 100 g
Active supplier: Cipla

| | | ID | | | | |
|---|---|---|---|---|---|---|
| # | Ingredient | number | MP ° C. | Function | % w/w | g/batch |
| 1 | Oleic Acid | 101977 | 13 | Solvent | 52.25 | 52.25 |
| 2 | Polysorbate 20 | 101697 | — | Emulsifier | 18.49 | 18.49 |
| 3 | Glycerol | 101976 | 36-40 | Emulsifier | 6.00 | 6.00 |
| | monooleate | | | | | |
| 4 | PEG-35 | 102491 | −4 | Surfactant | 5.00 | 5.00 |
| | castor oil | | | | | |
| 5 | Butylated | 100826 | 70 | Antioxidant | 0.20 | 0.20 |
| | hydroxytoluene | | | | | |
| 6 | Lopinavir | 102043 | 124 | API | 16.67 | 16.67 |
| 7 | Ritonavir | 102044 | 120 | API | 1.39 | 1.39 |
| | | | | synergist | | |
| | | | | TOTAL | 100.00 | 100.0 |

TABLE 20
Formulation 3t
Batch number: F149
Batch size: 100 g
Active supplier: Cipla

| | | ID | | | | |
|---|---|---|---|---|---|---|
| # | Ingredient | number | MP ° C. | Function | % w/w | g/batch |
| 1 | Oleic Acid | 101977 | 13 | Solvent | 48.64 | 48.64 |
| 2 | Polysorbate 20 | 101697 | — | Emulsifier | 11.36 | 11.36 |
| 3 | Glycerol | 101976 | 36-40 | Emulsifier | 13.64 | 13.64 |
| | monooleate | | | | | |
| 4 | PEG-35 | 102491 | −4 | Surfactant | 4.49 | 4.49 |
| | castor oil | | | | | |
| 5 | Butylated | 100826 | 70 | Antioxidant | 0.20 | 0.20 |
| | hydroxytoluene | | | | | |
| 6 | Lopinavir | 102043 | 124 | API | 20.00 | 20.00 |
| 7 | Ritonavir | 102044 | 120 | API | 1.67 | 1.67 |
| | | | | synergist | | |
| | | | | TOTAL | 100.0 | 100.0 |

### Example 4—Comparative Formulation 1 (Ointment)

An oleic acid/stearic acid ointment formulation containing lopinavir and ritonavir was prepared as follows, in accordance with the components specified in Table 21:
i. Into a mixer was added the following materials—3, 4, 5, 6, 7, 8, 9, 1, 10 & 11.
ii. Air was excluded from the interior of the vessel.
iii. The mixture was heated to 70° C. with low shear mixing, to achieve a clear, transparent melt.
iv. To the mixer was added the following material—2.
v. Air was excluded from the interior of the vessel.
vi. The batch was mixed via low shear, to finely disperse the HPMC (2) within the melt.
vii. The batch temperature was reduced to 45° C. while maintaining low shear mixing.
viii. The product was discharged to a storage vessel and air was excluded during storage.
ix. As required, the formulation (800 mg) was then filled into hard gelatin capsules and sealed with ethanol.

TABLE 21

| | | ID | | | | |
|---|---|---|---|---|---|---|
| # | Ingredient | number | MP ° C. | Function | % w/w | kg/batch |
| 1 | Oleic acid** | 101977 | 13 | Solvent | 62.823 | 12.565 |
| 2 | Hypromellose 2208 | 101823 | >150 | Muco- | 1.00 | 0.200 |
| | (4000cps) | | | adhesive | | |
| 3 | Mono di glycerides | 100279 | 58-59 | Thickener | 5.00 | 1.000 |
| | (type 1) | | | | | |
| 4 | White ceresin wax | 101821 | ~65 | Thickener | 6.00 | 1.200 |
| 5 | Hydrogenated | 101478 | ~60 | Thickener | 10.00 | 2.000 |
| | vegetable oil (type 1) | | | | | |
| 6 | Polyoxyl 100 stearate | 102002 | 54-59 | Blending | 2.00 | 0.400 |
| | | | | agent | | |
| 7 | Stearic acid | 101714 | 69 | Stiffening | 4.50 | 0.900 |
| | | | | agent | | |
| 8 | Glycerol monooleate* | 101976 | 36-40 | Blending | 3.00 | 0.600 |
| | | | | agent | | |
| 9 | Butylated | 100826 | 70 | Antioxidant | 0.20 | 0.04000 |
| | hydroxytoluene | | | | | |
| 10 | Lopinavir** | 102043 | 124 | API | 5.000 | 1.000 |
| 11 | Ritonavir*** | 102044 | 120 | API | 0.4775 | 0.0955 |
| | | | | synergist | | |
| | | | | TOTAL | 100.00 | 20.000 |

*Melt completely/mix source supply prior to dispensing
**N₂ purge source supply after sampling
***Protect from UV light

The ointment formed according to Example 4 has a complex viscosity of 1500-2000 cP.s. when the complex viscosity is measured at an angular frequency of 0.1 rad/s. Complex viscosity was measured on a Discovery Hybrid Rheometer (Model HR-3, TA instruments) [the sample was introduced onto the peltier plate (base; 37° C.) in excess (1-2 g), and the spindle lowered to make contact with the sample. The excess sample was cleaned up. The spindle was then rotated in a predetermined fashion to exert a series of shear forces on the sample. The oscillation frequency method parameters were speed range 0.1 to 1 rad/s; strain 0.5%; spindle 40 mm parallel plate; gap setting 1000 μm].

### Example 5—Comparative Formulation 2

WO2002/096395 discloses soft elastic capsules filled with a formulation comprising oleic acid, lopinavir and ritonavir. A comparative formulation was formed according to Fill Batch PS-A (WO2002/096395; page 24) as follows, containing the component amounts specified in Table 22:
i. Into a mixer was added oleic acid.
ii. Propylene glycol was added to the vessel and mixed for 5 mins.
iii. Ritonavir was added to the vessel and stirring was continued until dissolution had occurred.
iv. Lopinavir was added to the vessel and stirring was continued until dissolution had occurred.
v. Cremophor® EL was added and mixing was continued for 10 minutes.
vi. The product was stored under nitrogen atmosphere.
vii. 800 mg samples of Comparative Formulation 2 were then filled into hard gelatin capsules and sealed with ethanol.

TABLE 22

| | | ID | | | | |
|---|---|---|---|---|---|---|
| # | Ingredient | number | MP ° C. | Function | % w/w | mg/batch |
| 1 | Oleic acid | 101977 | 13 | Solvent | 68.6 | 603 |
| 5 | Propylene Glycol | 101180 | −59 | Cosolvent | 10.1 | 89 |
| 6 | Cremophor ® EL | Lot | −4 | Surfactant | 2.4 | 21 |
| | | 85728947G0 | | | | |
| 10 | Lopinavir** | 102043 | 124 | API | 15.1 | 133 |
| 11 | Ritonavir*** | 102044 | 120 | API | 3.8 | 33 |
| | | | | synergist | | |
| | | | | TOTAL | 100.0 | 879 |

**N₂ purge source supply after sampling
***Protect from UV light

### Example 6: Stability Studies

The compositions according to Example 3 have been proven to be stable during the process employed to manufacture them (i.e. processes described in Example 3). Additionally, the compositions according to Example 3 have been shown to be stable upon storage.

Formulations 3a, 3b and 3c were stored for 1 month at 5° C. After this time, they released API to completion in dissolution testing according to Example 7, indicating that the API loading has not been degraded during storage.

The following analytical methodology was used to analyse the formulations.

### UHPLC:

### Equipment Parameters:

| System Description | Waters Acquity H-Class UHPLC with a UV-VWD or UV-PDA | |
|---|---|---|
| | detector (or equivalent) | |
| Column | Acquity BEH C8, 2.1 mm × 50 mm, 1.7 μm (or equivalent) | |
| Column Temperature | 50° C. | |
| Detector Wavelength | Ritonavir: 240 nm | |
| | Lopinavir: 215 nm | |
| | PDA: 200-300 nm | |
| Flow Rate | 0.6 mL/min | |
| Injection Volume | 2 μL | |
| Sampling Rate | 5 points/sec | |
| Inject Wash | Post-inject: 6 seconds | |
| Integration | Peak area | |
| Run Time | About 17 minutes | |
| Retention Time | Ritonavir: about 5.3 minutes (240 nm) | |
| | Lopinavir: about 6.3 minutes (215 nm) | |
| Mobile phase A | Aqueous component of | 30 mM KH₂PO₄, for example to |
| | mobile phase A | prepare 1 L of 30 mM KH₂PO₄ |
| | | dissolve 4.1 g of potassium |
| | | dihydrogen phosphate in 1000 mL of |
| | | purified water. Pass through a 0.2 μm |
| | | membrane filter (e.g. Millipore 0.2 |
| | | μm GNWP) |
| | Organic component of | A 35:25:20 mixture of |
| | mobile phase A | acetonitrile:tetrahydrofuran:methanol |
| | Mobile phase A | 35:65 mixture of organic |
| | | component:aqueous component. |
| | | Degas by sonication. Do not filter |
| | | under vacuum after addition of |
| | | organic component. Retention time of |
| | | analytes are extremely sensitive to |
| | | mobile phase composition. |

| | |
|---|---|
| Mobile phase B | Acetonitrile. Degas by sonication |
| Diluent | 50:50 mixture of acetonitrile:aqueous component of mobile |
| | phase A |
| Sample preparation | All solutions must be freshly prepared on the day of analysis. |
| | Step 1: Weight about 1250 mg of formulation into a 50 mL |
| | volumetric flask. Add 2.5 mL of 1-butanol and about 10 ml of |
| | acetonitrile and shake vigorously to disperse the formulation. |
| | Using a transfer pipette, rinse any sample from the interior walls |
| | and neck of the flask with about 10 mL more of acetonitrile. |
| | Sonicate the mixture for 10 minutes with occasional swirling to |
| | completely disperse the sample. Allow to equilibrate to room |
| | temperature and dilute to volume with acetonitrile. Mix well by |
| | inversion. |
| | Step 2: Let the mixture from step 1 stand to allow any solid |
| | particles settle at the bottom of the flask. Carefully pipette 5.0 |
| | mL of the clear supernatant liquid into a volumetric flask (volume |
| | of flask picked so final concentration of lopinavir and ritonavir |
| | are suitable for detection by UHPLC, for example final |
| | concentration of 300 μg/mL Lopinavir and 25 μg/mL Ritonavir |
| | for a 12:1 (by weight) Lopinavir:Ritonavir formulation). Dilute to |
| | volume with dilute and mix well by inversion. Chill the sample in |
| | the fridge for at least 30 minutes. When cold, filter the sample |
| | solution through a syringe filter (with a 4 mL discard volume, |
| | polypropylene housing and a 0.2 μm regenerated cellulose filter |
| | membrane (e.g. 0.2 μm Phenex-RC)) directly into an HPLC vial, |
| | discarding the first 4 mL of filtrate. |

| | Mobile phase | Mobile phase |
|---|---|---|
| Time (min) | A (% v/v) | B (% v/v) |
| 0 | 100 | 0 |
| 8.5 | 100 | 0 |
| 9 | 60 | 40 |
| 12 | 60 | 40 |
| 12.5 | 100 | 0 |
| 17 | 100 | 0 |

### Example 7: API Dissolution Performance

### Example 7A—0.7% SLS Dissolution Media

Encapsulated compositions of the present invention prepared according to Example 3 were subjected to dissolution studies in 0.7% sodium laureth sulfate (SLS) media at 37±0.5° C., carried out in USP II apparatus at 25 rpm for 0 to 60 min and then at 200 rpm 60 to 75 min (hard gel capsules with sinkers). All analyses were carried out in duplicate.

FIG. 1 shows the results of the dissolution studies for Formulations 3a, 3b, and 3c. All three formulations exhibited 40-60% lopinavir dissolution after 10 mins, 70-90% lopinavir dissolution after 45 mins and 90-100% lopinavir dissolution after 75 mins.

The encapsulated composition prepared according to Example 3 having 4% wt polyoxyl 100 stearate and 6% wt glycerol monooleate (Formulation 3a) was compared with encapsulated compositions prepared according to Example 3 having 3% wt glycerol monooleate and 10% wt glycerol monooleate (Comparative Formulations 3f and 3h respectively). Dissolution testing was carried out according to the methodology described above. All analyses were carried out in duplicate.

FIGS. 2 and 3 show the results of these comparative dissolution studies in terms of the release of lopinavir and ritonavir respectively. It can be seen that when both the emulsifiers are present at a total emulsifier level of 10% wt, the in vitro release for lopinavir is faster than when polyoxyl 100 stearate is excluded, regardless of whether the remaining glycerol monooleate emulsifier content is at a lower level (3% wt—Ex. 3f), or is increased to 10% wt (Ex. 3h).

The encapsulated composition prepared according to Example 3 having 10% wt glycerol monooleate (Comparative Formulation 3h) was compared with encapsulated compositions prepared according to Example 3 having 2% wt polyoxyl 100 stearate and 10% wt polyoxyl 100 stearate (Comparative Formulations 3e and 3 g respectively). Dissolution testing was carried out according to the methodology described above. All analyses were carried out in duplicate.

FIGS. 4 and 5 show the results of these comparative dissolution studies in terms of the release of lopinavir and ritonavir respectively. Although there is some variability between replicates for the comparative formulations comprising only polyoxyl 100 stearate as emulsifier, it can be seen that even the fastest lopinavir release for a polyoxyl 100 stearate-only formulation (Comparative Formulation 3e; 77% release at 45 mins) is inferior to the two-emulsifier system release (c.f. FIG. 2: Formulation 3a; 93% release at 45 mins). Therefore, when both emulsifiers are present at a total emulsifier level of 10% wt, the in vitro release for lopinavir is faster than when glycerol monooleate is excluded, regardless of whether the remaining polyoxyl 100 stearate emulsifier content is at a lower level (2% wt—Ex. 3e), or is increased to 10% wt (Ex. 3 g).

FIG. 6 also shows the comparison of the lopinavir dissolution profiles for formulation 3a (4% wt polyoxyl 100 stearate and 6% wt glycerol monooleate), comparative formulation 3 g (10% wt polyoxyl 100 stearate) and comparative formulation 3h (10% wt glycerol monooleate). Formulation 3a demonstrated 50-60% lopinavir dissolution after 10 mins and approx. 90% lopinavir dissolution after 45 mins; comparative formulation 3 g demonstrated approx. 10% lopinavir dissolution after 10 mins and 45-55% lopinavir dissolution after 45 mins; comparative formulation 3h demonstrated 30-40% lopinavir dissolution after 10 mins and 60-70% lopinavir dissolution after 45 mins. While the comparative formulation containing only 10% wt glycerol monooleate emulsifier gave superior dissolution to the comparative formulation containing only 10% wt polyoxyl 100 stearate emulsifier, both systems were markedly inferior to the formulation having 10% wt emulsifier made up from the combination of the two emulsifiers.

The encapsulated composition prepared according to Example 3 having 4% wt polyoxyl 100 stearate and 6% wt glycerol monooleate (Formulation 3a) was compared with that of encapsulated compositions prepared according to Examples 4 and 5 (Comparative Formulations 1 and 2 respectively) according to the dissolution testing methodology described above. All analyses were carried out in duplicate.

FIG. 7 shows the results of the comparative lopinavir dissolution studies. Formulation 3a exhibited 50-60% lopinavir dissolution after 10 mins, approx. 90% lopinavir dissolution after 45 mins and approx. 100% lopinavir dissolution after 75 mins. The soft gel Comparative Formulation 2 (Example 5) exhibited 10-20% lopinavir dissolution after 10 mins, 40-60% lopinavir dissolution after 45 mins and 90-100% lopinavir dissolution after 75 mins. The ointment Comparative Formulation 1 (Example 4) exhibited 0% API dissolution up to 60 mins and 0-10% lopinavir dissolution after 75 mins.

It can be seen therefore that the formulations according to the present invention have a faster and more complete API dissolution profile in the SLS media than the comparative formulations tested.

Further encapsulated compositions of the present invention prepared according to Example 3 were subjected to dissolution studies in 0.7% sodium laureth sulfate (SLS) media at 37±0.5° C., carried out in USP II apparatus at 50 rpm for 0 to 60 min (hard gel capsules with sinkers).

The encapsulated composition prepared according to Example 3 having 4% wt polyoxyl 100 stearate, 6% wt glycerol monooleate and 5% wt PEG 35 castor oil (Formulation 3j) was compared with encapsulated compositions prepared according to Example 3 having 6% wt glycerol monooleate and 5% wt PEG 35 castor oil (comparative Formulation 3k), 4% wt polyoxyl 100 stearate and 5% wt PEG 35 castor oil (comparative Formulation 31) and a composition prepared according to Example 5 (Comparative Formulation 2) according to the dissolution testing methodology described above. FIG. 8A shows that Formulation 3j gave more rapid dissolution than Example 5 and that removing the glycerol monooleate from this formulation gave an inferior API release (comparative Formulation 31). Removal of the PEG 100 stearate from the formulation actually gave in this case an increased release rate (comparative Formulation 3k). FIG. 8B shows that when polysorbate 20 (Formulation 3m), polysorbate 60 (Formulation 3n), or polysorbate 80 (Formulation 3o) was used as the high HLB emulsifier, all three formulations have a similar lopinavir release and all are superior to soft gel Comparative Formulation 2 (Example 5) in SLS media.

### Example 7B—0.7% CTAB Dissolution Media

Encapsulated compositions of the present invention prepared according to Example 3 were subjected to dissolution studies in 0.7% cetyltrimethylammonium bromide (CTAB) media at 37 0.5° C., carried out in USP II apparatus at 50 rpm for 0 to 60 min (hard gel capsules with sinkers).

FIG. 9A shows that in the CTAB media Formulation 3a gave lower release than Comparative Formulation 2 (Example 5). Addition of PEG 35 castor oil to the formulation (Formulations 3i and 3j) improved the API release and addition of 5% wt PEG 35 castor oil (Formulation 3j) gave superior release to Comparative Formulation 2 (Example 5). FIG. 9B shows that removing the glycerol monooleate from Formulation 3j gave a faster API release rate in CTAB media (comparative Formulation 31). Removal of the PEG 100 stearate from the formulation did not affect the release rate (comparative Formulation 3k).

FIG. 10 shows that when polysorbate 20 (Formulation 3m), polysorbate 60 (Formulation 3n), or polysorbate 80 (Formulation 3o) was used as the high HLB emulsifier, all three formulations have a similar lopinavir release and all are greatly superior to soft gel Comparative Formulation 2 (Example 5) in CTAB media.

### Example 7C—0.05 M Brij® 10 Dissolution Media

Encapsulated compositions of the present invention prepared according to Example 3 were subjected to dissolution studies in 0.05 M PEG 10 oleyl ether (Brij® 10) with 10 mM sodium monobasic phosphate pH 6.8 media at 37±0.5° C., carried out in USP II apparatus at 50 rpm for 0 to 60 min (hard gel capsules with sinkers).

FIG. 11 shows that in the Brij® 10 media Formulation 3a gave lower release than Comparative Formulation 2 (Example 5). Removal of the PEG 100 stearate (comparative Formulation 3h) was found to give a slower API release rate. FIG. 12 shows that addition of PEG 35 castor oil to the formulation (Formulations 3i and 3j) improved the API release and addition of 5% wt PEG 35 castor oil (Formulation 3j) gave comparable release to Comparative Formulation 2 (Example 5) in this media.

FIG. 13 shows that when polysorbate 20 (Formulation 3m), polysorbate 60 (Formulation 3n), or polysorbate 80 (Formulation 3o) was used as the high HLB emulsifier, all three formulations have a similar lopinavir release and all are comparable to soft gel Comparative Formulation 2 (Example 5) in Brij® 10 media.

Table 23 summarises the lopinavir release data of all the formulations tested in the Brij® dissolution media (0.05 M PEG 10 oleyl ether (Brij® 10) with 10 mM sodium monobasic phosphate pH 6.8 media at 37±0.5° C., carried out in USP 11 apparatus at 50 rpm for 0 to 60 min).

TABLE 23
lopinavir dissolution in 0.05M Brij ® 10 dissolution media

| | % Dissolution release | | | | |
|---|---|---|---|---|---|
| | Time (min) | | | | |
| Formulation | 10 | 20 | 30 | 45 | 60 |
| Formulation 3a | 14 | 22 | 31 | 46 | 50 |
| Comparative | 4 | 8 | 18 | 29 | 41 |
| Formulation 3h | | | | | |
| Formulation 3i | 55 | 63 | 73 | 81 | 87 |
| Formulation 3j | 82 | 90 | 93 | 96 | 97 |
| Comparative | 83 | 91 | 94 | 96 | 96 |
| Formulation 3k | | | | | |
| Comparative | 90 | 95 | 96 | 98 | 98 |
| Formulation 3l | | | | | |
| Formulation 3m | 81 | 92 | 93 | 96 | 96 |
| Formulation 3n | 76 | 91 | 94 | 95 | 96 |
| Formulation 3o | 74 | 93 | 93 | 94 | 95 |
| Comp. Formulation | 85 | 89 | 92 | 93 | 94 |
| 2 (Ex. 5) | | | | | |

### Example 7D—Simulated Vaginal Fluid (SVF) Dissolution Media

Encapsulated compositions of the present invention prepared according to Example 3 were subjected to dissolution studies in pH 4.2 SVF media at 37±0.5° C., carried out in USP II apparatus at 100 rpm for 0 to 480 min (hard gel capsules with sinkers). SVF media was an aqueous solution of sodium chloride (3.51 g/L), potassium hydroxide (1.40 g/L), calcium hydroxide (0.222 g/L), bovine serum albumin (0.018 g/L), lactic acid (2.00 g/L), acetic acid (1.0 g/L), glycerol (0.16 g/L), urea (0.40 g/L) and glucose (5.00 g/L).

The data in Table 24 shows that in the SVF media Formulations 3m and 3p and Comparative Formulation 2 (Example 5) gave minimal lopinavir release. Increasing the total emulsifier content to 30% (comparative formulation 3q) gave increased lopinavir release in non-surfactant SVF media.

TABLE 24
lopinavir dissolution in SVF media

| | % Dissolution release | | | | | |
|---|---|---|---|---|---|---|
| | Time (min) | | | | | |
| Formulation | 30 | 60 | 120 | 240 | 360 | 480 |
| Comp. Formulation 2 | 1 | 1 | 2 | 2 | 2 | 2 |
| (Ex. 5) | | | | | | |
| Formulation 3m | 2 | 2 | 2 | 2 | 2 | 2 |
| Formulation 3p | 2 | 3 | 2 | 2 | 2 | 2 |
| Comp. Formulation 3q | 13 | 13 | 14 | 15 | 14 | 14 |

### Example 8: Emulsion Droplet Size Testing

Formulations 3a, 3b and 3c and comparative formulations 2, 3f and 3h were analysed for emulsion droplet size as follows.

1 g of each sample was dispersed in 10 mL milli-Q water. The dispersed sample was followed by vortex on high speed for 1 minute at room temperature, before being added to a cuvette. The cuvette was placed in a Zetasizer (Zetasizer Nano series, Malvern Instrument, UK; measuring range 1 nm to 6000 μm) and analysed by dynamic light scattering (particle refractive index: 1.30; absorption: 0.01; dispersant refractive index: 1.33). Each sample was measured over 3 cycles and the average and standard deviation measurement was obtained by Excel® software. The results are shown in Table 25.

TABLE 25

| | | Total | Average |
|---|---|---|---|
| | | emulsifier | Droplet size ± |
| Formulation | Emulsifier levels (% wt) | content (% wt) | s.d. (nm) |
| 3a | 4% polyoxyl 100 stearate, 6% | 10% | 409 ± 42 |
| | glycerol monooleate | | |
| 3a (repeat) | 4% polyoxyl 100 stearate, 6% | 10% | 236 ± 11 |
| | glycerol monooleate | | |
| 3b | 2% polyoxyl 100 stearate, 3% | 5% | 718 ± 66 |
| | glycerol monooleate | | |
| 3c | 1% polyoxyl 100 stearate, 1.5% | 2.5% | 1124 ± 144 |
| | glycerol monooleate | | |
| Comparative | 2.4% polyoxyl ethylene 35 | 2.4% | 795 ± 95 |
| formulation 2 | castor oil | | |
| Comparative | 3% glycerol monooleate | 3% | 5675 ± 536 |
| formulation 3f | | | |
| Comparative | 10% glycerol monooleate | 10% | 4764 ± 459 |
| formulation 3h | | | |

The ability of the formulations 3a-3c to yield a fine emulsion upon contact with water confirms that they are self-emulsifying compositions. The droplet size results for the formulations 3a-3c relate to the total emulsifier level; the higher the emulsifier content, the smaller the droplet size. Formulations 3a and 3b exhibited superior emulsification compared to comparative formulation 2, as evidenced by the smaller emulsion droplet sizes.

From the dissolution studies described in Example 7A, comparative formulations with the best performing single-emulsifier (glycerol monooleate; FIG. 6) were compared with the formulations of the invention. It can be seen from Table 11 that the droplet sizes for the two-emulsifier formulations (e.g. formulations 3a and 3b) is an order of magnitude smaller than the single-emulsifier formulations (comparative formulations 3f and 3h), regardless of whether the single-emulsifier equates to an ‘individual’ or ‘total’ glycerol monooleate level compared to the two-emulsifier formulations. This indicates that both the emulsifiers (high HLB and low HLB) present in the formulations according to the present invention are necessary to yield a reduced emulsion droplet size.

### Example 9: A Phase 1, Single Centre, Double Blind, Randomised, Parallel Group, Ascending Single and Multiple Dose, Safety and Tolerability, Pharmacokinetic (PK) and Pharmacodynamic (PD) Study of Formulations 3D in Healthy Women Volunteers

The composition according to Example 3 (Formulations 3p) is investigated according to the clinical trial described below.

### Study Objectives

1. To evaluate the safety, PK and PD of compositions in healthy women volunteers after multiple doses of Formulation 3p.
2. To observe the rates of side effects reported by women using the compositions compared to placebo.

### Investigational Plan/Study Design

This study comprises 6 healthy volunteers with no cervical pathology. Of the participants, 3 receive active and 3 receive placebo (identical formulation to formulation 3p but absent lopinavir or ritonavir). For all formulations tested, the amount of the composition administered per dose is 1500 mg. For Formulation 3p this equates to 300 mg of lopinavir and 25 mg of ritonavir being administered to the patients per dose.
Period 1: Single dose of Formulation 3p or placebo formulation followed by confinement. PK blood sampling during confinement.
Period 2: 21 daily doses of Formulation 3p or placebo formulation followed by PK blood sampling.

### Participation Criteria

### Inclusion Criteria:

a. Women, 20 to 45 years old, with an intact uterus and vagina.
b. Generally, in good health with no clinically significant pulmonary, cardiac, gastroenterological, pancreatic, neurologic, renal, musculoskeletal, rheumatologic, metabolic, neoplastic, or endocrine disease.
c. BMI of =>19 and <=30.0
d. ECG and vital signs within normal ranges
e. Agree to no Alcohol from 48 hours prior to dosing in period 1 until 7 days after receiving the final dose in period 2.
f. Abstain from food or beverages containing grapefruit, starfruit, pomegranate, pineapple, or pomelo for the entire study
g. Able and willing to abstain from sexual intercourse+/−6 hours around dosing within Periods 1 and 2
h. Able and willing to use stringent methods of contraception after required abstinence period through to Day 29 (7 days after receiving the final dose in period 2), including the use of a non-latex condom (for partner protection) and a second acceptable contraception method; vasectomy, contraceptive pill, contraceptive implants or IUDs are allowed. (note: IUDs should have been inserted at least 1 month prior to enrolment and not because of the involvement in this study)
i. Agree to abstain from activities such as vaginal douching or insertion of any vaginal products other than the study drug for at least 48 hours prior to enrolment and throughout the study.
j. Negative Pap test at screening or within 3 years of enrolment and no history of cervical intraepithelial lesions within the previous 3 years
k. Able and willing to return to the clinic for all study procedures.
l. Able and willing to provide informed consent.

### Exclusion Criteria:

a. Women who are pregnant, plan to become pregnant in the next 3 months, or lactating females.
b. History of genital herpes with >3 outbreaks per year, or active non-HPV vaginal infection
c. Positive result for Hep B, Hep C or HIV.
d. Have an active pelvic infection (positive urine screen for gonorrhoea or chlamydial infection, positive test and symptoms for bacterial vaginosis, candida vaginitis or trichomonal vaginitis)
e. Current or recent abnormal vaginal discharge and/or abnormal vaginal bleeding, within the 3 months prior to randomization as accessed by Investigator.
f. Had an abortion or miscarriage within the 3 months prior to randomization
g. Currently taking any of the following medications: oral corticosteroids, inhaled salmeterol and fluticasone; immunomodulatory treatments, over the counter (OTC) intra-vaginal preparation, or any prescription that in the opinion of the Investigator could interfere with the interpretation of the results.
h. Currently taking any of the medications listed here—Alfuzosin, Amiodarone, dronedarone, Ranolazine, Fusidic Acid, Colchicine, Astemizole, terfenadine, Lurasidone, Pimozide, Quetiapine, Dihydroergotamine, ergonovine, ergotamine, methylergonovine, Cisapride, Lovastatin, simvastatin, Avanafil, Sildenafil, Vardenafil, Oral midazolam, triazolam, St. John's wort.
i. Recent history (within previous 3 months) of Stevens-Johnson syndrome, erythema multiforme, urticaria, angioedema, deep vein thrombosis, tinnitus, vertigo, blood glucose disorders, pancreatitis, haemophilia.
j. Hypersensitivity to any component of Example 3 vaginal formulation excipients
k. Participation in any clinical study with an experimental medication or device within 30 days or 5 half-lives (whichever is longer) of enrolment.
l. Current alcohol or substance abuse as assessed by the Investigator.
m. An employee or first-degree family member of an employee, the Sponsor, the CRO or study site.
n. Not having a GP

### Screening Evaluations:

The screening evaluations must have been made within 3 months of randomization into the study. Screening consists of the following components:

### Demographic/Medical History

A complete medical history is taken from each participant.

### Physical Examination

The physical examination consists of a review of body systems with height and weight (in indoor clothing).

### Blood Tests

The following laboratory blood tests are performed:
Electrolytes (sodium and potassium), ALT, GGT, ALP, albumin, total protein, total bilirubin, urea, uric acid, serum creatinine, TFT, fasting lipids, amylase, glucose, and HbA1c
Haemoglobin, red cell count, PCV, MCV, MCH, platelet count, white cell count, neutrophils, lymphocytes, monocytes, eosinophils and basophils. CD4/CD8 counts
HIV and hepatitis B and C.

The measurements at screening serve as a baseline to monitor any abnormalities that may manifest as a result of dosing

### Other Tests

Drugs of abuse testing are carried out on all participants as part of the screening procedures. A urine sample is required to test for cannabinoids (marijuana), amphetamines, benzodiazepines and opiates (i.e. morphine, heroin and codeine). Urinalysis dipstick to check for protein, leucocytes, nitrites, pH, specific gravity, glucose, ketones, and blood. Vaginal swabs for microbiology (gonorrhoea, Chlamydia, bacterial vaginosis, candida) and HPV genotyping. Alcohol breath testing is carried out at the Clinical Site on the first night of each confinement period. Serum HCG testing is carried out on all participants as part of the screening procedures and within 3 days before the 1st dose.

### Vital Signs

Vital signs are recorded and consist of blood pressure (supine and sitting), heart rate, temperature and respiratory rate. Participants' vital signs should be within the following limits: Heart rate ≥60 or ≤99 beats/minute

### Supine:

Systolic Blood Pressure ≥90 or ≤160 mm Hg; Diastolic Blood Pressure ≥50 or ≤90 mm Hg

### Sitting:

Systolic Blood Pressure ≥90 or ≤160 mm Hg; Diastolic Blood Pressure ≥50 or ≤90 mm Hg
Temperature ≥36° C. or ≤37.7° C.
Respiratory Rate ≥12 or ≤20 breaths/minute

### Summary of Study Activities/Schedule of Events

Informed consent is needed from each participant. Participants are screened to confirm study eligibility.

Randomisation Participants are randomized following the Principal Investigator or their delegates documented acceptance of participants following review of completed screening procedures.

### Study Confinement

Participants arrive at approximately 5 μm on Day 1 and Day 22. The duration of study confinement is approximately 27 hours. Participants are released from the clinical site once the 24-hour post dose assessments has been completed.

### Dosing

Dosing begins at approximately 8 μm on each day dosing is scheduled. Participants are instructed to insert the medication in private.

### Sample Collection

Vaginal swabs are self-administered by the participants. PK Blood samples: blood samples (8 mL) are drawn through venous catheters and transferred into vacutainers containing sodium heparin as the anti-coagulant. The time of collection is recorded as the time the full 8 mL of blood is collected. The venous catheters are kept patent by flushing with 1.5 mL-2.0 mL of heparinized saline following each sample (0-24 hours). The sampling intervals are at: Day 1-2: 0, 1, 2, 4, 8, 12, 24 hours; Day 22-23: 0, 1, 2, 4, 8, 12, 24 hours. Samples are collected at their due time. Any deviation is noted.

### Sample Processing and Storage

Plasma: Plasma is separated by centrifugation at 3500 rpm for 5 minutes at about 4° C. No aids for separation of plasma from red cells are used. The plasma sample is transferred with clean pipettes. The assay is determined using a validated Analytical method. Each plasma sample is placed into a polypropylene storage tube with a screw cap. The plasma is stored frozen at −60° C. or colder at the clinical site pending transfer to a Laboratory for assay.

### End of Study

Within one week after the last study day, each participant is required to provide a blood sample for analysis. Any abnormalities as compared to initial screening are monitored and followed up until they return to normal. Participants are assessed for the occurrence of adverse events from consent until the last study day in each cohort. Vital signs (blood pressure, heart rate, respiratory rate and temperature) at last study visit.
Laboratory tests (haematology (CBC, CD4+/CD8+ peripheral lymphocyte count, biochemistry (RFT, LFT, electrolytes, TFT, fasting lipids, HbA1c, amylase), Serum HCG Pregnancy and urinalysis (dipstick), at last study visit.
A follow-up phone call to each participant is made within 7 days (+2 days) of the end of the study to record any possible Adverse Events (AE) post study. Any events are recorded in source documentation.

All AE's are followed-up until resolution, or until the Investigator was of the opinion that follow-up is no longer required, or until 30 days from the last dose (as long as the Investigator is satisfied that follow-up is no longer required), whichever is earlier.

### Adverse Events

During confinement the designated Supervisor for the study or a delegated representative must be present at the study site throughout the study. Principal Investigator or at least one delegated Trial Physician is on call throughout the studies. On all study visits each participant is asked how they felt. This occurs at each sampling point throughout the study. AE's are recorded in source documentation. Each AE is classified by the Principal Investigator as a serious adverse event (SAE) or non-serious. Non-serious adverse events are assessed as being mild, moderate, or severe to describe the maximum intensity of the AE. The Principal Investigator also provides the possible relationship between the AE and the study medication as highly probable, probable, possible, remotely or not (“no”) related to the study medication. The Principal Investigator state if the cause of the AE is related to the concurrent non-investigational medication(s) if any are being taken, an underlying disease, a combination of these factors or is unknown.

### Pharmacokinetic Analyses:

### Pharmacokinetic Parameters:

The area under the plasma drug concentration time curve (AUC), the peak plasma drug concentration (Cmax) and the time to maximum drug concentration (Tmax) are determined for lopinavir and ritonavir for each subject receiving active treatment.

The plasma drug concentration (C) versus the real sampling time (t) data are analysed by a “non-compartmental” method to obtain the pharmacokinetic parameters. Initially the plasma data in the post distribution phase of the plasma concentration—time plot are fitted using linear regression to:

In C=In Co−t.Kel

where Co is the zero-time intercept of the extrapolated terminal phase and Kel is the terminal elimination rate constant. The area (AUC₀₋ₜ) from time zero to the last determined concentration-time point (t) in the post distribution phase is calculated using the trapezoidal rule.

### Cmax

For the ointment comparative formulation 1 (Example 4) containing 12% w/w lopinavir, following a topical dose of 300 mg lopinavir daily for 21 days as 2.5 g ointment, the mean Cmax was 396.3±297.3 μg/mL.

By way of comparison, following an oral dose of 400 mg lopinavir (as Kaletra 400 mg/100 mg tablets) twice daily for 2 weeks, the mean Cmax for lopinavir was 12.3±5.4 μg/mL [SPMC Kaletra]. Adjusting for dose comparison with a 300 mg dose, the mean Cmax would be 9.23±4.1 μg/mL. The ratio of Cmax oral/Cmax topical is >23,000 indicating that less than 0.004% of the ointment comparative formulation 1 topical dose is available systemically.

### AC 0−t

For the ointment comparative formulation 1 (Example 4) containing 12% w/w lopinavir, following a topical dose of 300 mg lopinavir daily for 21 days as 2.5 g ointment, the AUC0−t was 7368.1±4973.1 μg/mL.

By way of comparison, following an oral dose of 400 mg lopinavir (as Kaletra 400 mg/100 mg tablets) twice daily for 2 weeks, the AUC0−t for lopinavir was 113.2±60.5 μg-h/mL [SPMC Kaletra]. Adjusting for dose comparison with a 300 mg dose, the AUC0−t would be 84.9±45.4 μg·h/mL. The ratio of AUC oral/AUC topical is >11,500 indicating that less than 0.009% of the ointment comparative formulation 1 topical dose is available systemically.

### Example 10: A Phase 1b, Multicentre, Open Label, Study of the Efficacy, Safety and Tolerability of Formulation 3p in Women with Cytological Abnormalities of the Uterine Cervix

The composition according to Example 3 (Formulation 3p) is investigated according to the clinical trial described below.

### Study Objectives

### Efficacy Objectives

Demonstrate histological clearance of cytological abnormalities following administration of Formulation 3p in women with high-grade or low-grade CIN (Cervical intra-epithelial neoplasia).
Demonstrate changes to colposcopic appearance of the uterine cervix following administration of Formulation 3p;
Assess changes in HPV status following administration of Formulation 3p.

### Safety Objective

Assess the incidence of AEs following the administration of Formulation 3p.

### Tolerability Objective

Assess the tolerability of Formulation 3p, measured by compliance with dosing schedule of Formulation 3p during 21 consecutive days of treatment.

### Primary Efficacy Endpoints:

Change in histology from screening to PTAV.

### Secondary Efficacy Endpoints:

Change in cytology from screening to PTAV as seen in cervical smear results
Changes in colposcopic appearance of disease from screening to PTAV.
Changes in HPV status (presence/absence of HPV genotypes) between screening and PTAV.

### Safety Endpoints:

Incidence of adverse events.
Changes from baseline in:
   Vital signs (blood pressure, heart rate, and temperature).
Safety clinical laboratory assessments (haematology, biochemistry, urinalysis).

### Tolerability Endpoint:

Review of participant diary cards to monitor application of the investigational product as per the protocol.

### Study Design:

This study is designed as a Phase 1b multicentre, open label study investigating the efficacy, safety and tolerability of Formulation 3p in women with cytological abnormalities of the uterine cervix who have been diagnosed with HPV.

In this single arm study, participants are stratified according to their grade of cytological abnormality:
Biopsy proven high-grade cytological abnormalities of the uterine cervix defined as CIN 2 and above;
Low-grade cytological abnormalities of the uterine cervix defined as CIN 1/LSIL.

A capsule filled with 1500 mg of formulation 3p—or alternatively two capsules each filled with 750 mg of formulation 3p—(therefore containing 300 mg lopinavir and 25 mg ritonavir) is self-inserted to the vagina once a day for 21 consecutive days. Participants complete a daily diary card and Vaginal Irritation Questionnaire (VIQ) to capture compliance with investigational product administration, AEs and changes to concomitant medication.

Each enrolled participant will complete 4 visits during the study—screening, treatment, end of treatment and follow-up visit as follows:
Visit 1—Screening Visit: Day −42 to Day 0.
Visit 2—Treatment:
Day 1® to Day 21: Treatment Cycle 1—investigational product adminstered once daily for 21 days;
Day 8, 15, 22: telephone follow up; check AEs, conmeds, compliance, dosing issues;
Visit 3—End of Treatment:
Day 28: visual assessment of disease #.
Visit 4—Follow-up (Post Treatment Assessment Visit—PTAV):
Between 3-6 weeks after end of treatment (last dose of Formulation 3p)—day 49-70.
   *Day 1 commences at the end of a participant's menstrual cycle.
   #If absence of disease, defined as no colposcopic evidence of CIN, participants are considered responders. If disease is detected, defined as ongoing colposcopic evidence of CIN, participants are considered non-responders.

### Participation Criteria

### Inclusion Criteria:

To be eligible for study entry participants must satisfy all of the following criteria:
1. Provision of written informed consent prior to any study specific procedures;
2. Female participants aged 22-50 years inclusive at the time of screening visit;
3. Positive result for cervical high-risk HPV (types 16, 18 or ‘other’);
4. High-grade cytological abnormality of the uterine cervix defined as CIN 2 and above as proven by colposcopic biopsy collected at screening, or within 30 days prior to screening;
OR
   low-grade cytological abnormality of the uterine cervix defined as CIN 1/LSIL, as demonstrated by colposcopic biopsy within 6 months prior to screening, or by biopsy collected at screening.
Participants will be stratified according to their grade of cytological abnormality;
5. Transformation zone needs to be fully visible;
6. Generally, in good health with no clinically significant disease as determined by the investigator;
7. Regular menstrual cycle with an approximate 28-day cycle
OR
   women who are amenorrhoeic due to effective contraception (such as Mirena, Jadelle, or continuous COC)
8. Agree to abstain from activities such as vaginal douching or insertion of any vaginal products other than the study drug for at least 48 hours prior to enrolment and throughout the study. Tampons may be used during the menstrual cycle only.
9. Women of childbearing potential (WOCBP) must use a highly effective form of birth control (confirmed by the Investigator). Rhythm methods will not be considered as highly effective methods of birth control. Highly effective forms of birth control include:
   True sexual abstinence (defined as refraining from heterosexual intercourse for the duration of the study and a minimum of 30 days following the last dose of study drug);
   Vasectomised partner (provided that the partner is the sole sexual partner of the female participant with childbearing potential and that the vasectomised partner has received medical assessment of the surgical success);
   Oral or transdermal combined (oestrogen and progestogen containing) hormonal contraception associated with inhibition of ovulation;
   Oral, injectable or implantable progestogen-only hormone contraception associated with inhibition of ovulation (Depo-Provera™, Implanon, or Cerazette, Noriday (‘mini-pill’));
   Any effective intrauterine device/levonorgestrel intrauterine system;
   Female sterilisation by tubal occlusion;
   Evra Patch™.

WOCBP must agree to use a highly effective method of birth control, as defined above, from enrolment, and at least 14 days prior to Day 1, throughout the study duration and within 30 days after the last dose of IMP.

WOCBP are defined as women who are neither permanently sterilised (hysterectomy, bilateral oophorectomy, or bilateral salpingectomy), nor who are postmenopausal. Women will be considered post-menopausal if they have been amenorrhoeic for 12 months or more without an alternative biological or medical cause e.g. contraceptive method such as Mirena.
10. Male partners of female participants must agree to use condoms during sexual intercourse from the first dose of investigational product until 30 days after the participants last dose to avoid potential transfer of investigational product.
11. Able and willing to abstain from sexual intercourse from 6 hours prior to dosing until 6 hours after dosing;
12. Ability and willingness to attend the necessary visits to the study centre;
13. Ability to comprehend all study related documentation, including written informed consent form, and complete all study-related tasks including daily diary;
14. Be willing and able to adhere to the prohibitions and restrictions specified in the protocol.

### Exclusion Criteria:

Participants are excluded from the study if one or more of the following criteria are applicable:
1. Any significant disease or disorder (e.g. cardiovascular, pulmonary, gastrointestinal, hepatic, renal, neurological, musculoskeletal, endocrine, metabolic, malignant, psychiatric, major physical impairment) which, in the opinion of the investigator, may either put the participant at risk because of participation in the study, or may influence the results of the study, or the participant's ability to participate in the study;
2. Any clinically significant abnormal findings in physical examination, vital signs, haematology, clinical chemistry, or urinalysis during screening and at baseline, which in the opinion of the investigator, may put the participant at risk because of her participation in the study, or may influence the results of the study, or the participant's ability to complete entire duration of the study;
3. Pregnant, breastfeeding, or lactating women (WOCBP must have a negative serum pregnancy test at screening and a negative urine pregnancy test at the start of treatment [i.e. Day 1]);
4. Women who plan to become pregnant in the next 6 months;
5. History of genital herpes with >3 outbreaks per year, or active non-HPV vaginal infection;
6. Active pelvic infection (positive for gonorrhoea or chlamydial infection, positive test for bacterial vaginosis, candida vaginitis or trichomonal vaginitis). Participants with positive results can be re-tested once during screening;
7. Positive bimanual exam consistent with pelvic inflammatory disease. Patients may be treated accordingly and re-screened;
8. Positive result for hepatitis B, hepatitis C or human immunodeficiency virus;
9. Current or recent abnormal vaginal discharge and/or abnormal vaginal bleeding, within the 3 months prior to Day 1 as assessed by the investigator;
10. Had an abortion or miscarriage or taken the morning-after pill within the 3 months prior to enrolment;
11. Currently taking immunosuppressants, intra-vaginal preparations, or any prescription that in the opinion of the investigator could be a potential safety issue or interfere with the interpretation of the results;
12. Previous exposure to lopinavir/ritonavir (within 3 months prior to screening), contraindication to the use of lopinavir/ritonavir or known allergy, hypersensitivity, or intolerance to any component of lopinavir/ritonavir formulation excipients;
13. Recent history (within 3 months prior to screening) of Stevens-Johnson syndrome, erythema multiforme, urticaria, angioedema, deep vein thrombosis, tinnitus, vertigo, blood glucose disorders, pancreatitis, haemophilia;
14. Receipt of any investigational product within 30 days or 5 half-lives prior to dosing;
15. Employees of the clinical study team or family members (first-degree relatives) of such individuals or anyone involved in the planning and/or conduct of the study. Clinical study team refers to employees directly involved in the study who have been delegated study-related tasks accordingly;
16. Participants who, in the opinion of the Investigator, do not understand the information and procedures of the study, or would not be compliant with them (in particular the study restrictions and risks involved).

### Dosing Schedule:

Investigational product will be self-administered by participants at approximately 10 pm (±1 hour) each evening. Participants will be provided dosing instructions and important application instructions.

Participants will record details of investigational product application in a diary card each day to monitor compliance. Participants are also asked to note in their diary card if there is any waste/spillage.

Participants will be required to bring their investigational product and diary card to the Day 28 clinic visit. The tube will be weighed prior to dispensing to the participant and again at the Day 28 visit to assess compliance.

There are no fasting requirements associated with the timing of application of investigational product.

### Safety Assessments

The following safety assessments are performed at timepoints outlined in the Schedule of Events, see Table 26.
Medical History
Physical Examination
Vital Signs
Body Weight and Height
12-lead Electrocardiogram
Clinical Laboratory Safety Tests (Haematology, Biochemistry, Urinalysis, Vaginal Microbiology, Viral Serology, Drugs of Abuse Screen, Alcohol Screen, Pregnancy Screen)

### Efficacy Assessments

The efficacy of the investigational product are assessed by improvements in the cytological abnormalities of the uterine cervix. Efficacy assessments are performed at timepoints outlined in the Schedule of Events, see Table 26.
Colposcopic visual assessment
Colposcopic Biopsy
Cytological Sampling
HPV Genotyping

TABLE 26

| | | | | | | | Post-treatment |
|---|---|---|---|---|---|---|---|
| | | | Treatment (21 | | | | assessment/Early |
| | Screening | consecutive days from Day 1) | | | | | Termination Visit |
| | Day | Day | Day | Day | Day | Day | 3-6 weeks after |
| Day | −42 to 0 | 1 | 8 | 15 | 22 | 28 | last dose of IMP |
| Attend study site | X | X | | | | X | X |
| Telephone Follow up | | | X | X | X | | |
| Informed consent | X | | | | | | |
| Inclusion/exclusion criteria | X | X | | | | | |
| Demographics | X | | | | | | |
| Medication history | X | | | | | | |
| Physical examination | X | | | | | | X |
| Vital signs | X | X | | | | X | X |
| Weight, height | X | | | | | | |
| 12-lead Safety ECG | X | | | | | | |
| Alcohol breath test | X | | | | | | |
| Drugs of abuse screen | X | X | | | | | |
| Concomitant medications | X | X | X | X | X | X | X |
| Adverse events | X | X | X | X | X | X | X |
| Haematology | X | | | | | X | X |
| Biochemistry | X | | | | | X | X |
| Serology | X | | | | | | |
| Urinalysis | X | | | | | X | X |
| Pregnancy Test | X | X | | | | X | X |
| Vaginal microbiology | X | | | | | X | X |
| Vaginal pH | X | | | | | X | X |
| Papanicolaou smear | X | | | | | | X |
| HPV genotyping | X | | | | | | X |
| Colposcopy visual | X | | | | | X | X |
| assessment | | | | | | | |
| Colposcopic biopsy | X | | | | | | X |
| Investigational product | | X | | | | | |
| dispensed | | | | | | | |
| Participant diary/VIQ | | X— | — | — | — | X | |
| completion | | | | | | | |
| Review diary & VIQ | | | | | | X | |

1. A self-emulsifying pharmaceutical composition comprising:
a. an unsaturated free fatty acid;
b. at least two emulsifiers; and
c. lopinavir;
wherein the at least two emulsifiers comprise at least a first emulsifier which has a HLB value greater than about 14 and at least a second emulsifier which has a HLB value less than about 6; and wherein the total emulsifier content is less than 30% by weight of the total composition.

2. The pharmaceutical composition according to claim 1, wherein the unsaturated free fatty acid is oleic acid.

3. The pharmaceutical composition according to claim 1, wherein the first emulsifier has a HLB value selected from the group consisting of greater than about 15, greater than about 16, greater than about 17, and greater than about 18.

4. The pharmaceutical composition according to claim 1, wherein the first emulsifier is a polyol ester selected from the group consisting of a polyol stearate (PEG100 stearate, a polyethoxylated sorbitan ester, and polysorbate 20.

5. The pharmaceutical composition according to claim 1, wherein the second emulsifier has a HLB value less than about 5.5, less than about 5, less than about 4.5, or less than about 4.

6. The pharmaceutical composition according to claim 1, wherein the second emulsifier is a monoglyceride.

7. The pharmaceutical composition according to claim 1, wherein the wt/wt ratio of second emulsifier to first emulsifier present in the composition is between about 1:10 and about 10:1, between about 1:5 and about 5:1, between about 1:3 and about 3:1, between about 1:1 and about 5:1, between about 1:1 and about 3:1, between about 1:1 and about 2:1, between about 1:2 and about 2:1, between about 1:1.5 and about 2:1, between about 1:1.3 and about 1:1.1, between about 1:1.2, between about 1.1:1 and 1.2:1, between about 1.2:1, between about 1.4:1 and 1.6:1, or between about 1.5:1.

8. The pharmaceutical composition according to claim 1, wherein the pharmaceutical composition further comprises at least a third emulsifier.

9. The pharmaceutical composition according to claim 8, wherein the first emulsifier has a HLB value greater than 14, the second emulsifier has a HLB value less than 6, the third emulsifier has a HLB value in the range 8 to 15; and the total emulsifier content is less than 30% by weight of the total composition.

10. The pharmaceutical composition according to claim 8, wherein the third emulsifier is a polyoxyl castor oil derivative.

11. The pharmaceutical composition according to claim 1, wherein the total emulsifier content is less than 25% by weight, less than 20% by weight, less than 15% by weight, 2% to 20% by weight, 2.5% to 15% by weight, 5% to 15% by weight, 8% to 12%, 10% to 20%, 12% to 20%, 10% to 18%, 12% to 18%, 12% to 16%, 13% to 20%, 14% to 20%, 15% to 20%, 13% to 25%, 14% to 25%, 15% to 25%, 20% to 25%, 20% to 26%, 20% to 27%, 20% to 28% or 20% to 29% by weight of the total composition.

12. The pharmaceutical composition according to claim 1, wherein the first emulsifier is present in the pharmaceutical composition at about 1% to about 20% by weight of the total pharmaceutical composition, about 1% to about 10% by weight, about 10% to about 20% by weight, about 10% to about 15% by weight, about 1% to about 5% by weight, about 3% to about 7% by weight, about 3% to about 6% by weight, about 3% to about 5% by weight, about 4% by weight, about 4% to about 5% by weight, about 4.3% by weight, or about 5% to about 6% by weight, or about 5.5% by weight of the total composition.

13. The pharmaceutical composition according to claim 1, wherein the second emulsifier is present in the pharmaceutical composition at about 1% to about 20% by weight of the total pharmaceutical composition, about 1% to about 10% by weight, about 2% to about 8% by weight, about 4% to about 7% by weight, about 5% to about 7% by weight, about 6% by weight, about 4% to about 6% by weight, about 5% by weight, or about 4% to about 5% by weight, or about 4.5% by weight of the total composition.

14. The pharmaceutical composition according to claim 8, wherein the third emulsifier is present in the pharmaceutical composition at about 1% to about 10% by weight of the total pharmaceutical composition, about 2% to about 8% by weight, about 3% to about 7% by weight, about 4% to about 6% by weight, or about 5% by weight of the total composition

15. The pharmaceutical composition according to claim 1, wherein the pharmaceutical composition further comprises ritonavir.

16. The pharmaceutical composition according to claim 15, wherein the wt/wt ratio of lopinavir to ritonavir present in the composition is between about 1:10 and about 18:1, between about 1:10 and about 15:1, between about 1:5 and about 15:1, between about 1:1 and about 15:1, between about 2:1 and about 15:1, between about 4:1 and about 15:1, between about 8:1 and about 14:1, between about 9:1 and about 14:1, between about 10:1 and about 14:1, between 10.5:1 and about 18:1, between 10.5:1 and 18:1, between about 10.5:1 and about 14:1, between about 11:1 to about 13:1, between about 11.5 and about 17:1, between about 11.5:1 and about 16.0:1, between about 11.5:1 and about 15:1, about 14.5:1, 14.5:1, about 14:1, 14:1, about 13.8:1, 13.8:1, about 13.75:1, 13.75:1, about 13.5:1, 13.5:1, about 13:1, 13:1, about 12.5:1, 12.5:1, about 12:1, 12:1, about 11.75:1, 11.75:1, about 11.5:1, 11.5:1, about 11.25:1, 11.25:1, about 11:1, or 11:1.

17. A self-emulsifying pharmaceutical composition consisting of:
a. an unsaturated free fatty acid;
b. a first emulsifier having a HLB value greater than about 14;
c. a second emulsifier having a HLB value less than about 6;
d. a third emulsifier;
e. an antioxidant;
f. lopinavir; and
g. ritonavir;
wherein the total emulsifier content is less than 30% by weight of the total composition.

18. The pharmaceutical composition according to claim 1 wherein the pharmaceutical composition is encapsulated within a capsule.

19. The pharmaceutical composition according to claim 1 for use as a medicament.

20. A method of treating and/or inhibiting the development or progression of cancers and benign proliferative disorders in a subject in need of such treatment or inhibition comprising administering a therapeutically effective amount of a pharmaceutical composition according to claim 1 to said subject.

21. A method of treating a patient having an HPV related dysplasia of the cervix comprising administering intra-vaginally or orally to said patient a therapeutically effective dose of a pharmaceutical composition according to claim 1.

22. The method of claim 21, wherein the pharmaceutical composition reduces the severity of the HPV related dysplasia.

23. The method of claim 21, wherein the composition induces apoptosis of HPV infected cells.

24. The method of claim 21, wherein the patient has a cervical cytology of high grade squamous intraepithelial lesion (HSIL), atypical squamous cells of undetermined significance (ASCUS), or low grade squamous intraepithelial lesion (LSIL).

25. A process to manufacture a self-emulsifying pharmaceutical composition of claim 1, the process comprising the steps of:
a. incorporating lopinavir in an unsaturated free fatty acid;
b. incorporating at least two emulsifiers to the mixture from step a) to provide a self-emulsifying composition;
wherein the at least two emulsifiers comprise a first emulsifier having a HLB value greater than about 14 and a second emulsifier having a HLB value less than about 6; and wherein the total emulsifier content is less than 30% by weight of the total composition.

26. The method of claim 20, wherein the pharmaceutical composition reduces the severity of the HPV related dysplasia.

27. The pharmaceutical composition according to claim 1 wherein the second emulsifier is a glycerol monooleate.

28. The pharmaceutical composition according to claim 8 wherein the third emulsifier is PEG35 castor oil.
